(19) United States
(12) Patent Application Publication
(10) Pub. No.: US 2009/0123912 A1
(43) Pub. Date: May 14, 2009

(54) Methods for quantitating small RNA molecules

(75) Inventor: Christopher K. Raymond, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Rosetta Inpharmatics LLC, Seattle, WA (US)

(21) Appl. No.: 10/579,029

(22) PCT Filed: Jan. 25, 2006
(86) PCT No.: PCT/US06/02591

§ 371 Date: Nov. 19, 2008

Related U.S. Application Data
(60) Provisional application No. 60/647,178, filed on Jan. 25, 2005.

(51) Int. Cl. C12Q 1/68; C12P 19/34; C07H 21/04

(52) U.S. Cl. 435/6; 435/91.2; 536/24.33

(57) ABSTRACT

In one aspect, the present invention provides methods for amplifying a microRNA molecule to produce DNA molecules. The methods each include the steps of: (a) using primer extension to make a DNA molecule that is complementary to a target microRNA molecule; and (b) using a universal forward primer and a reverse primer to amplify the DNA molecule to produce amplified DNA molecules. In some embodiments of the method, at least one of the forward primer and the reverse primer comprise at least one locked nucleic acid molecule.

### FIELD OF THE INVENTION

The present invention relates to methods of amplifying and quantitating small RNA molecules.

### BACKGROUND OF THE INVENTION

RNA interference (RNAi) is an evolutionarily conserved process that functions to inhibit gene expression (Bernstein et al. (2001), Nature 409:363-6; Dykxhoorn et al. (2003) Nat. Rev. Mol. Cell. Biol. 4:457-67). The phenomenon of RNAi was first described in Caenorhabditis elegans, where injection of double-stranded RNA (dsRNA) led to efficient sequence-specific gene silencing of the mRNA that was complementary to the dsRNA (Fire et al. (1998) Nature 391:806-11). RNAi has also been described in plants as a phenomenon called post-transcriptional gene silencing (PTGS), which is likely used as a viral defense mechanism (Jorgensen (1990) Trends Biotechnol. 8:340-4; Brigneti et al. (1998) EMBO J. 17:6739-46; Hamilton & Baulcombe (1999) Science 286:950-2).

An early indication that the molecules that regulate PTGS were short RNAs processed from longer dsRNA was the identification of short 21 to 22 nucleotide dsRNA derived from the longer dsRNA in plants (Hamilton & Baulcombe (1999) Science 286:950-2). This observation was repeated in Drosophila embryo extracts where long dsRNA was found processed into 21-25 nucleotide short RNA by the RNase III type enzyme, Dicer (Elbashir et al. (2001) Nature 411:494-8; Elbashir et al. (2001) EMBO J. 20:6877-88; Elbashir et al. (2001) Genes Dev. 15:188-200). These observations led Elbashir et al. to test if synthetic 21-25 nucleotide synthetic dsRNAs function to specifically inhibit gene expression in Drosophila embryo lysates and mammalian cell culture (Elbashir et al. (2001) Nature 411:494-8; Elbashir et al. (2001) EMBO J. 20:6877-88; Elbashir et al. (2001) Genes Dev. 15:188-200). They demonstrated that small interfering RNAs (siRNAs) had the ability to specifically inhibit gene expression in mammalian cell culture without induction of the interferon response.

These observations led to the development of techniques for the reduction, or elimination, of expression of specific genes in mammalian cell culture, such as plasmid-based systems that generate hairpin siRNAs (Brummelkamp et al. (2002) Science 296:550-3; Paddison et al. (2002) Genes Dev. 16:948-58; Paddison et al. (2002) Proc. Natl. Acad. Sci. U.S.A. 99:1443-8; Paul et al. 2002) Nat. Biotechnol. 20:404-8). siRNA molecules can also be introduced into cells, in vivo, to inhibit the expression of specific proteins (see, e.g., Soutschek, J., et al., Nature 432 (7014):173-178 (2004)).

siRNA molecules have promise both as therapeutic agents for inhibiting the expression of specific proteins, and as targets for drugs that affect the activity of siRNA molecules that function to regulate the expression of proteins involved in a disease state. A first step in developing such therapeutic agents is to measure the amounts of specific siRNA molecules in different cell types within an organism, and thereby construct an “atlas” of siRNA expression within the body. Additionally, it will be useful to measure changes in the amount of specific siRNA molecules in specific cell types in response to a defined stimulus, or in a disease state.

Short RNA molecules are difficult to quantitate. For example, with respect to the use of PCR to amplify and measure the small RNA molecules, most PCR primers are longer than the small RNA molecules, and so it is difficult to design a primer that has significant overlap with a small RNA molecule, and that selectively hybridizes to the small RNA molecule at the temperatures used for primer extension and PCR amplification reactions.

### SUMMARY OF THE INVENTION

In one aspect, the present invention provides methods for amplifying a microRNA molecule to produce cDNA molecules. The methods include the steps of: (a) producing a first DNA molecule that is complementary to a target microRNA molecule using primer extension; and (b) amplifying the first DNA molecule to produce amplified DNA molecules using a universal forward primer and a reverse primer. In some embodiments of the method, at least one of the forward primer and the reverse primer comprise at least one locked nucleic acid molecule. It will be understood that, in the practice of the present invention, typically numerous (e.g., millions) of individual microRNA molecules are amplified in a sample (e.g., a solution of RNA molecules isolated from living cells).

In another aspect, the present invention provides methods for measuring the amount of a target microRNA in a a sample from a living organism. The methods of this aspect of the invention include the step of measuring the amount of a target microRNA molecule in a multiplicity of different cell types within a living organism, wherein the amount of the target microRNA molecule is measured by a method including the steps of: (1) producing a first DNA molecule complementary to the target microRNA molecule in the sample using primer extension; (2) amplifying the first DNA molecule to produce amplified DNA molecules using a universal forward primer and a reverse primer; and (3) measuring the amount of the amplified DNA molecules. In some embodiments of the method, at least one of the forward primer and the reverse primer comprise at least one locked nucleic acid molecule.

In another aspect, the invention provides nucleic acid primer molecules consisting of sequence SEQ ID NO:1 to SEQ ID NO: 499, as shown in TABLE 1, TABLE 2, TABLE 6 and TABLE 7. The primer molecules of the invention can be used as primers for detecting mammalian microRNA target molecules, using the methods of the invention described herein.

In another aspect, the present invention provides kits for detecting at least one mammalian target microRNA, the kits comprising one or more primer sets specific for the detection of a target microRNA, each primer set comprising (1) an extension primer for producing a cDNA molecule complementary to a target microRNA, (2) a universal forward PCR primer for amplifying the cDNA molecule and (3) a reverse PCR primer for amplifying the cDNA molecule. The extension primer comprises a first portion that hybridizes to the target microRNA molecule and a second portion that includes a hybridization sequence for a universal forward PCR primer. The reverse PCR primer comprises a sequence selected to hybridize to a portion of the cDNA molecule. In some embodiments of the kit, at least one of the universal forward and reverse primers include at least one locked nucleic acid molecule. The kits of the invention may be used to practice various embodiments of the methods of the invention.

The present invention is useful, for example, for quantitating specific microRNA molecules within different types of cells in a living organism, or, for example, for measuring changes in the amount of specific microRNAs in living cells in response to a stimulus (e.g., in response to administration of a drug).

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a flow chart of a representative method of the present invention;

FIG. 2 graphically illustrates the standard curves for assays specific for the detection of microRNA targets miR-95 and miR-424 as described in EXAMPLE 3;

FIG. 3A is a histogram plot showing the expression profile of miR-1 across a panel of total RNA isolated from twelve tissues as described in EXAMPLE 5;

FIG. 3B is a histogram plot showing the expression profile of miR-124 across a panel of total RNA isolated from twelve tissues as described in EXAMPLE 5; and

FIG. 3C is a histogram plot showing the expression profile of miR-150 across a panel of total RNA isolated from twelve tissues as described in EXAMPLE 5.

### DETAILED DESCRIPTION OF THE PREFERRED EMBODIMENT

In accordance with the foregoing, in one aspect, the present invention provides methods for amplifying a microRNA molecule to produce cDNA molecules. The methods include the steps of: (a) using primer extension to make a DNA molecule that is complementary to a target microRNA molecule; and (b) using a universal forward primer and a reverse primer to amplify the DNA molecule to produce amplified DNA molecules. In some embodiments of the method, at least one of the universal forward primer and the reverse primer comprises at least one locked nucleic acid molecule.

As used, herein, the term “locked nucleic acid molecule” (abbreviated as LNA molecule) refers to a nucleic acid molecule that includes a 2′-O,4′-C-methylene-β-D-ribofuranosyl moiety. Exemplary 2′-O,4′-C-methylene-β-D-ribofuranosyl moieties, and exemplary LNAs including such moieties, are described, for example, in Petersen, M. and Wengel, J., Trends in Biotechnology 21(2):74-81 (2003) which publication is incorporated herein by reference in its entirety.

As used herein, the term “microRNA” refers to an RNA molecule that has a length in the range of from 21 nucleotides to 25 nucleotides. Some microRNA molecules (e.g., siRNA molecules) function in living cells to regulate gene expression.

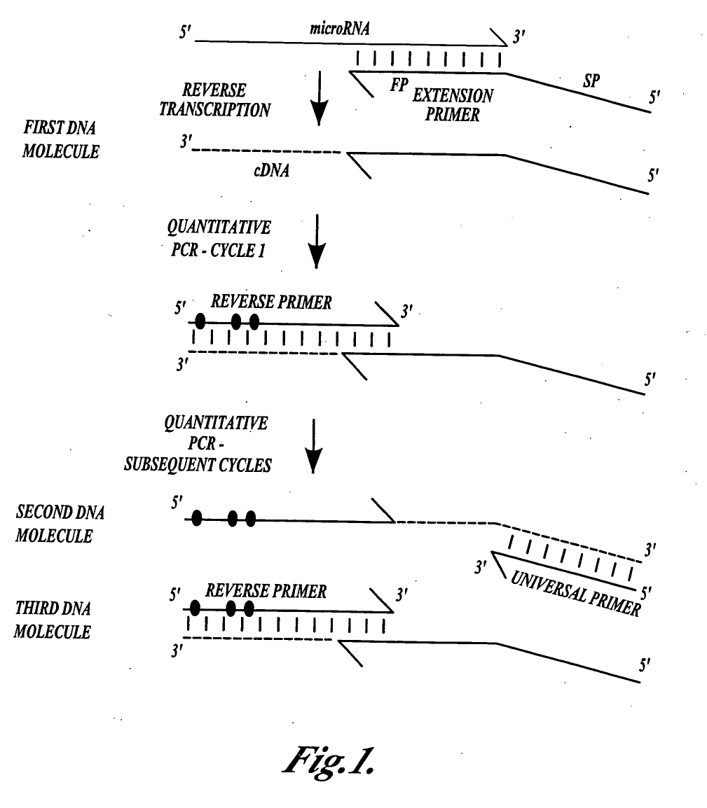

Representative method of the invention. FIG. 1 shows a flowchart of a representative method of the present invention. In the method represented in FIG. 1, a microRNA is the template for synthesis of a complementary first DNA molecule. The synthesis of the first DNA molecule is primed by an extension primer, and so the first DNA molecule includes the extension primer and newly synthesized DNA (represented by a dotted line in FIG. 1). The synthesis of DNA is catalyzed by reverse transcriptase.

The extension primer includes a first portion (abbreviated as FP in FIG. 1) and a second portion (abbreviated as SP in FIG. 1). The first portion hybridizes to the microRNA target template, and the second portion includes a nucleic acid sequence that hybridizes with a universal forward primer, as described infra.

A quantitative polymerase chain reaction is used to make a second DNA molecule that is complementary to the first DNA molecule. The synthesis of the second DNA molecule is primed by the reverse primer that has a sequence that is selected to specifically hybridize to a portion of the target first DNA molecule. Thus, the reverse primer does not hybridize to nucleic acid molecules other than the first DNA molecule. The reverse primer may optionally include at least one LNA molecule located within the portion of the reverse primer that does not overlap with the extension primer. In FIG. 1, the LNA molecules are represented by shaded ovals.

A universal forward primer hybridizes to the 3′ end of the second DNA molecule and primes synthesis of a third DNA molecule. It will be understood that, although a single microRNA molecule, single first DNA molecule, single second DNA molecule, single third DNA molecule and single extension, forward and reverse primers are shown in FIG. 1, typically the practice of the present invention uses reaction mixtures that include numerous copies (e.g., millions of copies) of each of the foregoing nucleic acid molecules.

The steps of the methods of the present invention are now considered in more detail.

Preparation of microRNA molecules useful as templates. microRNA molecules useful as templates in the methods of the invention can be isolated from any organism (e.g., eukaryote, such as a mammal) or part thereof, including organs, tissues, and/or individual cells (including cultured cells). Any suitable RNA preparation that includes microRNAs can be used, such as total cellular. RNA.

RNA may be isolated from cells by procedures that involve lysis of the cells and denaturation of the proteins contained therein. Cells of interest include wild-type cells, drug-exposed wild-type cells, modified cells, and drug-exposed modified cells.

Additional steps may be employed to remove some or all of the DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (see, Chirgwin et al., 1979, Biochemistry 18:5294-5299). Separation of RNA from DNA can also be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

The sample of RNA can comprise a multiplicity of different microRNA molecules, each different microRNA molecule having a different nucleotide sequence. In a specific embodiment, the microRNA molecules in the RNA sample comprise at least 100 different nucleotide sequences. In other embodiments, the microRNA molecules of the RNA sample comprise at least 500, 1,000, 5,000, 10,000, 20,000, 30,000, 40,000, 50,000, 60,000, 70,000, 80,000, 90,000, or 100,000 different nucleotide sequences.

The methods of the invention may be used to detect the presence of any microRNA. For example, the methods of the invention can be used to detect one or more of the microRNA targets described in a database such as “the miRBase sequence database” as described in Griffith-Jones et al. (2004), Nucleic Acids Research 32:D109-D111, and Griffith-Jones et al. (2006), Nucleic Acids Research 34: D140-D144, which is publicly accessible on the World Wide Web at the Wellcome Trust Sanger Institute website at http://microrna.sanger.ac.uk/sequences/. A list of exemplary microRNA targets is also described in the following references: Lagos-Quintana et al., Curr. Biol. 12(9):735-9 (2002).

Synthesis of DNA molecules using microRNA molecules as templates. In the practice of the methods of the invention, first DNA molecules are synthesized that are complementary to the microRNA target molecules, and that are composed of an extension primer and newly synthesized DNA (wherein the extension primer primes the synthesis of the newly synthesized DNA). Individual first DNA molecules can be complementary to a whole microRNA target molecule, or to a portion thereof; although typically an individual first DNA molecule is complementary to a whole microRNA target molecule. Thus, in the practice of the methods of the invention, a population of first DNA molecules is synthesized that includes individual DNA molecules that are each complementary to all, or to a portion, of a target microRNA molecule.

The synthesis of the first DNA molecules is catalyzed by reverse transcriptase. Any reverse transcriptase molecule can be used to synthesize the first DNA molecules, such as those derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT). A reverse transcriptase lacking RNaseH activity (e.g., SUPERSCRIPT III™ sold by Invitrogen, 1600 Faraday Avenue, PO Box 6482, Carlsbad, Calif. 92008) is preferred in order to minimize the amount of double-stranded cDNA synthesized at this stage. The reverse transcriptase molecule should also preferably be thermostable so that the DNA synthesis reaction can be conducted at as high a temperature as possible, while still permitting hybridization of primer to the microRNA target molecules.

Priming the synthesis of the first DNA molecules. The synthesis of the first DNA molecules is primed using an extension primer. Typically, the length of the extension primer is in the range of from 10 nucleotides to 100 nucleotides, such as 20 to 35 nucleotides. The nucleic acid sequence of the extension primer is incorporated into the sequence of each, synthesized, DNA molecule. The extension primer includes a first portion that hybridizes to a portion of the microRNA molecule. Typically the first portion of the extension primer includes the 3′-end of the extension primer. The first portion of the extension primer typically has a length in the range of from 6 nucleotides to 20 nucleotides, such as from 10 nucleotides to 12 nucleotides. In some embodiments, the first portion of the extension primer has a length in the range of from 3 nucleotides to 25 nucleotides.

The extension primer also includes a second portion that typically has a length of from 18 to 25 nucleotides. For example, the second portion of the extension primer can be 20 nucleotides long. The second portion of the extension primer is located 5′ to the first portion of the extension primer. The second portion of the extension primer includes at least a portion of the hybridization site for the universal forward primer. For example, the second portion of the extension primer can include all of the hybridization site for the universal forward primer, or, for example, can include as little as a single nucleotide of the hybridization site for the universal forward primer (the remaining portion of the hybridization site for the forward primer can, for example, be located in the first portion of the extension primer). An exemplary nucleic acid sequence of a second portion of an extension primer is 5′ CATGATCAGCTGGGCCAAGA 3′ (SEQ ID NO:1).

Amplification of the DNA molecules. In the practice of the methods of the invention, the first DNA molecules are enzymatically amplified using the polymerase chain reaction. A universal forward primer and a reverse primer are used to prime the polymerase chain reaction. The reverse primer includes a nucleic acid sequence that is selected to specifically hybridize to a portion of a first DNA molecule.

The reverse primer typically has a length in the range of from 10 nucleotides to 100 nucleotides. In some embodiments, the reverse primer has a length in the range of from 12 nucleotides to 20 nucleotides. The nucleotide sequence of the reverse primer is selected to hybridize to a specific target nucleotide sequence under defined hybridization conditions. The reverse primer and extension primer are both present in the PCR reaction mixture, and so the reverse primer should be sufficiently long so that the melting temperature (Tm) is at least 50° C., but should not be so long that there is extensive overlap with the extension primer which may cause the formation of “primer dimers.” “Primer dimers” are formed when the reverse primer hybridizes to the extension primer, and uses the extension primer as a substrate for DNA synthesis, and the extension primer hybridizes to the reverse primer, and uses the reverse primer as a substrate for DNA synthesis. To avoid the formation of “primer dimers,” typically the reverse primer and the extension primer are designed so that they do not overlap with each other by more than 6 nucleotides. If it is not possible to make a reverse primer having a Tm of at least 50° C., and wherein the reverse primer and the extension primer do not overlap by more than 6 nucleotides, then it is preferable to lengthen the reverse primer (since Tm usually increases with increasing oligonucleotide length) and decrease the length of the extension primer.

The reverse primer primes the synthesis of a second DNA molecule that is complementary to the first DNA molecule. The universal forward primer hybridizes to the portion of the second DNA molecule that is complementary to the second portion of the extension primer which is incorporated into all of the first DNA molecules. The universal forward primer primes the synthesis of third DNA molecules. The universal forward primer typically has a length in the range of from 16 nucleotides to 100 nucleotides. In some embodiments, the universal forward primer has a length in the range of from 16 nucleotides to 30 nucleotides. The universal forward primer may include at least one locked nucleic acid molecule. In some embodiments, the universal forward primer includes from 1 to 25 locked nucleic acid molecules. The nucleic acid sequence of an exemplary universal forward primer is set forth in SEQ ID NO:13.

In general, the greater the number of amplification cycles during the polymerase chain reaction, the greater the amount of amplified DNA that is obtained. On the other hand, too many amplification cycles (e.g., more than 35 amplification cycles) may result in spurious and unintended amplification of non-target double-stranded DNA. Thus, in some embodiments, a desirable number of amplification cycles is between one and 45 amplification cycles, such as from one to 25 amplification cycles, or such as from five to 15 amplification cycles, or such as ten amplification cycles.

Use of LNA molecules and selection of primer hybridization conditions: hybridization conditions are selected that promote the specific hybridization of a primer molecule to the complementary sequence on a substrate molecule. With respect to the hybridization of a 12 nucleotide first portion of an extension primer to a microRNA, it has been found that specific hybridization occurs at a temperature of 50° C. Similarly, it has been found that hybridization of a 20 nucleotide universal forward primer to a complementary DNA molecule, and hybridization of a reverse primer (having a length in the range of from 12-20 nucleotides, such as from 14-16 nucleotides) to a complementary DNA molecule occurs at a temperature of 50° C. By way of example, it is often desirable to design extension, reverse and universal forward primers that each have a hybridization temperature in the range of from 50° C. to 60° C.

In some embodiments, LNA molecules can be incorporated into at least one of the extension primer, reverse primer, and universal forward primer to raise the Tm of one, or more, of the foregoing primers to at least 5° C. Incorporation of an LNA molecule into the portion of the reverse primer that hybridizes to the target first DNA molecule, but not to the extension primer, may be useful because this portion of the reverse primer is typically no more than 10 nucleotides in length. For example, the portion of the reverse primer that hybridizes to the target first DNA molecule, but not to the extension primer, may include at least one locked nucleic acid molecule (e.g., from 1 to 25 locked nucleic acid molecules). In some embodiments, two or three locked nucleic acid molecules are included within the first 8 nucleotides from the 5′ end of the reverse primer.

The number of LNA residues that must be incorporated into a specific primer to raise the Tm to a desired temperature mainly depends on the length of the primer and the nucleotide composition of the primer. A tool for determining the effect on Tm of one or more LNAs in a primer is available on the Internet Web site of Exiqon, Bygstubben 9, DK-2950 Vedbaek, Denmark.

Although one or more LNAs can be included in any of the primers used in the practice of the present invention, it has been found that the efficiency of synthesis of cDNA is low if an LNA is incorporated into the extension primer. While not wishing to be bound by theory, LNAs may inhibit the activity of reverse transcriptase.

Detecting and measuring the amount of the amplified DNA molecules: the amplified DNA molecules can be detected and quantitated by the presence of detectable marker molecules, such as fluorescent molecules. For example, the amplified DNA molecules can be detected and quantitated by the presence of a dye (e.g., SYBR green) that preferentially or exclusively binds to double stranded DNA during the PCR amplification step of the methods of the present invention. For example, Molecular Probes, Inc. (29851 Willow Creek Road, Eugene, Oreg. 97402) sells quantitative PCR reaction mixtures that include SYBR green dye. By way of further example, another dye (referred to as “BEBO”) that can be used to label double stranded DNA produced during real-time PCR is described by Bengtsson, M., et al., Nucleic Acids Research 31(8):e45 (Apr. 15, 2003), which publication is incorporated herein by reference. Again by way of example, a forward and/or reverse primer that includes a fluorophore and quencher can be used to prime the PCR amplification step of the methods of the present invention. The physical separation of the fluorophore and quencher that occurs after extension of the labeled primer during PCR permits the fluorophore to fluoresce, and the fluorescence can be used to measure the amount of the PCR amplification products. Examples of commercially available primers that include a fluorophore and quencher include Scorpion primers and Uniprimers, which are both sold by Molecular Probes, Inc.

Representative uses of the present invention: The present invention is useful for producing cDNA molecules from microRNA target molecules. The amount of the DNA molecules can be measured which provides a measurement of the amount of target microRNA molecules in the starting material. For example, the methods of the present invention can be used to measure the amount of specific microRNA molecules (e.g., specific siRNA molecules) in living cells. Again by way of example, the present invention can be used to measure the amount of specific microRNA molecules (e.g., specific siRNA molecules) in different cell types in a living body, thereby producing an “atlas” of the distribution of specific microRNA molecules within the body. Again by way of example, the present invention can be used to measure changes in the amount of specific microRNA molecules (e.g., specific siRNA molecules) in response to a stimulus, such as in response to treatment of a population of living cells with a drug.

Thus, in another aspect, the present invention provides methods for measuring the amount of a target microRNA in a multiplicity of different cell types within a living organism (e.g., to make a microRNA “atlas” of the organism). The methods of this aspect of the invention each include the step of measuring the amount of a target microRNA molecule in a multiplicity of different cell types within a living organism, wherein the amount of the target microRNA molecule is measured by a method comprising the steps of: (1) using primer extension to make a DNA molecule complementary to the target microRNA molecule isolated from a cell type of a living organism; (2) using a universal forward primer and a reverse primer to amplify the DNA molecule to produce amplified DNA molecules, and (3) measuring the amount of the amplified DNA molecules. In some embodiments of the methods, at least one of the forward primer and the reverse primer comprises at least one locked nucleic acid molecule. The measured amounts of amplified DNA molecules can, for example, be stored in an interrogatable database in electronic form, such as on a computer-readable medium (e.g., a floppy disc).

In another aspect, the invention provides nucleic acid primer molecules consisting of sequence SEQ ID NO:1 to SEQ ID NO: 499, as shown in TABLE 1, TABLE 2, TABLE 6 and TABLE 7. The primer molecules of the invention can be used as primers for detecting mammalian microRNA target molecules, using the methods of the invention described herein.

In another aspect, the present invention provides kits for detecting at least one mammalian target microRNA, the kits comprising one or more primer sets specific for the detection of a target microRNA, each primer set comprising (1) an extension primer for producing a cDNA molecule complementary to a target microRNA, (2) a universal forward PCR primer and (3) a reverse PCR primer for amplifying the cDNA molecule. The extension primer comprises a first portion that hybridizes to the target microRNA molecule and a second portion that includes a hybridization sequence for a universal forward PCR primer. The reverse PCR primer comprises a sequence selected to hybridize to a portion of the cDNA molecule. In some embodiments of the kits, at least one of the universal forward and reverse primers includes at least one locked nucleic acid molecule.

The extension primer, universal forward and reverse primers for inclusion in the kit may be designed to detect any mammalian target microRNA in accordance with the methods described herein. Nonlimiting examples of human target microRNA target molecules and exemplary target-specific extension primers and reverse primers are listed below in TABLE 1, TABLE 2 and TABLE 6. Nonlimiting examples of murine target microRNA target molecules and exemplary target-specific extension primers and reverse primers are listed below in TABLE 7. A nonlimiting example of a universal forward primer is set forth as SEQ ID NO: 13.

In certain embodiments, the kit includes a set of primers comprising an extension primer, reverse and universal forward primers for a selected target microRNA molecule that each have a hybridization temperature in the range of from 50° C. to 60° C.

In certain embodiments, the kit includes a plurality of primer sets that may be used to detect a plurality of mammalian microRNA targets, such as two microRNA targets up to several hundred microRNA targets.

In certain embodiments, the kit comprises one or more primer sets capable of detecting at least one or more of the following human microRNA target templates: of miR-1, miR-7, miR-9*, miR-10a, miR-10b, miR-15a, miR-15b, miR-16, miR-17-3p, miR-17-5p, miR-18, miR-19a, miR-19b, miR-20, miR-21, miR-22, miR-23a, miR-23b, miR-24, miR-25, miR-26a, miR-26b, miR-27a, miR-28, miR-29a, miR-29b, miR-29c, miR-30a-5p, miR-30b, miR-30c, miR-30d, miR-30e-5p, miR-30e-3p, miR-31, miR-32, miR-33, miR-34a, miR-34b, miR-34c, miR-92, miR-93, miR-95, miR-96, miR-98, miR-99a, miR-99b, miR-100, miR-101, miR-103, miR-105, miR-106a, miR-107, miR-122, miR-122a, miR-124, miR-124, miR-124a, miR-125a, miR-125b, miR-126, miR-126*, miR-127, miR-128a, miR-128b, miR-129, miR-130a, miR-130b, miR-132, miR-133a, miR-133b, miR-134, miR-135a, miR-135b, miR-136, miR-137, miR-138, miR-139, miR-140, miR-141, miR-142-3p, miR-143, miR-144, miR-145, miR-146, miR-147, miR-148a, miR-148b, miR-149, miR-150, miR-151, miR-152, miR-153, miR-154*, miR-154, miR-155, miR-181a, miR-181b, miR-181c, miR-182*, miR-182, miR-183, miR-184, miR-185, miR-186, miR-187, miR-188, miR-189, miR-190, miR-191, miR-192, miR-193, miR-194, miR-195, miR-196a, miR-196b, miR-197, miR-198, miR-199a*, miR-199a, miR-199b, miR-200a, miR-200b, miR-200c, miR-202, miR-203, miR-204, miR-205, miR-206, miR-208, miR-210, miR-211, miR-212, miR-213, miR-213, miR-214, miR-215, miR-216, miR-217, miR-218, miR-220, miR-221, miR-222, miR-223, miR-224, miR-296, miR-299, miR-301, miR-302a*, miR-302a, miR-302b*, miR-302b, miR-302d, miR-302c*, miR-302c, miR-320, miR-323, miR-324-3p, miR-324-5p, miR-325, miR-326, miR-328, miR-330, miR-331, miR-337, miR-338, miR-339, miR-340, miR-342, miR-345, miR-346, miR-363, miR-367, miR-368, miR-370, miR-371, miR-372, miR-373*, miR-373, miR-374, miR-375, miR-376b, miR-378, miR-379, miR-380-5p, miR-380-3p, miR-381, miR-382, miR-383, miR-410, miR-412, miR-422a, miR-422b, miR-423, miR-424, miR-425, miR-429, miR-431, miR-448, miR-449, miR-450, miR-451, let7a, let7b, let7c, let7d, let7e, let7f, let7g, let7i, miR-376a, and miR-377. The sequences of the above-mentioned microRNA targets are provided in “the miRBase sequence database” as described in Griffith-Jones et al. (2004), Nucleic Acids Research 32:D109-D111, and Griffith-Jones et al. (2006), Nucleic Acids Research 34: D140-D144, which is publicly accessible on the World Wide Web at the Welcome Trust Sanger Institute website at http://microrna.sanger.ac.uk/sequences/.

Exemplary primers for use in accordance with this embodiment of the kit are provided in TABLE 1, TABLE 2 and TABLE 6 below.

In another embodiment, the kit comprises one or more primer sets capable of detecting at least one or more of the following human microRNA target templates: miR-1, miR-7, miR-10b, miR-26a, miR-26b, miR-29a, miR-30e-3p, miR-95, miR-107, miR-141, miR-143, miR-154*, miR-154, miR-155, miR-181a, miR-181b, miR-181c, miR-190, miR-193, miR-194, miR-195, miR-202, miR-206, miR-208, miR-212, miR-221, miR-222, miR-224, miR-296, miR-299, miR-302c*, miR-302c, miR-320, miR-339, miR363, miR-376b, miR379, miR410, miR412, miR424, miR429, miR431, miR449, miR451, let7a, let7b, let7c, let7d, let7e, let7f, let7g, and let7i. Exemplary primers for use in accordance with this embodiment of the kit are provided in TABLE 1, TABLE 2 and TABLE 6 below.

In another embodiment, the kit comprises at least one oligonucleotide primer selected from the group consisting of SEQ ID NO: 2 to SEQ ID NO: 493, as shown in TABLE 1, TABLE 2, TABLE 6 and TABLE 7.

In another embodiment, the kit comprises at least one oligonucleotide primer selected from the group consisting of SEQ ID NO: 47, 48, 49, 50, 55, 56, 81, 82, 83, 84, 91, 92, 103, 104, 123, 124, 145, 146, 193, 194, 197, 198, 221, 222, 223, 224, 225, 226, 227, 228, 229, 230, 239, 240, 247, 248, 253, 254, 255, 256, 257, 258, 277, 278, 285, 286, 287, 288, 293, 294, 301, 302, 309, 310, 311, 312, 315, 316, 317, 318, 319, 320, 333, 334, 335, 336, 337, 338, 359, 360, 369, 370, 389, 390, 393, 394, 405, 406, 407, 408, 415, 416, 419, 420, 421, 422, 425, 426, 429, 430, 431, 432, 433, 434, 435, 436, 437, 438, 439, 440, 441, 442, 443, 444, 461 and 462, as shown in TABLE 6.

A kit of the invention can also provide reagents for primer extension and amplification reactions. For example, in some embodiments, the kit may further include one or more of the following components: a reverse transcriptase enzyme, a DNA polymerase enzyme, a Tris buffer, a potassium salt (e.g., potassium chloride), a magnesium salt (e.g., magnesium chloride), a reducing agent (e.g., dithiothreitol), and deoxynucleoside triphosphates (dNTPs).

In various embodiments, the kit may include a detection reagent such as SYBR green dye or BEBO dye that preferentially or exclusively binds to double stranded DNA during a PCR amplification step. In other embodiments, the kit may include a forward and/or reverse primer that includes a fluorophore and quencher to measure the amount of the PCR amplification products.

The kit optionally includes instructions for using the kit in the detection and quantitation of one or more mammalian microRNA targets. The kit can also be optionally provided in a suitable housing that is preferably useful for robotic handling in a high throughput manner.

The following examples merely illustrate the best mode now contemplated for practicing the invention, but should not be construed to limit the invention.

### EXAMPLE 1

This Example describes a representative method of the invention for producing DNA molecules from microRNA target molecules.

Primer extension was conducted as follows (using InVitrogen SuperScript III® reverse transcriptase and following the guidelines that were provided with the enzyme). The following reaction mixture was prepared on ice:
1 μl of 10 mM dNTPs
1 μl of 2 μM extension primer
1-5 μl of target template
4 μL of “5× cDNA buffer”
1 μl of 0.1 M DTT
1 μl of RNAse OUT
1 μl of SuperScript III® enzyme
water to 20 μl

The mixture was incubated at 50° C. for 30 minutes, then 85° C. for 5 minutes, then cooled to room temperature and diluted 10-fold with TE (10 mM Tris, pH 7.6, 0.1 mM EDTA).

Real-time PCR was conducted using an ABI 7900 HTS detection system (Applied Biosystems, Foster City, Calif., U.S.A.) by monitoring SYBR® green fluorescence of double-stranded PCR amplicons as a function of PCR cycle number. A typical 10 μl PCR reaction mixture contained:
5 μl of 2×SYBR® green master mix (ABI)
0.8 μl of 10 μM universal forward primer
0.8 μl of 10 μM reverse primer
1.4 μl of water
2.0 μl of target template (10-fold diluted RT reaction).

The reaction was monitored through 40 cycles of standard “two cycle” PCR (95° C.-15 sec; 60° C.-60 sec) and the fluorescence of the PCR products was measured.

The foregoing method was successfully used in eleven primer extension PCR assays for quantitation of endogenous microRNAs present in a sample of total RNA. The DNA sequences of the extension primers, the universal forward primer sequence, and the LNA substituted reverse primers, used in these 11 assays are shown in TABLE 1.

TABLE 1

| | | | | |
|---|---|---|---|---|
| | Primer | | | SEQ ID |
| Target microRNA | number | Primer Name | DNA sequence (5′ to 3′) | NO |

gene-secific extension primers¹

| | | | | |
|---|---|---|---|---|
| humanb let7a | 357 | let7aP4 | CATGATCAGCTGGGCCAAGAAACTATACAACCT | 2 |
| human miR-1 | 337 | miR1P5 | CATGATCAGCTGGGCCAAGATACATACTTCT | 3 |
| human miR-15a | 344 | miR15aP3 | CATGATCAGCTGGGCCAAGACACAAACCATTATG | 4 |
| human miR-16 | 351 | miR16P2 | CATGATCAGCTGGGCCAAGACGCCAATATTTACGT | 5 |
| human miR-21 | 342 | miR21P6 | CATGATCAGCTGGGCCAAGATCAACATCAGT | 6 |
| human miR-24 | 350 | miR24P5 | CATGATCAGCTGGGCCAAGACTGTTCCTGCTG | 7 |
| human miR-122 | 222 | 122-E5F | CATGATCAGCTGGGCCAAGAACAAACACCATTGTCA | 8 |
| human miR-124 | 226 | 124-E5F | CATGATCAGCTGGGCCAAGATGGCATTCACCGCGTG | 9 |
| human miR-143 | 362 | miR143P5 | CATGATCAGCTGGGCCAAGATGAGCTACAGTG | 10 |
| human miR-145 | 305 | miR145P2 | CATGATCAGCTGGGCCAAGAAAGGGATTCCTGGGAA | 11 |
| human miR-155 | 367 | miR155P3 | CATGATCAGCTGGGCCAAGACCCCTATCACGAT | 12 |

universal forward primer

| | | | |
|---|---|---|---|
| 230 | E5F | CATGATCAGCTGGGCCAAGA | 13 |

RNA species-specific reverse primers²

| | | | | |
|---|---|---|---|---|
| human let7a | 290 | miRlet7a- | TG+AGGT+AGTAGGTTG | 14 |
| | | 1, 2, 3R | | |
| human miR-1 | 285 | miR1-1, 2R | TG+GAA+TG+TAAAGAAGTA | 15 |
| human miR-15a | 287 | miR15aR | TAG+CAG+CACATAATG | 16 |
| human miR-16 | 289 | miR16-1, 2R | T+AGC+AGCACGTAAA | 17 |
| human miR-21 | 286 | miR21R | T+AG+CT+TATCAGACTGAT | 18 |
| human miR-24 | 288 | miR24-1, 2R | TGG+CTCAGTTCAGC | 19 |
| human miR-122 | 234 | 122LNAR | T+G+GAG+TGTGACAA | 20 |
| human miR-124 | 235 | 124LNAR | T+TAA+GGCACGCG | 21 |
| human miR-143 | 291 | miR143R | TG+AGA+TGAAGCACTG | 22 |
| human miR-145 | 314 | miR145R2 | GT+CCAGTTTTCCCA | 23 |
| human miR-155 | 293 | miR155R | T+TAA+TG+CTAATCGTGA | 24 |

¹-Universal forward primer binding sites are shown in italics. The overlap with the RNA-specific reverse primers are underlined.
²-LNA molecules are preceded by a “+”. Region of overlap of the reverse primers with the corresponding extension primers are underlined.

The assay was capable of detecting microRNA in a concentration range of from 2 nM to 20 fM. The assays were linear at least up to a concentration of 2 nM of synthetic microRNA (>1,000,000 copies/cell).

### EXAMPLE 2

This Example describes the evaluation of the minimum sequence requirements for efficient primer-extension mediated cDNA synthesis using a series of extension primers for microRNA assays having gene specific regions that range in length from 12 to 3 base pairs.

Primer Extension Reactions: Primer extension was conducted using the target molecules miR-195 and miR-215 as follows. The target templates miR-195 and miR-215 were diluted to 1 nM RNA (100,000 copies/cell) in TE zero plus 100 ng/μl total yeast RNA. A no template control (NTC) was prepared with TE zero plus 100 ng/μl total yeast RNA.

The reverse transcriptase reactions were carried out as follows (using InVitrogen SuperScript III® reverse transcriptase and following the guidelines that were provided with the enzyme) using a series of extension primers for miR-195 (SEQ ID NO: 25-34) and a series of extension primers for miR-215 (SEQ ID NO: 35-44) the sequences of which are shown below in TABLE 2.

The following reaction mixtures were prepared on ice:

Set 1: No Template Control

37.5 μl water

12.5 μl of 10 mM dNTPs

12.5 μl 0.1 mM DTT

50 μl of “5× cDNA buffer”

12.5 μl RNAse OUT

12.5 μl Superscript III® reverse transcriptase enzyme

12.5 μl 1 μg/μl Hela cell total RNA (Ambion)

plus 50 μl of 2 μM extension primer

plus 50 μl TEzero+yeast RNA

Set 2: Spike-in Template

37.5 μl water

12.5 μl of 10 mM dNTPs

12.5 μl 0.1 mM DTT

50 μl of “5× cDNA buffer”

12.5 μl RNAse OUT

12.5 μl Superscript III® reverse transcriptase enzyme (InVitrogen)

12.5 μl 1 μg/μl Hela cell total RNA (Ambion)

plus 50 μl of 2 μM extension primer

plus 50 μl 1 nM RNA target template (miR-195 or miR-215) serially diluted in 10-fold increments

The reactions were incubated at 50° C. for 30 minutes, then 85° C. for 5 minutes, and cooled to 4° C. and diluted 10-fold with TE (10 mM Tris, pH 7.6, 0.1 mM EDTA).

Quantitative Real-Time PCR reactions: Following reverse transcription, quadruplicate measurements of cDNA were made by quantitative real-time (qPCR) using an ABI 7900 HTS detection system (Applied Biosystems, Foster City, Calif., U.S.A.) by monitoring SYBR® green fluorescence of double-stranded PCR amplicons as a function of PCR cycle number. The following reaction mixture was prepared:

5 μl of 2×SYBR green master mix (ABI)

0.8 μl of 10 μM universal forward primer (SEQ ID NO: 13)

0.8 μl of 10 μM reverse primer (miR-195RP:SEQ ID NO: 45 or miR215RP: SEQ ID NO: 46)

1.4 μl of water

2.0 μl of target template (10-fold diluted miR-195 or miR-215 RT reaction)

Quantitative real-time PCR was performed for each sample in quadruplicate, using the manufacturer's recommended conditions. The reactions were monitored through 40 cycles of standard “two cycle” PCR (95° C.-15 sec, 60° C.-60 sec) and the fluorescence of the PCR products were measured and disassociation curves were generated. The DNA sequences of the extension primers, the universal forward primer sequence, and the LNA substituted reverse primers, used in the miR-195 and miR-215 assays are shown below in TABLE 2. The assay results for miR-195 are shown below in TABLE 3 and the assay results for miR-215 are shown below in TABLE 4.

TABLE 2

| | | | | |
|---|---|---|---|---|
| | | | | SEQ |
| Target | Primer | Primer | | ID |
| microRNA | number | Name | DNA sequence (5 to 3′) | NO: |

gene-specific extension primers¹

| | | | | |
|---|---|---|---|---|
| miR-195 | 646 | mir195-GS1 | CATGATCAGCTGGGCCAAGAGCCAATATTTCT | 25 |
| miR-195 | 647 | mir195-GS2 | CATGATCAGCTGGGCCAAGAGCCAATATTTC | 26 |
| miR-195 | 648 | mir195-GS3 | CATGATCAGCTGGGCCAAGAGCCAATATTT | 27 |
| miR-195 | 649 | mir195-GS4 | CATGATCAGCTGGGCCAAGAGCCAATATT | 28 |
| miR-195 | 650 | mir195-GS5 | CATGATCAGCTGGGCCAAGAGCCAATAT | 29 |
| miR-195 | 651 | mir195-GS6 | CATGATCAGCTGGGCCAAGAGCCAATA | 30 |
| miR-195 | 652 | mir195-GS7 | CATGATCAGCTGGGCCAAGAGCCAAT | 31 |
| miR-195 | 653 | mir195-GS8 | CATGATCAGCTGGGCCAAGAGCCAA | 32 |
| miR-195 | 654 | mir195-GS9 | CATGATCAGCTGGGCCAAGAGCCA | 33 |
| miR-195 | 655 | mir195-GS10 | CATGATCAGCTGGGCCAAGAGCC | 34 |
| miR-215 | 656 | mir215-GS1 | CATGATCAGCTGGGCCAAGAGTCTGTCAATTC | 35 |
| miR-215 | 657 | mir215-GS2 | CATGATCAGCTGGGCCAAGAGTCTGTCAATT | 36 |
| miR-215 | 658 | mir215-GS3 | CATGATCAGCTGGGCCAAGAGTCTGTCAAT | 37 |
| miR-215 | 659 | mir215-GS4 | CATGATCAGCTGGGCCAAGAGTCTGTCAA | 38 |
| miR-215 | 660 | mir215-GS5 | CATGATCAGCTGGGCCAAGAGTCTGTCA | 39 |
| miR-215 | 661 | mir215-GS6 | CATGATCAGCTGGGCCAAGAGTCTGTC | 40 |
| miR-215 | 662 | mir215-GS7 | CATGATCAGCTGGGCCAAGAGTCTGT | 41 |
| miR-215 | 663 | mir215-GS8 | CATGATCAGCTGGGCCAAGAGTCTG | 42 |
| miR-215 | 664 | mir215-GS9 | CATGATCAGCTGGGCCAAGAGTCT | 43 |
| miR-215 | 665 | mir215-GS10 | CATGATCAGCTGGGCCAAGAGTC | 44 |

RNA species-specific reverse primers²

| | | | | |
|---|---|---|---|---|
| miR-195 | 442 | mir195RP | T+AGC+AGCACAGAAAT | 45 |
| miR-215 | 446 | mir215RP | AT+GA+CCTATGAATTG | 46 |

¹-Universal forward primer binding sites are shown in italics.
²- The “+” symbol precedes the LNA molecules.

Results:

The sensitivity of each assay was measured by the cycle threshold (Ct) value which is defined as the cycle count at which fluorescence was detected in an assay containing microRNA target template. The lower this Ct value (e.g. the fewer number of cycles), the more sensitive was the assay. For microRNA samples, it was generally observed that while samples that contain template and no template controls both eventually cross the detection threshold, the samples with template do so at a much lower cycle number. The ΔCt value is the difference between the number of cycles (Ct) between template containing samples and no template controls, and serves as a measure of the dynamic range of the assay. Assays with a high dynamic range allow measurements of very low microRNA copy numbers. Accordingly, desirable characteristics of a microRNA detection assay include high sensitivity (low Ct value) and broad dynamic range (ΔCt≧12) between the signal of a sample containing target template and a no template background control sample.

The results of the miR195 and miR215 assays using extension primers having a gene specific portion ranging in size from 12 nucleotides to 3 nucleotides are shown below in TABLE 3 and TABLE 4, respectively. The results of these experiments unexpectedly demonstrate that gene-specific priming sequences as short as 3 nucleotides exhibit template specific priming. For both the miR-195 assay sets (shown in TABLE 3) and the miR-215 assay sets (shown in TABLE 4), the results demonstrate that the dynamic range (ΔCt) for both sets of assays are fairly consistent for extension primers having gene specific regions that are greater or equal to 8 nucleotides in length. The dynamic range of the assay (ΔCt) begins to decrease for extension primers having gene specific regions below 8 nucleotides, with a reduction in assay specificity below 7 nucleotides in the miR-195 assays, and below 6 nucleotides in the miR-215 assays. A melting point analysis of the miR-215 samples demonstrated that even at 3 nucleotides, there is specific PCR product present in the plus template samples (data not shown). Taken together, these data demonstrate that the gene specific region of extension primers is ideally ≧8 nucleotides, but can be as short as 3 nucleotides in length.

TABLE 3
miR195 Assay Results

| | Ct: No Template | | |
|---|---|---|---|
| GS Primer Length | Control | Ct: Plus Template | Δ Ct |
| 12 | 34.83 | 20.00 | 14.82 |
| 12 | 34.19 | 19.9 | 14.3 |
| 11 | 40.0 | 19.8 | 20.2 |
| 10 | 36.45 | 21.2 | 15.2 |
| 9 | 36.40 | 22.2 | 14.2 |
| 8 | 40.0 | 23.73 | 16.27 |
| 7 | 36.70 | 25.96 | 10.73 |
| 6 | 30.95 | 26.58 | 4.37 |
| 5 | 30.98 | 31.71 | −0.732 |
| 4 | 32.92 | 33.28 | −0.364 |
| 3 | 35.98 | 35.38 | −0.605 |

Ct = the cycle count where the fluorescence exceeds the threshold of detection. ΔCt = the difference between the Ct value with template and no template.

TABLE 4
miR215 Assay Results

| | Ct: No Template | | |
|---|---|---|---|
| GS Primer Length | Control | Ct: Plus Template | Δ Ct |
| 12 | 33.4 | 13.57 | 19.83 |
| 12 | 33.93 | 14.15 | 19.77 |
| 11 | 35.51 | 15.76 | 19.75 |
| 10 | 35.33 | 15.49 | 19.84 |
| 9 | 36.02 | 16.84 | 19.18 |
| 8 | 35.79 | 17.07 | 18.72 |
| 7 | 32.29 | 17.58 | 14.71 |
| 6 | 34.38 | 20.62 | 13.75 |
| 5 | 34.41 | 28.65 | 5.75 |
| 4 | 36.36 | 33.92 | 2.44 |
| 3 | 35.09 | 33.38 | 1.70 |

Ct = the cycle count where the fluorescence exceeds the threshold of detection. ΔCt = the difference between the Ct value with template and no template.

### EXAMPLE 3

This Example describes assays and primer sets designed for quantitative analysis of human microRNA expression patterns.

Primer Design:

microRNA target templates: the sequence of the target templates as described herein are publicly available accessible on the World Wide Web at the Welcome Trust Sanger Institute website in the “miRBase sequence database” as described in Griffith-Jones et al. (2004), Nucleic Acids Research 32:D109-D111 and Griffith-Jones et al. (2006) Nucleic Acids Research 34: D140-D144.

Extension primers: gene specific primers for primer extension of a microRNA to form a cDNA followed by quantitative PCR (qPCR) amplification were designed to (1) convert the RNA template into cDNA; (2) to introduce a “universal” PCR binding site (SEQ ID NO:1) to one end of the cDNA molecule; and (3) to extend the length of the cDNA to facilitate subsequent monitoring by qPCR.

Reverse primers: unmodified reverse primers and locked nucleic acid (LNA) containing reverse primers (RP) were designed to quantify the primer-extended, full length cDNA in combination with a generic universal forward primer (SEQ ID NO:13). For the locked nucleic acid containing reverse primers, two or three LNA modified bases were substituted within the first 8 nucleotides from the 5′ end of the reverse primer oligonucleotide, as shown below in the exemplary reverse primer sequences provided in TABLE 6. The LNA base substitutions were selected to raise the predicted Tm of the primer by the highest amount, and the final predicted Tm of the selected primers were specified to be preferably less than or equal to 55° C.

An example describing an assay utilizing an exemplary set of primers the detection of miR-95 and miR-424 is described below.

Primer Extension Reactions: primer extension was conducted using DNA templates corresponding to miR-95 and miR-424 as follows. The DNA templates were diluted to 0 nM, 1 nM, 100 pM, 10 pM and 1 pM dilutions in TE zero (10 mM Tris pH7.6, 0.1 mM EDTA) plus 100 ng/μl yeast total RNA (Ambion, Austin Tex.).

The reverse transcriptase reactions were carried out using the following primers:

Extension primers: (diluted to 500 nM)

(SEQ ID NO:123)

| | |
|---|---|
| miR-95GSP | CATGATCAGCTGGGCCAAGATGCTCAATAA |

(SEQ ID NO:415)

| | |
|---|---|
| miR-424GSP | CATGATCAGCTGGGCCAAGATTCAAAACAT |

Reverse primers: (diluted to 10 mM).

(SEQ ID NO:124)

| | |
|---|---|
| miR-95_RP4 | TT+CAAC+GGGTATTTATTGA |

(SEQ ID NO:416)

| | |
|---|---|
| miR-424RP2 | C+AG+CAGCAATTCATGTTTT |

Reverse Transcription (Per Reaction):

2 μl water

2 μl of “5× cDNA buffer” (InVitrogen, Carlsbad, Calif.)

0.5 μl of 0.1 mM DTT (InVitrogen, Carlsbad, Calif.)

0.5 μl of 10 mM dNTPs (InVitrogen, Carlsbad, Calif.)

0.5 μl RNAse OUT (InVitrogen, Carlsbad, Calif.)

0.5 μl Superscript III® reverse transcriptase enzyme (InVitrogen, Carlsbad, Calif.)

2 μl of extension primer plus 2 μl of template dilution.

The reactions were mixed and incubated at 50° C. for 30 minutes, then 85° C. for 5 minutes, and cooled to 4° C. and diluted 10-fold with TE zero.

Quantitative Real-Time PCR Reactions: (per reaction)

5 μl 2×SYBR mix (Applied Biosystems, Foster City, Calif.)

1.4p water

0.8 μl universal primer (CATGATCAGCTGGGCCAAGA (SEQ ID NO: 13))

2.0 μl of diluted reverse transcription (RT) product from above.

Quantitative real-time PCR was performed for each sample in quadruplicate, using the manufacturer's recommended conditions. The reactions were monitored through 40 cycles of standard “two cycle” PCR (95° C.-15 sec, 60° C.-60 sec) and the fluorescence of the PCR products were measured and disassociation curves were generated. The DNA sequences of the extension primers, the universal forward primer sequence, and the LNA substituted reverse primers, used in the representative miR-95 and miR-424 assays as well as primer sets for 212 different human microRNA templates are shown below in TABLE 6. Primer sets for assays requiring extensive testing and design modification to achieve a sensitive assay with a high dynamic range are indicated in TABLE 6 with the symbol # following the primer name.

Results:

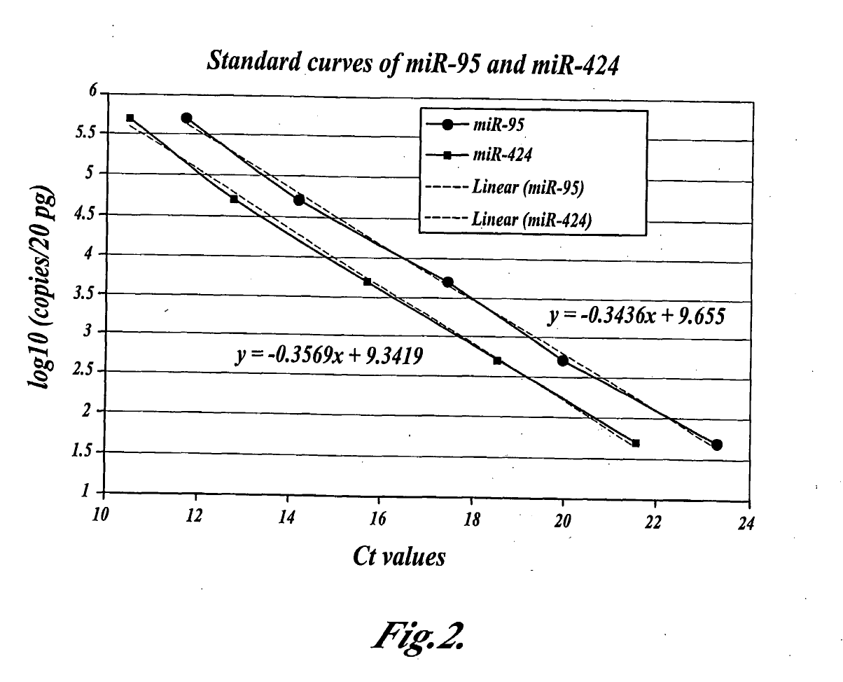

TABLE 5 shows the Ct values (averaged from four samples) from the miR-95 and miR-424 assays, which are plotted in the graph shown in FIG. 2. The results of these assays are provided as representative examples in order to explain the significance of the assay parameters shown in TABLE 6 designated as slope (column 6), intercept (column 7) and background (column 8).

As shown in TABLE 5, the Ct value for each template at various concentrations is provided. The Ct values (x-axis) are plotted as a function of template concentration (y-axis) to generate a standard curve for each assay, as shown in FIG. 2. The slope and intercept define the assay measurement characteristics that permit an estimation of number of copies/cell for each microRNA. For example, when the Ct values for 50 μg total RNA input for the miR-95 assay are plotted, a standard curve is generated with a slope and intercept of −0.03569 and 9.655, respectively. When these standard curve parameters are applied to the Ct of an unknown sample (x), they yield log 10 (copies/20 pg total RNA) (y). Because the average cell yields 20 pg of total RNA, these measurements equate to copies of microRNA/cell. The background provides an estimate of the minimum copy number that can be measured in a sample and is computed by inserting the no template control (NTC) value into this equation. In this example, as shown in TABLE 6, miR-95 yields a background of 1.68 copies/20 pg at 50 μg of RNA input.

As further shown in TABLE 6, reverse primers that do not contain LNA may also be used in accordance with the methods of the invention. See, e.g. SEQ ID NO: 494-499. The sensitivity and dynamic range of the assays using non-LNA containing reverse primers SEQ ID NO: 494-499, yielded similar results to the corresponding assays using LNA-containing reverse primers.

TABLE 5
Ct Values (averaged from four samples)

| | Template concentration | | | | | |
|---|---|---|---|---|---|---|
| | 10 nM | 1 nM | 0.1 nM | 0.01 nM | 0.001 nM | NTC |
| copies/20 pg RNA | 500,000 | 50,000 | 5000 | 500 | 50 | |
| (50 μg input) | | | | | | |
| copies/20 pg RNA | 5,000,000 | 500,000 | 50,000 | 5000 | 500 | |
| (5 μg input) | | | | | | |
| miR-95 | 11.71572163 | 14.17978 | 17.46353 | 19.97259 | 23.33171 | 27.44383 |
| miR-424 | 10.47708975 | 12.76806 | 15.69251 | 18.53729 | 21.56897 | 23.2813 |
| log10 (copies for | 5.698970004 | 4.69897 | 3.69897 | 2.69897 | 1.69897 | |
| 50 μg input) | | | | | | |

TABLE 6
Primers to detect human microRNA target templates

| | | | | | | | |
|---|---|---|---|---|---|---|---|
| Human | | | | | | | |
| Target | | | Reverse | | | | |
| micro | Extension | Extension | Primer | Reverse | | Background | RNA input |
| RNA | Primer Name | Primer Sequence | Name | Primer Sequence | Slope | Intercept | 50 ug 5 ug |

| | | | | | | | | |
|---|---|---|---|---|---|---|---|---|
| miR-1 | miR1GSP10# | CATGATCAGCTGGGCCAA | miR-1RP# | T+G+GAA+TG+TAAAGAA | −0.2758 | 8.3225 | 2.44 | 24.36 |
| | | GATACATACTTC | | GT | | | | |
| | | SEQ ID NO:47 | | SEQ ID NO:48 | | | | |
| miR-7 | miR-7GSP # | CATGATCAGCTGGGCCAA | miR-7_RP6# | T+GGAA+GACTAGTGATT | −0.2982 | 10.435 | 11.70 | 116.99 |
| | | GACAACAAAATC | | TT | | | | |
| | | SEQ ID NO:49 | | SEQ ID NO:50 | | | | |
| miR-9* | miR-9*GSP | CATGATCAGCTGGGCCAA | miR-9*RP | TAAA+GCT+AGATAACCG | −0.2405 | 8.9145 | 3.71 | 37.15 |
| | | GAACTTTCGGTT | | SEQ ID NO:52 | | | | |
| | | SEQ ID NO:51 | | | | | | |
| miR-10a | miR-10aGSP | CATGATCAGCTGGGCCAA | miR-10aRP | T+AC+CCTGTAGATCCG | −0.2755 | 8.6976 | 0.09 | 0.94 |
| | | GACACAAATTCG | | SEQ ID NO:54 | | | | |
| | | SEQ ID NO:53 | | | | | | |
| miR-10b | miR- | CATGATCAGCTGGGGCAA | miR- | TA+CCC+TGT+AGAACCG | −0.3505 | 8.7109 | 0.55 | 5.52 |
| | 10b_GSP11# | GAACAAATTCGGT | 10b_RP2# | A | | | | |
| | | SEQ ID NO:55 | | SEQ ID NO:56 | | | | |
| miR-15a | miR-15aGSP | CATGATCAGCTGGGCCAA | miR-15aRP | T+AG+CAGCACATAAT | −0.2831 | 8.4519 | 4.40 | 44.01 |
| | | GACACAAACCAT | | SEQ ID NO:58 | | | | |
| | | SEQ ID NO:57 | | | | | | |
| miR-15b | miR-15bGSP2 | CATGATCAGCTGGGCCAA | miR-15bRP | T+AG+CAGCACATCAT | −0.2903 | 8.4206 | 0.18 | 1.84 |
| | | GATGTAAACCA | | SEQ ID NO:60 | | | | |
| | | SEQ ID NO:59 | | | | | | |
| miR-16 | miR-16GSP2 | CATGATCAGCTGGGCCAA | miR-16RP | T+AG+CAGCACGTAAA | −0.2542 | 9.3689 | 1.64 | 16.42 |
| | | GACGCCAATAT | | SEQ ID NO:62 | | | | |
| | | SEQ ID NO:61 | | | | | | |
| miR-17- | miR-17-3pGSP | CATGATCAGCTGGGCCAA | miR-17-3pRP | A+CT+GCAGTGAAGGG | −0.2972 | 8.2625 | 1.08 | 10.78 |
| | | GAACAAGTGCCT | | SEQ ID NO:64 | | | | |
| | | SEQ ID NO:63 | | | | | | |
| miR-17- | miR-17- | CATGATCAGCTGGGCCAA | miR-17-5pRP | C+AA+AGTGCTTAGAGTG | −0.2956 | 7.9101 | 0.13 | 1.32 |
| 5p | 5pGSP2 | GAACTACCTGC | | SEQ ID NO:66 | | | | |
| | | SEQ ID NO:65 | | | | | | |
| miR-19a | miR-19aGSP2 | CATGATCAGCTGGGCCA | miR-19aRP | TG+TG+CAAATCTATGG | −0.2984 | 9.461 | 0.02 | 0.23 |
| | | AGATCAGTTTTG | | SEQ ID NO:68 | | | | |
| | | SEQ ID NO:67 | | | | | | |
| miR-19b | miR-19bGSP | CATGATCAGCTGGGCCA | miR-19bRP | TG+TG+CAAATGCATG | −0.294 | 8.1434 | 2.26 | 22.55 |
| | | AGATCAGTTTTGC | | SEQ ID NO:70 | | | | |
| | | SEQ ID NO:69 | | | | | | |
| miR-20 | miR-20GSP3 | CATGATCAGCTGGGCCA | miR-20RP | T+AA+AGTGCTTATAGTG | −0.2979 | 7.9929 | 0.16 | 1.60 |
| | | AGACTACCTGC | | CA | | | | |
| | | SEQ ID NO:71 | | SEQ ID NO:72 | | | | |
| miR-21 | miR-21GsP2 | CATGATCAGGTGGGCCAA | miR-21RP | T+AG+CTTATCAGACTGA | −0.2849 | 8.1624 | 1.80 | 17.99 |
| | | GATCAACATCA | | TG | | | | |
| | | SEQ ID NO: 73 | | SEQ ID NO:74 | | | | |
| miR-23a | miR-23aGSP | CATGATCAGCTGGGCCA | miR-23aRP | A+TC+ACATTGCCAGG | −0.3172 | 9.4253 | 2.41 | 24.08 |
| | | AGAGGAAATCCCT | | SEQ ID NO:76 | | | | |
| | | SEQ ID NO:75 | | | | | | |
| miR-23b | miR-23bGSP | CATGATCAGCTGGGCCA | miR-23bRP | A+TG+ACATTGCCAGG | −0.2944 | 9.0985 | 5.39 | 53.85 |
| | | AGAGGTAATCCCT | | SEQ ID NO:78 | | | | |
| | | SEQ ID NO:77 | | | | | | |
| miR-25 | miR-25GSP | CATGATCAGCTGGGCCA | miR-25RP | C+AT+TGCACTTGTCTC | −0.3009 | 0.2482 | 1.52 | 15.19 |
| | | AGATCAGACCGAG | | SEQ ID NO:80 | | | | |
| | | SEQ ID NO:79 | | | | | | |
| miR-26a | miR-26aGSP9# | CATGATCAGCTGGGCCA | miR- | TT+CA+AGTAATCCAGGA | −0.2807 | 8.558 | 0.26 | 2.56 |
| | | AGAGCCTATCCT | 26aRP# | T | | | | |
| | | SEQ ID NO:81 | | SEQ ID NO:82 | | | | |
| miR-26b | miR-26bGSP9# | CATGATCAGCTGGGCCA | miR- | TT+CA+AGT+AATTCAGG | −0.2831 | 8.7885 | 0.37 | 3.67 |
| | | AGAAACCTATCC | 26bPR2# | AT | | | | |
| | | SEQ ID NO:83 | | SEQ ID NO:84 | | | | |
| miR-27a | miR-27aGSP | CATGATCAGCTGGGCCA | miR-27aRP | TT+CA+CAGTGGCTAA | −0.2765 | 9.5239 | 5.15 | 51.51 |
| | | AGAGCGGAACTTA | | SEQ ID NO:86 | | | | |
| | | SEQ ID NO:85 | | | | | | |
| miR-27b | miR-27bGSP | CATGATCAGCTGGGCCA | miR-27bRP | TT+CA+CAGTGGCTAA | −0.28 | 9.5483 | 5.97 | 59.71 |
| | | AGAGCAGAACTTA | | SEQ ID NO:88 | | | | |
| | | SEQ ID NO:87 | | | | | | |
| miR-28 | miR-28GSP | CATGATCAGCTGGGCCA | miR-28RP | A+AG+GAGCTCACAGT | −0.3226 | 10.071 | 7.19 | 71.87 |
| | | AGACTCAATAGAC | | SEQ ID NO:90 | | | | |
| | | SEQ ID NO:89 | | | | | | |
| miR-29a | miR-29aGSP8# | CATGATCAGCTGGGCCA | miR- | T+AG+CACCATCTGAAAT | −0.29 | 8.8731 | 0.04 | 0.38 |
| | | AGAAACCGATT | 29aRP# | SEQ ID NO:92 | | | | |
| | | SEQ ID NO:91 | | | | | | |
| miR-29b | miR-29bGSP2 | CATGATGAGCTGGGCCA | miR-29bRP2 | T+AG+CACCATTTGAAAT | −0.3162 | 9.6276. | 3.56 | 35.57 |
| | | AGAAACACTGAT | | CAG | | | | |
| | | SEQ ID NO:93 | | SEQ ID NO:94 | | | | |
| miR-30a- | miR-30a- | CATGATCAGCTGGGCCA | miR-30a- | T+GT+AAACATCCTCGAC | −0.2772 | 9.0694 | 1.92 | 19.16 |
| 5p | 5pGSP | AGACTTCCAGTCG | 5pRP | SEQ ID NO:96 | | | | |
| | | SEQ ID NO:95 | | | | | | |
| miR-30b | miR-30bGSP | CATGATCAGCTGGGCCA | miR-30bRP | TGT+AAA+GATCCTACAC | −0.2621 | 8.5974 | 0.11 | 1.13 |
| | | AGAAGCTGAGTGT | | T | | | | |
| | | SEQ ID NO:97 | | SEQ ID NO:98 | | | | |
| miR-30c | miR-30cGSP | CATGATCAGCTGGGCCA | miR-30cRP | TGT+AAA+CATCCTACAC | −0.2703 | 8.699 | 0.15 | 1.48 |
| | | AGAGCTGAGAGTG | | T | | | | |
| | | SEQ ID NO:99 | | SEQ ID NO:100 | | | | |
| miR-30d | miR-30dGSP | CATGATCAGCTGGGCCA | miR-30dRP | T+GTAAA+CATCCCCG | −0.2506 | 9.3875 | 0.23 | 2.31 |
| | | AGACTTCCAGTCG | | SEQ ID NO:102 | | | | |
| | | SEQ ID NO:101 | | | | | | |
| miR-30e- | miR-30e- | CATGATCAGCTGGGCCA | miR-30e- | CTTT+CAGT+CGGATGT | −0.325 | 11.144 | 6.37 | 63.70 |
| 3p | GSP9# | AGAGCTGTAAAC | 3pRP5# | TT | | | | |
| | | SEQ ID NO: 103 | | SEQ ID NO:104 | | | | |
| miR-30e- | miR-30e- | CATGATCAGCTGGGCCA | miR-30e- | TG+TAAA+CATCCTTGAC | −0.2732 | 8.1604 | 8.50 | 85.03 |
| 5p | GSP | AGATCCAGTCAAG | 5pRY | SEQ ID NO:106 | | | | |
| | | SEQ ID NO:105 | | | | | | |
| miR-31 | miR-31GSP | CATGATCAGCTGGGCCA | miR-31RP | G+GC+AAGATGCTGGC | −0.3068 | 8.2605 | 3.74 | 37.43 |
| | | AGACAGCTATGCC | | SEQ ID NO:108 | | | | |
| | | SEQ ID NO:107 | | | | | | |
| miR-32 | miR-32GSP | CATGATCAGCTGGGCCA | miR-32RP | TATTG+CA+CATTACTAA | −0.2785 | 8.958 | 0.39 | 3.93 |
| | | AGAGCAAGTTAGT | | G | | | | |
| | | SEQ ID NO:109 | | SEQ ID NO:110 | | | | |
| miR-33 | miR-33GSP2 | CATGATCAGCTGGGCCA | miR-33RP | G+TG+GATTGTAGTTGC | −0.3031 | 8.42 | 2.81 | 28.14 |
| | | AGACAATGCAAC | | SEQ ID NO:112 | | | | |
| | | SEQ ID NO:111 | | | | | | |
| miR-34a | miR-34aGSP | CATGATGAGCTGGGCCA | miR-34aRP | T+GG+CAGTGTCTTAG | −0.3062 | 9.1522 | 2.40 | 23.99 |
| | | AGAAACAACCAGC | | SEQ ID NO:114 | | | | |
| | | SEQ ID NO:113 | | | | | | |
| miR-34b | miR-34bGSP | CATGATCAGCTGGGCCA | miR-34bRP | TA+GG+CAGTGTCATT | −0.3208 | 9.054 . | 0.04 | 0.37 |
| | | AGACAATCAGCTA | | SEQ ID NO:116 | | | | |
| | | SEQ ID NO:115 | | | | | | |
| miR-34c | miR-34cGSP | CATGATCAGCTGGGCCA | miR-34cRP | A+GG+CAGTGTAGTTA | −0.2995 | 10.14 | 1.08. | 10.83 |
| | | AGAGCAATCAGCT | | SEQ ID NO:118 | | | | |
| | | SEQ ID NO:117 | | | | | | |
| miR-92 | miR-92GSP | CATGATCAGCTGGGCCA | miR-92RP | T+AT+TGCACTTGTCCC | −0.3012 | 8.6908 | 8.92 | 89.17 |
| | | AGACAGGCCGGGA | | SEQ ID NO:120 | | | | |
| | | SEQ ID NO:119 | | | | | | |
| miR-93 | miR-93GSP | CATGATCAGCTGGGCCA | miR-93RP | AA+AG+TGCTGTTCGT | −0.3025 | 7.9933 | 4.63 | 46.30 |
| | | AGACTACCTGCAC | | SEQ ID NO:122 | | | | |
| | | SEQ ID NO:121 | | | | | | |
| miR-95 | miR-95GSP# | CATGATCAGCTGGGCCAA | miR- | TT+CAAC+GGGTATTTAT | −0.3436 | 9.655 | 1.68 | 16.80 |
| | | GATGCTCAATAA | 95_RP4# | TGA | | | | |
| | | SEQ ID NO:123 | | SEQ ID NO:124 | | | | |
| miR-96 | miR-96GSP | CATGATCAGCTGGGCCAA | miR-96RP | T+TT+GGCACTAGCAG | −0.2968 | 9.2611 | 0.00 | 0.05 |
| | | GAGCAAAAATGT | | SEQ ID NO:126 | | | | |
| | | SEQ ID NO:125 | | | | | | |
| miR-98 | miR-98GSP | CATGATCAGCTGGGCCAA | miR-98RP | TGA+GGT+AGTAAGTTG | −0.2797 | 9.5654 | 1.05 | 10.48 |
| | | GACTAATACAA | | SEQ ID NO:128 | | | | |
| | | SEQ ID NO:127 | | | | | | |
| miR-99a | miR-99aGSP | CATGATCAGCTGGGCCAA | miR-99aRP | A+AC+CCGTAGATCGG | −0.2768 | 8.781 | 0.21 | 2.08 |
| | | GACAGAAGATCG | | SEQ ID NO:130 | | | | |
| | | SEQ ID NO:129 | | | | | | |
| miR-99b | miR-99bGSP | CATGATCAGCTGGGCCAA | miR-99bRP | C+AC+CCGTAGAACCG | −0.2747 | 7.9855 | 0.25 | 2.53 |
| | | GACGCAAGGTCG | | SEQ ID NO:132 | | | | |
| | | SEQ ID NO:131 | | | | | | |
| miR-100 | miR-100GSP | CATGATCAGCTGGGCCAA | miR-100RP | A+AG+CCGTAGATCCG | −0.2902 | 8.669 | 0.04 | 0.35 |
| | | GACACAAGTTCG | | SEQ ID NO:134 | | | | |
| | | SEQ ID NO:133 | | | | | | |
| miR-101 | miR-101GSP | CATGATCAGCTGGGCCAA | miR-101RP | TA+CAG+TACTGTGATAA | −0.3023 | 8.2976 | 0.46 | 4.63 |
| | | GACTTCAGTTAT | | CT | | | | |
| | | SEQ ID NO:135 | | SEQ ID NO:136 | | | | |
| miR-103 | miR-103GSP | CATGATCAGCTGGGCCAA | miR-103RP | A+GC+AGCATTGTACA | −0.3107 | 8.5776 | 0.02 | 0.21 |
| | | GATCATAGCCCT | | SEQ ID NO:138 | | | | |
| | | SEQ ID NO:137 | | | | | | |
| miR-105 | miR-105GSP | CATGATCAGCTGGGCCAA | miR-105RP | T+CAAA+TGCTCAGACT | −0.2667 | 8.9832 | 0.93 | 9.28 |
| | | GAACAGGAGTCT | | SEQ ID NO:140 | | | | |
| | | SEQ ID NO:139 | | | | | | |
| miR-106a | miR-106aGSP | CATGATCAGCTGGGCCAA | miR-106aRP | AAA+AG+TGCTTACAGTG | −0.3107 | 8.358 | 0.03 | 0.31 |
| | | GAGCTACCTGCA | | SEQ ID NO:142 | | | | |
| | | SEQ ID NO:141 | | | | | | |
| miR-106b | miR-106bGSP | CATGATCAGCTGGGCCAA | miR-106bRP | T+AAAG+TGCTGACAGT | −0.2978 | 8.7838 | 0.10 | 1.04 |
| | | GAATCTGCACTG | | SEQ ID NO:144 | | | | |
| | | SEQ ID NO:143 | | | | | | |
| miR-107 | miR107GSP8# | CATGATCAGCTGGGCCAA | miR- | A+GC+AGCATTGTACAG | −0.304 | 9.1666 | 0.34 | 3.41 |
| | | GATGATAGCC | 107RP2# | SEQ ID NO:146 | | | | |
| | | SEQ ID NO:145 | | | | | | |
| miR-122a | miR-122aGSP | CATGATCAGCTGGGCCAA | miR-122aRP | T+GG+AGTGTGACAAT | −0.3016 | 8.1479 | 0.06 | 0.58 |
| | | GAACAAACACCA | | SEQ ID NO:148 | | | | |
| | | SEQ ID NO:147 | | | | | | |
| miR-124a | miR-124aGSP | CATGATCAGCTGGGCCAA | miR-124aRP | T+TA+AGGCAGGCGGT | −0.3013 | 8.6906 | 0.56 | 5.63 |
| | | GATGGCATTCAC | | SEQ ID NO:150 | | | | |
| | | SEQ ID NO:149 | | | | | | |
| miR-125a | miR-125aGSP | CATGATCAGCTGGGCCAA | miR-125aRP | T+GC+GTGAGACCCTT | −0.2938 | 8.6754 | 0.09 | 0.91 |
| | | GACACAGGTTAA | | SEQ ID NO:152 | | | | |
| | | SEQ ID NO:151 | | | | | | |
| miR-125b | miR-125bGSP | CATGATCAGCTGGGCCAA | miR-125bRP | T+CC+CTGAGACCCTA | −0.283 | 8.1251 | 0.20 | 1.99 |
| | | GATCACAAGTTA | | SEQ ID NO:154 | | | | |
| | | SEQ ID NO:153 | | | | | | |
| miR-126 | miR-126GSP | CATGATCAGCTGGGCCAA | miR-126RP | T+CG+TACCGTGAGTA | −0.26 | 8.937 | 0.18 | 1.80 |
| | | GAGCATTATTAC | | SEQ ID NO:156 | | | | |
| | | SEQ ID NO:155 | | | | | | |
| miR-126* | miR-126*GSP3 | CATGATCAGCTGGGCCAA | miR-16*RP | C+ATT+ATTA+GTTTT | −0.2969 | 8.184 | 3.58 | 35.78 |
| | | GACGCGTACC | | GGTACG | | | | |
| | | SEQ ID NO:157 | | SEQ ID NO:158 | | | | |
| miR-127 | miR-127GSP | CATGATCAGCTGGGCCAA | miR-127RP | T+CG+GATCCGTCTGA | −0.2432 | 9.1013 | 1.11 | 11.13 |
| | | GAAGCCAAGCTC | | SEQ ID NO:160 | | | | |
| | | SEQ ID NO:159 | | | | | | |
| miR-128a | miR-128aGSP | CATGATCAGCTGGGCCAA | miR-128aRP | T+CA+CAGTGAACCGG | −0.2866 | 8.0867 | 0.16 | 1.60 |
| | | GAAAAAGAGACC | | SEQ ID NO:162 | | | | |
| | | SEQ ID NO:161 | | | | | | |
| miR-128b | miR-128bGSP | CATGATCAGCTGGGCCAA | miR-128bRP | T+CA+CAGTGAAGCGG | −0.2923 | 8.0608 | 0.07 | 0.74 |
| | | GAGAAAGAGACC | | SEQ ID NO:164 | | | | |
| | | SEQ ID NO:163 | | | | | | |
| miR-129 | miR-129GSP | CATGATCAGCTGGGCCAA | miR-129RP | CTTTTTG+CGGTCTG | −0.2942 | 9.7731 | 0.88 | 8.85 |
| | | GAGCAAGCCCAG | | SEQ ID NO:166 | | | | |
| | | SEQ ID NO:165 | | | | | | |
| miR-130a | miR-130aGSP | CATGATCAGCTGGGCCAA | miR-130aRP | C+AG+TGCAATGTTAAAA | −0.2943 | 8.7465 | 1.28 | 12.78 |
| | | GAATGCCCTTTT | | G | | | | |
| | | SEQ ID NO:167 | | SEQ ID NO:168 | | | | |
| miR-130b | miR-130hGSP | CATGATCAGCTGGGCCAA | miR-130bRP | C+AG+TGCAATGATGA | −0.2377 | 9.1403 | 3.14 | 31.44 |
| | | GAATGCCCTTTC | | SEQ ID NO:170 | | | | |
| | | SEQ ID NO:169 | | | | | | |
| miR-132 | miR-132GSP | CATGATCAGCTGGGCCAA | miR-132RP | T+AA+CAGTCTACAGCC | −0.2948 | 8.1167 | 0.11 | 1.13 |
| | | GACGACCATGGC | | SEQ ID NO:172 | | | | |
| | | SEQ ID NO:171 | | | | | | |
| miR-133a | miR-133aGSP | CATGATCAGCTGGGCCAA | miR-133aRP | T+TG+GTCCCCTTCAA | −0.295 | 9.3679 | 0.10 | 1.04 |
| | | GAACAGCTGGTT | | SEQ ID NO:174 | | | | |
| | | SEQ ID NO:173 | | | | | | |
| mmR-133b | miR-133bGSP | CATGATCAGCTGGGCCAA | miR-133bRP | T+TG+GTCCCCTTGAA | −0.3062 | 8.3649 | 0.02 | 0.18 |
| | | GATAGCTGGTTG | | SEQ ID NO:176 | | | | |
| | | SEQ ID NO:175 | | | | | | |
| miR-134 | miR-134GSP | CATGATCAGCTGGGCCAA | miR-134RP | T+GT+GACTGGTTGAC | −0.2965 | 9.0483 | 0.14 | 1.39 |
| | | GACCCTCTGGTC | | SEQ ID NO:178 | | | | |
| | | SEQ ID NO:177 | | | | | | |
| miR-135a | miR-135aGSP | CATGATCAGCTGGGCCAA | miR-135aRP | T+AT+GGCTTTTTATTCC | −0.2914 | 8.092 | 1.75 | 17.50 |
| | | GATCACATAGGA | | G | | | | |
| | | SEQ ID NO:179 | | SEQ ID NO:180 | | | | |
| miR-135b | miR-135bGSP | CATGATCAGCTGGGCCAA | miR-135bRP | T+AT+GGGTTTTCATTCC | −0.2962 | 7.8986 | 0.05 | 0.49 |
| | | GACACATAGGAA | | SEQ ID NO:182 | | | | |
| | | SEQ ID NO:181 | | | | | | |
| miR-136 | miR-136GSP | CATGATCAGCTGGGCCAA | miR-136RP | A+CT+CCATTTGTTTTGA | −0.3616 | 10.229 | 0.68 | 6.77 |
| | | GATCCATCATCA | | TG | | | | |
| | | SEQ ID NO:183 | | SEQ ID NO:184 | | | | |
| miR-137 | miR-137GSP | CATGATCAGCTGGGCCAA | miR-137RP | T+AT+TGCTTAAGAATAC | −0.2876 | 8.234 | 8.57 | 85.71 |
| | | GATCCATCATCA | | GC | | | | |
| | | SEQ ID NO:185 | | SEQ ID NO:186 | | | | |
| miR-138 | miR-138GSP2 | CATGATCAGCTGGGCCAA | miR-138RP | A+GC+TGGTGTTGTGA | −0.3023 | 9.0814 | 0.22 | 2.19 |
| | | GACGGCCTGAT | | SEQ ID NO:188 | | | | |
| | | SEQ ID NO:187 | | | | | | |
| miR-139 | miR-139GSP | CATGATCAGCTGGGCCAA | miR-139RP | T+CT+ACAGTGCACGT | −0.2983 | 8.1141 | 6.92 | 69.21 |
| | | GAAGACACGTGC | | SEQ ID NO:190 | | | | |
| | | SEQ ID NO:189 | | | | | | |
| miR-140 | miR-140GSP | CATGATCAGCTGGGCCAA | miR-140RP | A+GT+GGTTTTACCCT | −0.2312 | 8.3231 | 0.13 | 1.34 |
| | | GACTACCATAGG | | SEQ ID NO:192 | | | | |
| | | SEQ ID NO:191 | | | | | | |
| miR-141 | miR141GSP9# | CATGATCAGCTGGGCCAA | miR- | TAA+CAC+TGTCTGGTAA | −0.2805 | 9.6671 | 0.13 | 1.26 |
| | | GAGCATCTTTA | | 141RP2# | | | | |
| | | SEQ ID NO:193 | | SEQ ID NO:194 | | | | |
| miR-142- | miR-142- | CATGATCAGCTGGGCCAA | miR-142- | TGT+AG+TGTTTCCTACT | −0.2976 8.4046 | 0.03 | 0.27 | |
| 3p | GSP3 | GATCCATAAA | 3pRP | SEQ ID NO:196 | | | | |
| | | SEQ ID NO:195 | | | | | | |
| miR143 | miR-143GSP8# | CATGATCAGCTGGGCCAA | miR- | T+GA+GATGAAGCACTG | −0.3008 | 9.2675 | 0.37 | 3.71 |
| | | GATGAGCTAC | 143RP2# | SEQ ID NO:198 | | | | |
| | | SEQ ID NO:197 | | | | | | |
| miR-144 | miR-144GSP2 | CATGATCAGCTGGGCCAA | miR-144RP | TA+CA+GTAT+AGATGAT | −0.2407 | 9.4441 | 0.95 | 9.52 |
| | | GACTAGTACAT | | G | | | | |
| | | SEQ ID NO:199 | | SEQ ID NO:200 | | | | |
| miR-145 | miR-14SGSP2 | CATGATCAGCTGGGCCAA | miR-145RP | G+TC+CAGTTTTCCCA | −0.2937 | 8.0791 | 0.39 | 3.86 |
| | | GAAAGGGATTC | | SEQ ID NO:202 | | | | |
| | | SEQ ID NO:201 | | | | | | |
| miR-146 | miR-146GSP3 | CATGATCAGCTGGGCCAA | miR-146RP | T+GA+GAACTGAATTCC | −0.2861 | 8.8246 | 0.08 | 0.75 |
| | | GAAACCCATG | | A | | | | |
| | | SEQ ID NO:203 | | SEQ ID NO:204 | | | | |
| miR-147 | miR-147GSP | CATGATCAGCTGGGCCAA | miR-147RP | G+TGTGTGGAAATGC | −0.2989 | 8.8866 | 1.65 | 16.47 |
| | | GAGCAGAAGCAT | | SEQ ID NO:206 | | | | |
| | | SEQ ID NO:205 | | | | | | |
| miR-148a | miR-148aGSP2 | CATGATCAGCTGGGCCAA | miR- | T+CA+GTGCACTACAGAA | −0.2928 | 9.4654 | 1.27 | 12.65 |
| | | GAACAAAGTTC | 148aRP2 | CT | | | | |
| | | SEQ ID NO:207 | | SEQ ID NO:208 | | | | |
| miR-148b | miR-148bGSP2 | CATGATCAGCTGGGCCAA | miR-148bRP | T+CA+GTGCATCACAG | −0.2982 | 10.417 | 0.24 | 2.44 |
| | | GAACAAAAGTTC | | SEQ ID NO:210 | | | | |
| | | SEQ ID NO:209 | | | | | | |
| miR-149 | miR-149GSP2 | CATGATCAGCTGGGCCAA | miR-149RP | T+GT+GGCTCCGTGTC | −0.2996 | 8.3392 | 2.15 | 21.50 |
| | | GAGGAGTGAAG | | SEQ ID NO:212 | | | | |
| | | SEQ ID NO:211 | | | | | | |
| miR-150 | miR-150GSP3 | CATGATCAGCTGGGCCAA | miR-150RP | T+CT+CGCAACCCTTG | −0.2943 | 8.3945 | 0.06 | 0.56 |
| | | GACACTGGTA | | SEQ ID NO:214 | | | | |
| | | SEQ ID NO:213 | | | | | | |
| miR-151 | miR-151GSP2 | CATGATCAGCTGGGCCAA | miR-151RP | A+CT+AGACTGAAGCTC | −0.2975 | 8.651 | 0.16 | 1.60 |
| | | GACCTCAAGGA | | SEQ ID NO:216 | | | | |
| | | SEQ ID NO:215 | | | | | | |
| miR-152 | miR-152GSP2 | CATGATCAGCTGGGCCAA | miR-152RP | T+CA+GTGCATGACAG | −0.2741 | 8.7404 | 0.33 | 3.25 |
| | | GACCCAAGTTC | | SEQ ID NO:218 | | | | |
| | | SEQ ID NO:217 | | | | | | |
| miR-153 | miR-153GSP2 | CATGATCAGCTGGGCCAA | miR-153RP | TTG+CAT+AGTCACAAAA | 0.2723 | 9.5732 | 3.32 | 33.19 |
| | | GATCACTTTTG | | SEQ ID NO:220 | | | | |
| | | SEQ ID NO:219 | | | | | | |
| miR-154* | miR- | CATGATGAGCTGGGCCAA | miR- | AATCA+TA+CACGGTTGA | −0.3056 | 8.8502 | 0.07 | 0.74 |
| | 154*GSP9# | GAAATAGGTCA | 154*RP2# | C | | | | |
| | | SEQ ID NO:221 | | SEQ ID NO:222 | | | | |
| miR-154 | miR-154GSP9# | CATGATCAGCTGGGCCAA | miR- | TA+GGTTA+TCCGTGTT | −0.3062 | 9.3947 | 0.10 | 0.96 |
| | | GACGAAGGCAA | 154RP3# | SEQ ID NO:224 | | | | |
| | | SEQ ID NO:223 | | | | | | |
| miR-155 | miR-155GSP8# | CATGATCAGCTGGGCCAA | miR- | TT+AA+TGCTAATCGTGA | −0.3201 | 8.474 | 5.49 | 54.91 |
| | | GACCCCTATC | 155RP2# | TAGG | | | | |
| | | SEQ ID NO:225 | | SEQ ID NO:226 | | | | |
| miR-181a | miR- | CATGATCAGCTGGGCCAA | miR- | AA+CATT+CAACGCTGTC | −0.2919 | 7.968 | 1.70 | 17.05 |
| | 181aGSP9# | GAACTCACCGA | 181aRP2# | SEQ ID N0:228 | | | | |
| | | SEQ ID NO:227 | | | | | | |
| miR-181c | miR- | CATGATCAGCTGGGCCAA | miR- | AA+GATT+CAACCTGTCG | −0.3102 | 7.9029 | 1.08 | 10.78 |
| | 181cGSP9# | GAACTCACCGA | 181cRP2# | SEQ ID NO:230 | | | | |
| | | SEQ ID NO:229 | | | | | | |
| miR-182* | miR-182*GSP | CATGATCAGCTGGGCCAA | miR-182*RP | T+GG+TTCTAGACTTGC | −0.2978 | 8.5876 | 4.25 | 42.47 |
| | | GATAGTTGGCAA | | SEQ ID NO:232 | | | | |
| | | SEQ ID NO:231 | | | | | | |
| miR-182 | miR-182GSP2 | CATGATCAGCTGGGCCAA | miR-182RP | TTT+GG+CAATGGTAG | −0.2863 | 9.0854 | 1.52 | 15.20 |
| | | GATGTGAGTTC | | SEQ ID NO:234 | | | | |
| | | SEQ ID NO:233 | | | | | | |
| miR-183 | miR-183GSP2 | CATGATCAGCTGGGCCAA | miR-183RP | T+AT+GGGACTGGTAG | −0.2774 | 9.9254 | 1.95 | 19.51 |
| | | GACAGTGAATT | | SEQ ID NO:236 | | | | |
| | | SEQ ID NO:235 | | | | | | |
| miR-184 | miR-184GSP2 | CATGATCAGCTGGGCCAA | miR-184RP | T+GG+ACGGAGAACTG | −0.2906 | 7.9585 | 0.05 | 0.49 |
| | | GAAACCCTTATC | | SEQ ID NO:238 | | | | |
| | | SEQ ID NO:237 | | | | | | |
| miR-186 | miR186GSP9# | CATGATCAGCTGGGCCAA | miR- | CA+AA+GAATT+CTCCTT | −0.2861 | 8.6152 | 0.32 | 3.18 |
| | | GAAAGCCCAAA | 186RP3# | TTGG | | | | |
| | | SEQ ID NO:239 | | SEQ ID NO:240 | | | | |
| miR-187 | miR-1870SP | CATGATCAGCTGGGCCAA | miR-187RP | T+CG+TGTCTTGTGTT | −0.2953 | 7.9329 | 1.23 | 12.31 |
| | | GACGGCTGCAAC | | SEQ ID NO:242 | | | | |
| | | SEQ ID NO:241 | | | | | | |
| miR-188 | miR-188GSP | CATGATCAGCTGGGCCAA | miR-188RP | C+AT+CCCTTGCATGG | −0.2925 | 8.0782 | 8.49 | 84.92 |
| | | GAACCCTCCACC | | SEQ ID NO:244 | | | | |
| | | SEQ ID NO:243 | | | | | | |
| miR-189 | miR-189GSP2 | CATGATCAGCTGGGCCAA | miR-189RP | G+TG+CCTACTGAGCT | −0.2981 | 8.8964 | 0.21 | 2.08 |
| | | GAACTGATATC | | SEQ ID NO:246 | | | | |
| | | SEQ ID NO:245 | | | | | | |
| miR-190 | miR1900SP9# | CATGATCAGCTGGGCCAA | miR- | T+GA+TA+TGTTTGATAT | −0.3317 | 9.8766 | 0.43 | 4.34 |
| | | GAACCTAATAT | 190RP4# | ATTAG | | | | |
| | | SEQ ID NO:247 | | SEQ ID NO:248 | | | | |
| miR-191 | miR-191GSP2 | CATGATCAGCTGGGCCAA | miR-191RP2 | C+AA+CGGAATCCCAAAA | −0.299 | 9.0317 | 0.41 | 4.07 |
| | | GAAGCTGCTTT | | G | | | | |
| | | SEQ ID NO:249 | | SEQ ID NO:250 | | | | |
| miR-192 | miR-192GSP2 | CATGATCAGCTGGGCCAA | miR-192RP | C+TGA+CCTATGAATTGA | −0.2924 | 9.5012 | 1.10 | 10.98 |
| | | GAGGCTGTCAA | | C | | | | |
| | | SEQ ID NO:251 | | SEQ ID NO:252 | | | | |
| miR-193 | miR-193GSP9# | CATGATCAGCTGGGCCAA | miR- | AA+CT+GGCCTACAAAG | −0.3183 | 8.9942 | 0.17 | 1.72 |
| | | GACTGGGACTT | 193RP2# | SEQ ID NO:254 | | | | |
| | | SEQ ID NO:253 | | | | | | |
| miR194 | mir194GSP8# | CATGATCAGCTGGGCCAA | mir194RP# | TG+TAA+GAGCAACTCCA | −0.3078 | 8.8045 | 0.37 | 3.69 |
| | | GATCCACATG | | SEQ ID NO:256 | | | | |
| | | SEQ ID NO:255 | | | | | | |
| miR-195 | miR-195GSP9# | CATGATCAGCTGGGCCAA | miR- | T+AG+CAG+CACAGAAAT | −0.2955 | 10.213 | 0.76 | 7.58 |
| | | GAGCCAATATT | 195RP3# | SEQ ID NO:258 | | | | |
| | | SEQ ID NO:257 | | | | | | |
| miR-196b | miR-196bGSP | CATGATCAGCTGGGCCAA | miR-196bRP | TA+GGT+AGTTTGGTGT | −0.301 | 8.1641 | 1.47 | 14.66 |
| | | GACCAACAACAG | | SEQ ID NO:260 | | | | |
| | | SEQ ID NO:259 | | | | | | |
| miR-196a | miR-196aGSP | CATGATCAGCTGGGCCAA | miR-196aRP | TA+GG+TAGTTTTCATGTT | −0.2932 | 8.0448 | 8.04 | 80.37 |
| | | GACCAACAACAT | | G | | | | |
| | | SEQ ID NO:261 | | SEQ ID NO:262 | | | | |
| miR-197 | miR-197GSP2 | CATGATCAGCTGGGCCAA | miR-197RP | TT+CA+CCACGTTGTC | −0.289 | 8.2822 | 0.71 | 7.10 |
| | | GAGCTGGGTGG | | SEQ ID NO:264 | | | | |
| | | SEQ ID NO:263 | | | | | | |
| miR-198 | miR-198GSP3 | CATGATCAGCTGGGCCAA | miR-198RP | G+GT+CCAGAGGGGAG | −0.2986 | 8.1359 | 0.31 | 3.15 |
| | | GACCTATCTC | | SEQ ID NO:266 | | | | |
| | | SEQ ID NO:265 | | | | | | |
| miR- | miR- | CATGATCAGCTGGGCCAA | miR- | T+AC+AGTAGTCTGCAC | −0.3029 | 9.0509 | 0.25 | 2.52 |
| 199a* | 199a*GSP2 | GAAACCAATGT | 199A*RP | SEQ ID NO:268 | | | | |
| | | SEQ ID NO:267 | | | | | | |
| miR-199a | miR-199aGSP2 | CATGATCAGCTGGGCCAA | miR-199aRP | C+CC+AGTGTTCAGAC | −0.3187 | 9.2268 | 0.12 | 1.16 |
| | | GAGAACAGGTA | | SEQ ID NO:270 | | | | |
| | | SEQ ID NO:269 | | | | | | |
| miR-199b | miR-199bGSP | CATGATCAGCTGGGCCAA | miR-199bRP | C+CC+AGTGTTTAGAC | −0.3165 | 9.3935 | 2.00 | 20.04 |
| | | GAGAACAGATAG | | SEQ ID NO:272 | | | | |
| | | SEQ ID NO:271 | | | | | | |
| miR-200a | miR-200aGSP2 | CATGATCAGCTGGGCCAA | miR-200aRP | TAA+CAC+TGTCTGGT | −0.2754 | 9.1227 | 0.08 | 0.78 |
| | | GAACATCGTTA | | SEQ ID NO:274 | | | | |
| | | SEQ ID NO:273 | | | | | | |
| miR-200b | miR-200bGSP2 | CATGATCAGCTGGGCCAA | miR-200bRP | TAATA+CTG+CCTGGTAA | −0.2935 | 8.5461 | 0.08 | 0.85 |
| | | GAGTCATCATT | | T | | | | |
| | | SEQ ID NO:275 | | SEQ ID NO:276 | | | | |
| miR-202 | miR-202 | CATGATCAGCTGGGCCAA | miR-202RP# | A+GA+GGTATA+GGGCAT | −0.2684 | 9.056 | 0.25 | 2.48 |
| | GSP10# | GATTTTCCCATG | | SEQ ID NO:278 | | | | |
| | | SEQ ID NO:277 | | | | | | |
| miR-203 | miR-203GSP2 | CATGATCAGCTGGGCCAA | miR-203RP | G+TG+AAATGTTTAGGAC | −0.2852 | 8.1279 | 1.60 | 16.03 |
| | | GACTAGTGGTC | | C | | | | |
| | | SEQ ID NO:279 | | SEQ ID NO:280 | | | | |
| miR-204 | miR-204GSP2 | CATGATCAGCTGGGCCAA | miR-204RP | T+TC+CCTTTGTCATCC | −0.2925 | 8.7648 | 0.16 | 1.59 |
| | | GAAGGCATAGG | | SEQ ID NO:282 | | | | |
| | | SEQ ID NO:281 | | | | | | |
| miR-205 | miR-205GSP | CATGATCAGCTGGGCCAA | miR-205RP | T+CCTT+CATTCCACC | −0.304 | 8.2407 | 9.21 | 92.15 |
| | | GACAGACTCCGG | | SEQ ID NO:284 | | | | |
| | | SEQ ID NO:283 | | | | | | |
| miR-206 | mir206GSP7# | CATGATCAGCTGGGCCAA | miR-206RP# | T+G+GAA+TGTAAGGAAG | −0.2815 | 8.2206 | 0.29 | 2.86 |
| | | GACCACACA | | TGT | | | | |
| | | SEQ ID NO:285 | | SEQ ID NO:286 | | | | |
| miR-208 | miR- | CATGATCAGCTGGGCCAA | miR- | ATAA+GA+CG+AGCAAAA | −0.2072 | 7.9097 | 57.75 | 577.52 |
| | 208_GsP13# | AACAAGCTTTTTGC | 208_RP4# | AG | | | | |
| | | SEQ ID NO:287 | | SEQ ID NO:288 | | | | |
| miR-210 | miR-210GSP | CATGATCAGCTGGGCCAA | miR-210RP | C+TG+TGCGTGTGACA | −0.2717 | 8.249 | 0.18 | 1.77 |
| | | GATCAGCCGCTG | | SEQ ID NO:290 | | | | |
| | | SEQ ID NO:289 | | | | | | |
| miR-211 | miR-211GSP2 | CATGATCAGCTGGGCCAA | miR-211RP | T+TG+CCTTTGTCATCC | −0.2926 | 8.3 106 | 0.10 | 1.00 |
| | | GAAGGCGAAGG | | SEQ ID NO:292 | | | | |
| | | SEQ ID NO:291 | | | | | | |
| miR-212 | miR-212GSP9# | CATGATCAGCTGGGCCAA | miR- | T+AA+CAGTCTCCAGTCA | -0,2916 | 8.0745 | 0.59 | 5.86 |
| | | GAGGCCGTGAC | 212RP2# | SEQ ID NO:294 | | | | |
| | | SEQ ID NO:293 | | | | | | |
| miR-213 | miR-213GSP | CATGATCAGCTGGGCCAA | miR-213RP | A+CC+ATCGACCGTTG | −0.2934 | 8.1848 | 2.96 | 29.59 |
| | | GAGGTACAATCA | | SEQ ID NO:296 | | | | |
| | | SEQ ID NO:295 | | | | | | |
| miR-214 | miR-214GSP | CATGATCAGCTGGGCCAA | miR-214RP | A+CA+GCAGGCACAGA | −0.2947 | 7.82 | 0.84 | 8.44 |
| | | GACTGCCTGTCT | | SEQ ID NO:298 | | | | |
| | | SEQ ID NO:297 | | | | | | |
| miR-215 | miR-215GSP2 | CATGATCAGCTGGGCCAA | miR-215RP | A+TGA+CCTATGAATTGA | −0.2932 | 8.9273 | 1.51 | 15.05 |
| | | GAGTCTGTCAA | | C | | | | |
| | | SEQ ID NO:299 | | SEQ ID NO:300 | | | | |
| miR216 | miR-216GSP9# | CATGATCAGCTGGGCCAA | mir216RP# | TAA+TCT+CAGCTGGCA | −0.273 | 8.5829 | 0.95 | 9.50 |
| | | GACACAGTTGC | | SEQ ID NO:302 | | | | |
| | | SEQ ID NO:301 | | | | | | |
| miR-217 | miR-217GSP2 | CATGATCAGCTGGGCCAA | miR-217RP2 | T+AC+TGCATCAGGAAGT | −0.3089 | 9.6502 | 0.07 | 0.71 |
| | | GAATCCAATCA | | GA | | | | |
| | | SEQ ID NO:303 | | SEQ ID NO:304 | | | | |
| miR-218 | mmR-218GSP2 | CATGATCAGCTGGGCCAA | miR-218RP | TTG+TGCTT+GATCTAAC | −0.2778 | 8.4363 | 1.00 | 10.05 |
| | | GAACATCATGGTTA | | SEQ ID NO:306 | | | | |
| | | SEQ ID NO:305 | | | | | | |
| miR-220 | miR-220GSP | CATGATCAGCTGGGCCAA | miR-220RP | C+CA+CACCGTATCTG | −0.2755 | 9.0728 | 8.88 | 88.75 |
| | | GAAAAGTGTCAG | | SEQ ID NO:308 | | | | |
| | | SEQ ID NO:307 | | | | | | |
| mir-221 | miR-221GSP9# | CATGATCAGCTGGGCCAA | miR-221RP# | A+GC+TACATTGTCTGC | −0.2886 | 8.5743 | 0.12 | 1.17 |
| | | GAGAAACCCAG | | SEQ ID NO:310 | | | | |
| | | SEQ ID NO:309 | | | | | | |
| miR-222 | miR-222GSP8# | CATGATCAGCTGGGCCAA | miR-222RP# | A+GC+TACATCTGGCT | −0.283 | 8.91 | 1.64 | 16.41 |
| | | GAGAGACCGA | | SEQ ID NO:312 | | | | |
| | | SEQ ID NO:311 | | | | | | |
| miR-223 | miR-223GSP | CATGATGAGCTGGGCCAA | miR-223RP | TG+TG+AGTTTGTCAAA | −0.2998 | 8.6669 | 0.94 | 9.44 |
| | | GAGGGGTATTTG | | SEQ ID NO:314 | | | | |
| | | SEQ ID NO:313 | | | | | | |
| miR-224 | miR-224GSP8# | CATGATCAGCTGGGCCAA | miR- | C+AAG+TCACTAGTGGTT | −0.2802 | 7.5575 | 0.56 | 5.63 |
| | | GATAAAACGGA | 224RP2# | SEQ ID NO:316 | | | | |
| | | SEQ ID NO:315 | | | | | | |
| miR-296 | miR-296GSP9# | CATGATCAGCTGGGCCAA | miR- | A+GG+GCCCCCCCTCAA | −0.3178 | 8.3856 | 0.10 | 0.96 |
| | | GAACAGGATTG | 296RP2# | SEQ ID NO:318 | | | | |
| | | SEQ ID NO:317 | | | | | | |
| miR-299 | miR-299GSP9# | CATGATCAGCTGGGCCAA | miR-299RP# | T+GG+TTTACCGTCCC | −0.3155 | 7.9383 | 1.30 | 12.96 |
| | | GTGTATGTG | | SEQ ID NO:320 | | | | |
| | | SEQ ID NO:319 | | | | | | |
| miR-301 | miR-301GSP | CATGATCAGCTGGGCCAA | miR-301RP | C+AG+TGCAATAGTATTT | −0.2839 | 8.314 | 2.55 | 25.52 |
| | | GAGCTTTGACAA | | GT | | | | |
| | | SEQ ID NO:321 | | SEQ ID NO:322 | | | | |
| miR- | miR-302a*GSP | CATGATCAGCTGGGCCAA | miR- | TAAA+CG+TGGATGTAC | −0.2608 | 8.392 | 10.04 | 0.41 |
| 302a* | | GAAAAGCAAGTA | 302a*RP | SEQ ID NO:324 | | | | |
| | | SEQ ID NO:323 | | | | | | |
| miR-302a | miR-302aGSP | CATGATCAGCTGGGCCAA | miR-302aRP | T+AAG+TGCTTCCATGT | −0.2577 | 9.6657 | 2.17 | 21.67 |
| | | GATCACCAAAAC | | SEQ ID NO:326 | | | | |
| | | SEQ ID NO:325 | | | | | | |
| miR- | mmR-302b*GSP | CATGATCAGCTGGGCCAA | miR- | A+CTTTAA+CATGGAAGT | −0.2702 | 8.5153 | 0.02 | 0.24 |
| 302b* | | GAAGAAAGCACT | 302b*RP | G | | | | |
| | | SEQ ID NO:327 | | SEQ ID NO:328 | | | | |
| miR-302b | miR-302bGSP | CATGATCAGCTGGGCCAA | miR-302bRP | T+AAG+TGCTTGCATGT | −0.2398 | 9.1459 | 5.11 | 51.11 |
| | | GACTACTAAAAC | | SEQ ID NO:330 | | | | |
| | | SEQ ID NO:329 | | | | | | |
| miR-302d | mmR-302dGSP | CATGATCAGCTGGGCCAA | miR-302dRP | T+AAG+TGCTTCCATGT | −0.2368 | 8.5602 | 5.98 | 59.78 |
| | | GAACACTCAAAC | | SEQ ID NO:332 | | | | |
| | | SEQ ID NO:331 | | | | | | |
| miR- | miR- | CATGATCAGCTGGGCCAA | miR- | TT+TAA+CAT+GGGGGTA | −0.312 | 8.290 | 40.33 | 3.28 |
| 302c* | 302c_GSP9# | GACAGCAGGTA | 302c-_RP2# | CC | | | | |
| | | SEQ ID NO:333 | | SEQ ID NO:334 | | | | |
| miR-302c | miR- | CATGATCAGCTGGGCCAA | miR- | T+AAG+TGCTTCCATGTT | −0.2945 | 8.381 | 14.28 | 142.76 |
| | 302cGSP9# | GACCACTGAAA | 302CRP5# | TCA | | | | |
| | | SEQ ID NO:335 | | SEQ ID NO:336 | | | | |
| miR-320 | miR- | CATGATCAGCTGGGCCAA | miR- | AAAA+GCT+GGGTTGAGA | −0.2677 | 7.8956 | 6.73 | 67.29 |
| | 320_GSP8# | GATTCGCCCT | 320_RP3# | GG | | | | |
| | | SEQ ID NO:337 | | SEQ ID NO:338 | | | | |
| miR-323 | miR-323GSP | GATGATCAGGTGGGGCAA | miR-323RP | G+CA+CATTACACGGT | −0.2878 | 8.2546 | 0.19 | 1.92 |
| | | GAAGAGGTCGAC | | SEQ ID NO:340 | | | | |
| | | SEQ ID NO:339 | | | | | | |
| miR-324- | miR-324- | GATGATCAGCTGGGCCAA | miR-324- | C+CA+CTGCCCCAGGT | −0.2698 | 8.5223 | 2.54 | 25.41 |
| 3p | 3pGSP | GACCAGCAGCAC | | SEQ ID NO:342 | | | | |
| | | SEQ ID NO:341 | | | | | | |
| miR-324- | miR-324- | CATGATCAGCTGGGCCAA | miR-324- | C+GC+ATCCCGTAGGG | −0.2861 | 7.6865 | 0.06 | 0.62 |
| 5p | 5pGSP | GAACAGCAATGC | | SEQ ID NO:344 | | | | |
| | | SEQ ID NO:343 | | | | | | |
| miR-325 | miR-325GSP | CATGATCAGCTGGGCCAA | miR-325RP | C+CT+AGTAGGTGTCC | −0.2976 | 8.1925 | 0.01 | 0.14 |
| | | GAACACTTACTG | | SEQ ID NO:346 | | | | |
| | | SEQ ID NO:345 | | | | | | |
| miR-326 | miR-326GSP | CATGATCAGCTGGGCCAA | miR-326RP | C+CT+CTGGGGCCCTTC | −0.2806 | 7.897 | 0.59 | 5.87 |
| | | GACTGGAGGAAG | | SEQ ID NO:348 | | | | |
| | | SEQ ID NO:347 | | | | | | |
| miR-328 | miR-328GSP | CATGATCAGCTGGGCCAA | miR-328RP | C+TG+GCCCTCTCTGC | −0.293 | 7.929 | 3.17 | 31.69 |
| | | GAACGGAAGGGC | | SEQ ID NO:350 | | | | |
| | | SEQ ID NO:349 | | | | | | |
| miR-330 | miR-330GSP | CATGATCAGCTGGGCCAAGA | miR-330RP | G+CA+AAGCACACGGC | −0.3009 | 7.7999 | 0.13 | 1.30 |
| | | GTCTCTGCAGG | | SEQ ID NO:352 | | | | |
| | | SEQ ID NO:351 | | | | | | |
| miR-331 | miR-331GSP | CATGATCAGCTGGGCCAA | miR-331RP | G+CC+CCTGGGCCTAT | −0.2816 | 8.1643 | 0.45 | 4.54 |
| | | GATTCTAGGATA | | SEQ ID NO:354 | | | | |
| | | SEQ ID NO:353 | | | | | | |
| miR-337 | miR-337GSP | CATGATCAGCTGGGCCAA | miR-337RP | T+CC+AGCTCCTATATG | −0.2968 | 8.7313 | 0.10 | 1.02 |
| | | GAAAAGGCATCA | | SEQ ID NO:356 | | | | |
| | | SEQ ID NO:355 | | | | | | |
| miR-338 | miR-338GSP | CATGATCAGGTGGGCCAA | miR-338RP2 | T+CC+AGCATCAGTGATT | −0.2768 | 8.5618 | 0.52 | 5.17 |
| | | GATCAACAAAAT | | SEQ ID NO:358 | | | | |
| | | SEQ ID NO:357 | | | | | | |
| miR-339 | miR339GSP9# | CATGATCAGCTGGGCCAG | miR- | T+CC+CTGTCCTCCAGG | −0.303 | 8.4873 | 0.27 | 2.72 |
| | | GATGAGCTCCT | 339RP2# | SEQ ID NO:360 | | | | |
| | | SEQ ID NO:359 | | | | | | |
| miR-340 | miR-340GSP | CATGATCAGCTGGGCCAA | miR-340RP | TC+CG+TCTCAGTTAC | −0.2846 | 9.6673 | 0.15 | 1.45 |
| | | GAGGCTATAAAG | | SEQ ID NO:362 | | | | |
| | | SEQ ID NO:361 | | | | | | |
| miR-342 | miR-342GSP3 | CATGATCAGGTGGGCCAA | miR-342RP | T+CT+CACACAGAAATCG | −0.293 | 8.1553 | 4.69 | 46.85 |
| | | GAGACGGGTG | | SEQ ID NO:364 | | | | |
| | | SEQ ID NO:363 | | | | | | |
| miR-345 | miR-345GSP | CATGATCAGCTGGGCGAA | miR-345RP | T+GC+TGACTCCTAGT | −0.2909 | 8.468 | 0.04 | 0.40 |
| | | GAGCCCTGGACT | | SEQ ID NO:366 | | | | |
| | | SEQ ID NO:365 | | | | | | |
| miR-346 | miR-346GSP | CATGATGAGCTGGGCCAA | miR-346RP | T+GT+CTGCGCGCATG | −0.2959 | 8.1958 | 0.25 | 2.54 |
| | | GAGAGGCAGGC | | SEQ ID NO:368 | | | | |
| | | SEQ ID NO:367 | | | | | | |
| miR-363 | miR-363 | CATGATCAGCTGGGCGAA | miR-363RP# | AAT+TG+CAC+GGTATCC | −0.2362 | 8.9762 | 0.44 | 4.36 |
| | GSP10# | GATACAGATGGA | | SEQ ID NO:370 | | | | |
| | | SEQ ID NO:369 | | | | | | |
| miR-367 | miR-367GSP | CATGATCAGCTGGGCCAA | miR-367RP | AAT+TG+CACTTTAGC | −0.2819 | 8.6711 | 0.00 | 0.03 |
| | | GATCACCATTGC | | AAT | | | | |
| | | SEQ ID NO:371 | | SEQ ID NO:372 | | | | |
| miR-368 | miR-368GSP | CATGATCAGCTGGGCCAA | miR-368RP2 | A+GATAGA+GGAAATT | −0.2953 | 8.0067 | 6.01 | 60.11 |
| | | GAAAACGTGGAA | | CCAC | | | | |
| | | SEQ ID NO:373 | | SEQ ID NO:374 | | | | |
| miR-370 | miR-370GSP | CATGATCAGCTGGGCCAA | miR-370RP | G+CC+TGCTGGGGTGG | −0.2825 | 8.3162 | 1.45 | 14.55 |
| | | GACCAGGTTCCA | | SEQ ID NO:376 | | | | |
| | | SEQ ID NO:375 | | | | | | |
| miR-371 | miR-371GSP | CATGATCAGCTGGGCCAA | miR-371RP | G+TG+CCGCCATCTTT | −0.295 | 7.8812 | 2.51 | 25.12 |
| | | GAACACTCAAAA | | SEQ ID NO:378 | | | | |
| | | SEQ ID NO:377 | | | | | | |
| miR-372 | miR-372GSP | CATGATCAGCTGGGCCAA | miR-372RP | A+AA+GTGCTGCGACA | −0.2984 | 8.9183 | 0.05 | 0.53 |
| | | GAACGCTCAAAT | | SEQ ID NO:380 | | | | |
| | | SEQ ID NO:379 | | | | | | |
| miR-373* | miR-373*GSP | CATGATCAGCTGGGCCAA | miR-373*RP | A+CT+CAAAATGGGGG | −0.2705 | 8.4513 | 0.20 | 1.99 |
| | | GAGGAAAGCGCC | | SEQ ID NO:382 | | | | |
| | | SEQ ID NO:381 | | | | | | |
| miR-373 | miR-373GSP | CATGATCAGCTGGGGCAA | miR-373RP2 | GA+AG+TGCTTCGATTTT | −0.307 | 7.9056 | 9.13 | 91.32 |
| | | GAACACCCCAAA | | G | | | | |
| | | SEQ ID NO:383 | | SEQ ID NO:384 | | | | |
| miR-374 | miR-374GSP2 | CATGATCAGCTGGGCCAA | miR-374RP | TT+AT+AATA+CAACCTG | −0.2655 | 9.3795 | 9.16 | 91.60 |
| | | ACACTTATCA | | ATAAG | | | | |
| | | SEQ ID NO:385 | | SEQ ID NO:386 | | | | |
| miR-375 | miR-375GSP | CATGATCAGCTGGGCCAA | miR-375RP | TT+TG+TTCGTTCGGC | −0.3041 | 8.1181 | 0.09 | 0.90 |
| | | GATCACGCGAGC | | SEQ ID NO:388 | | | | |
| | | SEQ ID NO:387 | | | | | | |
| miR-376b | miR-376b | CATGATCAGCTGGGCCAA | miR- | AT+CAT+AGA+GGAAATC | −0.2934 | 9.0188 | 1.07 | 10.74 |
| | GSP8# | GAAAACATGGA | 376bRP# | CA | | | | |
| | | SEQ ID NO:389 | | SEQ ID NO:390 | | | | |
| miR-378 | miR-378GSP | CATGATCAGGTGGGCCAA | miR-378RP | C+TC+CTGACTCCAGG | −0.2899 | 8.1467 | 0.07 | 0.73 |
| | | GAACACAGGACCC | | SEQ ID NO:392 | | | | |
| | | SEQ ID NO:391 | | | | | | |
| miR-379 | miR- | CATGATCAGCTGGGCCAA | miR- | T+GGT+AGACTATGGAACG | −0.2902 | 8.2149 | 10.89 | 108.86 |
| | 379_GSP7# | GATACGATACGTTC | 379RP2# | AACG | | | | |
| | | SEQ ID NO:393 | | SEQ ID NO:394 | | | | |
| miR-380- | miR-380- | CATGATCAGCTGGGCCAA | miR-380- | T+GGT+TGACCATAGA | −0.2462 | 9.4324 | 1.30 | 13.04 |
| 5p | 5pGSP | GAGCGCATGTTC | 5pRP | SEQ ID NO:396 | | | | |
| | | SEQ ID NO:395 | | | | | | |
| miR-380- | miR-380- | CATGATCAGCTGGGCCAA | miR-380- | TA+TG+TAATATGGTCC | −0.3037 | 8.0356 | 3.69 | 36.89 |
| 3p | 3pGSP | GAAAGATGTGGA | 3pRP | ACA | | | | |
| | | SEQ ID NO:397 | | SEQ ID NO:398 | | | | |
| miR-381 | miR-381GSP2 | CATGATGAGCTGGGCCAA | miR-381RP2 | TATA+CAA+GGGCAAGCT | −0.3064 | 8.8704 | 1.72 | 17.16 |
| | | GAACAGAGAGC | | SEQ ID NO:400 | | | | |
| | | SEQ ID NO:399 | | | | | | |
| miR-382 | miR-382GSP | CATGATCAGCTGGGCCAA | miR-382RP | G+AA+GTTGTTCGTGGT | −0.2803 | 7.6738 | 0.66 | 6.57 |
| | | GACGAATCCACC | | SEQ ID NO:402 | | | | |
| | | SEQ ID NO:401 | | | | | | |
| miR-383 | miR-383GSP | CATGATCAGCTGGGCCAA | miR-383RP2 | A+GATC+AGAAGGTGATT | −0.2866 | 8.1463 | 0.54 | 5.45 |
| | | GAAGCCACAATC | | GT | | | | |
| | | SEQ ID NO:403 | | SEQ ID NO:404 | | | | |
| miR-410 | miR-410 | CATGATCAGCTGGGCCAA | miR-401RP# | AA+TA+TAA+CA+CAGAT | −0.2297 | 8.5166 | 4.27 | 42.71 |
| | GSP9# | GAACAGGCCAT | | GGC | | | | |
| | | SEQ ID NO:405 | | SEQ ID NO:406 | | | | |
| miR-412 | miR-412 | CATGATCAGCTGGGCCAA | miR-412RP# | A+CTT+CACCTGGTCCAC | −0.3001 | 7.9099 | 4.24 | 42.37 |
| | GSP10# | GAACGGCTAGTG | | TA | | | | |
| | | SEQ ID NO:407 | | SEQ ID NO:408 | | | | |
| miR-422a | miR-422aGSP | CATGATCAGCTGGGCCAA | miR-422aRP | C+TG+GACTTAGGGTC | −0.3079 | 9.3108 | 5.95 | 59.54 |
| | | GAGGCCTTCTGA | | SEQ ID NO:410 | | | | |
| | | SEQ ID NO:409 | | | | | | |
| miR-422b | miR-422bGSP | CATGATCAGCTGGGCCAA | miR-422bRP | C+TG+GACTTGGAGTC | −0.2993 | 8.9437 | 4.86 | 48.56 |
| | | GAGGCGTTCTGA | | SEQ ID NO:412 | | | | |
| | | SEQ ID NO:411 | | | | | | |
| miR-423 | miR-423GSP | CATGATCAGCTGGGCCAA | miR-423RP | A+GC+TGGGTCTGAGG | −0.3408 | 9.2274 | 6.06 | 60.62 |
| | | GACTGAGGGGCC | | SEQ ID NO:414 | | | | |
| | | SEQ ID NO:413 | | | | | | |
| miR424 | miR-424GSP# | CATGATCAGCTGGGCCAA | miR- | C+AG+CAGCAATTCATGT | −0.3569 | 9.3419 | 10.78 | 107.85 |
| | | GATTCAAAACAT | 424RP2# | TTT | | | | |
| | | SEQ ID NO:415 | | SEQ ID NO:416 | | | | |
| miR-425 | miR-425GSP | CATGATCAGCTGGGCCAA | miR-425RP | A+TC+GGGAATGTCGT | −0.2932 | 7.9786 | 0.39 | 3.93 |
| | | GAGGCGGACACG | | SEQ ID NO:418 | | | | |
| | | SEQ ID NO:417 | | | | | | |
| miR-429 | miR- | CATGATCAGCTGGGCCAA | miR- | T+AATAC+TG+TCTGGTA | −0.2458 | 8.2805 | 16.21 | 162.12 |
| | 429_GSP11# | GAACGGTTTTACC | 429RP5# | AAA | | | | |
| | | SEQ ID NO:419 | | SEQ ID NO:420 | | | | |
| miR-431 | miR-431 | CATGATCAGCTGGGCCAA | miR-431RP# | T+GT+CTTGCAGGCCG | −0.3107 | 7.7127 | 7.00 | 70.05 |
| | GSP10# | GATGCATGACGG | | SEQ ID NO:422 | | | | |
| | | SEQ ID NO:421 | | | | | | |
| miR-448 | miR-448GSP | CATGATCAGCTGGGCCAA | miR-448RP | TTG+CATA+TGTAGGATG | −0.3001 | 8.4969 | 0.12 | 1.16 |
| | | GAATGGGACATC | | SEQ ID NO:424 | | | | |
| | | SEQ ID NO:423 | | | | | | |
| miR-449 | miR- | CATGATCAGCTGGGCCAA | miR- | T+GG+CAGTGTATTGTTT | −0.3225 | 8.4953 | 2.57 | 25.70 |
| | 449GSP10# | GAACCAGCTAAC | 449RP2# | AGC | | | | |
| | | SEQ ID NO:425 | | SEQ ID NO:426 | | | | |
| miR-450 | miR-450GSP | CATGATCAGCTGGGCCAA | miR-450RP | TTTT+TG+GGATGTGTT | −0.2906 | 8.1404 | 0.48 | 4.82 |
| | | GATATTAGGAAC | | SEQ ID NO:428 | | | | |
| | | SEQ ID NO:427 | | | | | | |
| miR-451 | miR-451 | CATGATCAGCTGGGCCAA | miR-451RP# | AAA+CCG+TTA+CCATTA | −0.2544 | 8.0291 | 1.73 | 17.35 |
| | GSP10# | GAAAACTCAGTA | | CTGA | | | | |
| | | SEQ ID NO:429 | | SEQ ID NO:430 | | | | |
| let7a | let7a-GSP2# | CATGATCAGCTGGGCCAA | let7a-RP# | T+GA+GGTAGTAGGTTG | −0.3089 | 9.458 | 0.04 | 0.38 |
| | | GAAACTATAC | | SEQ ID NO:432 | | | | |
| | | SEQ ID NO:431 | | | | | | |
| let7b | let7b-GSP2# | CATGATCAGCTGGGCCAA | let7b-RP# | T+GA+GGTAGTAGGTTG | −0.2978 | 7.9144 | 0.05 | 0.54 |
| | | GAAACGACAC | | SEQ ID NO:432 | | | | |
| | | SEQ ID NO:433 | | | | | | |
| let7c | let7c-GSP211 | CATGATCAGCTGGGCCAA | let7c-RP11 | T+GA+GGTAGTAGGTTG | −0.308 | 7.9854 | 0.01 | 0.14 |
| | | GAAACCATAC | | SEQ ID NO:432 | | | | |
| | | SEQ ID NO:434 | | | | | | |
| let7d | let7d-GSP2# | CATGATCAGCTGGGCCAA | Iet7d-RP# | A+GA+GGTAGTAGGTTG | −0.3238 | 8.3359 | 0.06 | 0.57 |
| | | GAACTATGCA | | SEQ ID NO:436 | | | | |
| | | SEQ ID NO:435 | | | | | | |
| let7e | let7e-GSP2# | CATGATCAGCTGGGCCAA | let7e-RP# | T+GA+GGTAGGAGGTTG | −0.3284 | 9.7594 | 0.22 | 2.20 |
| | | GAACTATACA | | SEQ ID NO:438 | | | | |
| | | SEQ ID NO:437 | | | | | | |
| let7f | 1et7f-GSP2# | CATGATCAGCTGGGCCAA | let7f-RP# | T+GA+GGTAGTAGATTG | −0.2901 | 11.107 | 0.32 | 3.18 |
| | | GAAACTATAC | | SEQ ID NO:440 | | | | |
| | | SEQ ID NO:439 | | | | | | |
| let7g | let7g-GSP2# | CATGATCAGCTGGGCCAA | let7g-RP# | T+GA+GGTAGTAGTTTG | −0.3469 | 9.8235 | 0.16 | 1.64 |
| | | GAACTGTACA | | SEQ ID NO:442 | | | | |
| | | SEQ ID NO:441 | | | | | | |
| let7i | let7i-GSP2# | CATGATCAGCTGGGCCAA | let7i-RP# | T+GA+GGTAGTAGTTTG | −0.321 | 10.82 | 0.20 | 1.99 |
| | | GAACAGCACA | | SEQ ID NO:444 | | | | |
| | | SEQ ID NO:443 | | | | | | |
| miR-377 | miR-377GSP | CATGATCAGCTGGGCCAA | miR-377RP2 | AT+CA+CACAAAGGCAAC | −0.2979 | 10.612 | 13.45 | 134.48 |
| | | GAACAAAAGTTG | | SEQ ID NO:446 | | | | |
| | | SEQ ID NO:445 | | | | | | |
| miR-376a | miR- | CATGATGAGCTGGGCCAA | miR- | AT+CAT+AGA+GGAAAAT | −0.2938 | 10.045 | 63.00 | 630.00 |
| | 376a_GSP7 | GAACGTGGA | 376a_RP5 | CC | | | | |
| | | SEQ ID NO:447 | | SEQ ID NO:448 | | | | |
| miR-22 | miR-22GSP | CATGATCAGCTGGGCCAA | miR-22RP | A+AG+CTGCCAGTTGA | −0.2862 | 8.883 | 20.46 | 204.58 |
| | | GAACAGTTCTTC | | SEQ ID NO:450 | | | | |
| | | SEQ ID NO:449 | | | | | | |
| miR-200c | miR-200cGSP2 | CATGATCAGCTGGGCCAA | miR-200cRP | TAA+TACTGCCGGGT | −0.3094 | 11.5 | 15.99 | 159.91 |
| | | GACCATCATTA | | SEQ ID NO:452 | | | | |
| | | SEQ ID NO:451 | | | | | | |
| miR-24 | miR-24GSP | CATGATCAGCTGGGCCAA | miR-24RP | T+GG+CTCAGTTCAGC | −0.3123 | 8.6824 | 24.34 | 243.38 |
| | | GACTGTTCCTGC | | SEQ ID NO:454 | | | | |
| | | SEQ ID NO:453 | | | | | | |
| miR- | miR-29cGSP10 | CATGATCAGCTGGGCCAA | miR-29cRP | T+AG+CACCATTGAAAT | −0.2975 | 8.8441 | 23.22 | 232.17 |
| 29cDNA | | GAACCGATTCA | | SEQ ID NO:456 | | | | |
| | | SEQ ID NO:455 | | | | | | |
| miR-18 | miR-18GSP | CATGATCAGCTGGGCCAA | miR-18RP | T+AA+GGTGCATCTAGT | −0.3209 | 9.0999 | 14.90 | 149.01 |
| | | GATATCTGCACT | | SEQ ID NO:458 | | | | |
| | | SEQ ID NO:457 | | | | | | |
| miR-185 | miR-185GSP | CATGATCAGCTGGGCCAA | miR-185RP | T+GG+AGAGAAAGGCA | −0.3081 | 8.9289 | 15.73 | 157.32 |
| | | GAGAACTGCCTT | | SEQ ID NO:460 | | | | |
| | | SEQ ID NO:459 | | | | | | |
| miR-181b | miR- | CATGATCAGCTGGGCCAA | miR- | AA+CATT+CATTGCTGTC | −0.3115 | 10.846 | 15.87 | 158.67 |
| | 181bGSP8# | GACCCACCGA | 181bRP2# | SEQ ID NO:462 | | | | |
| | | SEQ ID NO:461 | | | | | | |
| miR-128a | miR-128aGSP | CATGATGAGCTGGGCCAA | miR- | TCAGAGTGAACCGGT | approx. | approx. | approx. | approx. |
| | | GAAAAAGAGACC | 128-anLRP | SEQ ID NO: 494 | −0.2866 | 8.0867 | 0.16 | 1.60 |
| | | SEQ ID NO:161 | | | | | | |
| miR-138 | miR-138GSP2 | CATGATCAGCTGGGCCAA | miR- | AGCTGGTGTTGTGAA | approx. | approx. | approx. | approx. |
| | | GACGGCGTGAT | 138nLRP | SEQ ID NO:495 | −0.3023 | 9.0814 | 0.22 | 2.19 |
| | | SEQ ID NO:187 | | | | | | |
| miR-143 | miR-143GSP8- | CATGATCAGCTGGGCCAA | miR- | TGAGATGAAGCACTGT | approx. | approx. | approx. | approx. |
| | | GATGAGCTAC | 143nLRP | SEQ ID NO:496 | −0.3008 | 9.2675 | 0.37 | 3.71 |
| | | SEQ ID NO:197 | | | | | | |
| miR-150 | miR-150GSP3 | CATGATCAGCTGGGCCAA | miR- | TCTCCCAACCCTTGTA | approx. | approx. | approx. | approx. |
| | | GACACTGGTA | 150nLRP | SEQ ID NO:497 | −0.2943 | 8.3945 | 0.06 | 0.56 |
| | | SEQ ID NO:213 | | | | | | |
| miR-181a | miR- | CATGATCAGCTGGGCCAA | miR- | AACATTCAACGCTGT | approx. | approx. | approx. | approx. |
| | 181aGSP9# | GAAGTCACCGA | 181anLRP | SEQ ID NO: 498 | −0.2919 | 7.968 | 1.70 | 17.05 |
| | | SEQ ID NO:227 | | | | | | |
| miR-194 | mir194GSP8# | CATGATGAGCTGGGGCAA | miR- | TGTAACAGCAACTCCA | approx. | approx. | approx. | approx. |
| | | GATCCACATG | 194nLRP | SEQ ID NO: 499 | −0.3078 | 8.8045 | 0.37 | 3.69 |
| | | SEQ ID NO:255 | | | | | | |

# denotes primers for assays that required extensive testmg and primer design modification to achieve optimal assay results mcludmg high sensitivity and high dynamic range.

### EXAMPLE 4

This Example describes assays and primers designed for quantitative analysis of murine miNRA expression patterns.

Methods: The representative murine microRNA target templates described in TABLE 7 are publicly available accessible on the World Wide Web at the Wellcome Trust Sanger Institute website in the “miRBase sequence database” as described in Griffith-Jones et al. (2004), Nucleic Acids Research 32:D109-D111 and Griffith-Jones et al. (2006), Nucleic Acids Research 34: D140-D144. As indicated below in TABLE 7, the murine microRNA templates are either totally identical to the corresponding human microRNA templates, identical in the overlapping sequence with differing ends, or contain one or more base pair changes as compared to the human microRNA sequence. The murine microRNA templates that are identical or that have identical overlapping sequence to the corresponding human templates can be assayed using the same primer sets designed for the human microRNA templates, as indicated in TABLE 7. For the murine microRNA templates with one or more base pair changes in comparison to the corresponding human templates, primer sets have been designed specifically for detection of the murine microRNA, and these primers are provided in TABLE 7. The extension primer reaction and quantitative PCR reactions for detection of the murine microRNA templates may be carried out as described in EXAMPLE 3.

TABLE 7
Primers to detect murine microRNA target templates

| Mouse | Exten- | | Reverse | | Mouse microRNA |
|---|---|---|---|---|---|
| Target | sion Primer | Extension | Primer | Reverse | as compared to |
| microRNA: | Name | Primer Sequence | Name | Primer Sequence | Human microRNA |
| miR-1 | miR1GSP10 | CATGATCAGCTGGGCCAAGATACATA | miR-1RP | T+G+GAA+TG+TAAAGAAGT | Identical |
| | | CTTC | | SEQ ID NO:48 | |
| | | SEQ ID NO:47 | | | |
| miR-7 | miR-7GSP10 | CATGATCAGCTGGGCCAAGAAACAAA | miR-7_RP6 | T+GGAA+GACTTGTGATTTT | one or more base |
| | | ATC | | SEQ ID NO:487 | pairs differ |
| | | SEQ ID NO:486 | | | |
| miR-9* | miR-9*GSP | CATGATCAGCTGGGCCAAGAACTTTC | miR-9*RP | TAAA+GCT+AGATAACCG | Identical overlapping |
| | | GGTT | | SEQ ID NO:52 | sequence, ends differ |
| | | SEQ ID NO:51 | | | |
| miR-10a | miR-10aGSP | CATGATCAGCTGGGCCAAGACACAAA | miR-10aRP | T+AC+CCTGTAGATCCG | Identical |
| | | TTCG | | SEQ ID NO:54 | |
| | | SEQ ID NO:53 | | | |
| miR-10b | miR-10b_GSP11 | CATGATCAGCTGGGCCAAGAACACAA | miR-10b_RP2 | C+CC+TGT+AGAACCGAAT | one or more base |
| | | ATTCG | | SEQ ID NO:493 | pairs differ |
| | | SEQ ID NO:492 | | | |
| miR-15a | miR-15aGSP | CATGATCAGCTGGGCCAAGACACAAA | miR-15aRP | T+AG+CAGCACATAATG | Identical |
| | | CCAT | | SEQ ID NO:58 | |
| | | SEQ ID NO:57 | | | |
| miR-15b | miR-15bGSP2 | CATGATCAGCTGGGCCAAGATGTAAA | miR-15bRP | T+AG+CAGCACATCAT | Identical |
| | | CCA | | SEQ ID NO:60 | |
| | | SEQ ID NO:59 | | | |
| miR-16 | miR-16GSP2 | CATGATCAGCTGGGCCAAGACGCCAA | miR-16RP | T+AG+CAGCACGTAAA | Identical |
| | | TAT | | SEQ ID NO:62 | |
| | | SEQ ID NO:61 | | | |
| miR-17-3p | miR-17-3pGSP | CATGATCAGCTGGGCCAAGAACAAGT | miR-17-3pRP | A+CT+GCAGTGAGGGC | one or more base |
| | | GCCC | | SEQ ID NO:464 | pairs differ |
| | | SEQ ID NO:463 | | | |
| miR-17-5p | miR-17-5pGSP2 | CATGATCAGCTGGGCCAAGAACTACC | miR-17-5pRP | C+AA+AGTGCTTACAGTG | Identical |
| | | TGC | | SEQ ID NO:66 | |
| | | SEQ ID NO:65 | | | |
| miR-19a | miR-19aGSP2 | CATGATCAGCTGGGCCAAGATCAGTT | miR-19aRP | TG+TG+CAAATCTATGC | Identical |
| | | TTG | | SEQ ID NO:68 | |
| | | SEQ ID NO:67 | | | |
| miR-19b | miR-19bGSP | CATGATCAGCTGGGCCAAGATCAGTT | miR-19bRP | TG+TG+CAAATCCATG | Identical |
| | | TTGC | | SEQ ID NO:70 | |
| | | SEQ ID NO:69 | | | |
| miR-20 | miR-20GSP3 | CATGATCAGCTGGGCCAAGACTACCT | miR-20RP | T+AA+AGTGCTTATAGTGCA | Identical |
| | | GC | | SEQ ID NO:72 | |
| | | SEQ ID NO:71 | | | |
| miR-21 | miR-21GSP2 | CATGATCAGCTGGGCCAAGATCAACA | miR-21RP | T+AG+CTTATCAGACTGATG | Identical |
| | | TCA | | SEQ ID NO:74 | |
| | | SEQ ID NO:73 | | | |
| miR-23a | miR-23aGSP | CATGATCAGCTGGGCCAAGAGGAAAT | miR-23aRP | A+TC+ACATTGCCAGG | Identical |
| | | CCCT | | SEQ ID NO:76 | |
| | | SEQ ID NO:75 | | | |
| miR-23b | miR-23bGSP | CATGATCAGCTGGGCCAAGAGGTAAT | miR-23bRP | A+TC+ACATTGCCAGG | Identical |
| | | CCCT | | SEQ ID NO:78 | |
| | | SEQ ID NO:77 | | | |
| miR-24 | miR-24P5 | CATGATCAGCTGGGCCAAGACTGTTC | miR24-1, 2R | TGG+CTCAGTTCAGC | Identical |
| | | CTGCTG | | SEQ ID NO: 19 | |
| | | SEQ ID NO:7 | | | |
| miR-25 | miR-25GSP | CATGATCAGCTGGGCCAAGATCAGAC | miR-25RP | C+AT+TGCACTTGTCTC | Identical |
| | | CGAG | | SEQ ID NO:80 | |
| | | SEQ ID NO:79 | | | |
| miR-26a | miR-26aGSP9 | CATGATCAGCTGGGCCAAGAGCCTAT | miR-26aRP2 | TT+CA+AGTAATCCAGGAT | Identical |
| | | CCT | | SEQ ID NO:82 | |
| | | SEQ ID NO:81 | | | |
| miR-26b | miR-26bGSP9 | CATGATCAGCTGGGCCAAGAAACCTA | miR-26bRP2 | TT+CA+AGT+AATTCAGGAT | Identical |
| | | TCC | | SEQ ID NO:84 | |
| | | SEQ ID NO:83 | | | |
| miR-27a | miR-27aGSP | CATGATCAGCTGGGCCAAGAGCGGAA | miR-27aRP | TT+CA+CAGTGGCTAA | Identical |
| | | CTTA | | SEQ ID NO:86 | |
| | | SEQ ID NO:85 | | | |
| miR-27b | miR-27bGSP | CATGATCAGCTGGGCCAAGAGCAGAA | miR-27bRP | TT+CA+CAGTGGCTAA | Identical |
| | | CTTA | | SEQ ID NO:88 | |
| | | SEQ ID NO:87 | | | |
| miR-28 | miR-28GSP | CATGATCAGCTGGGCCAAGACTCAAT | miR-28RP | A+AG+GAGCTCACAGT | Identical |
| | | AGAC | | SEQ ID NO:90 | |
| | | SEQ ID NO:89 | | | |
| miR-29a | miR-29aGSP8 | CATGATCAGCTGGGCCAAGAAACCGA | miR-29aRP2 | T+AG+CACCATCTGAAAT | Identical |
| | | TT | | SEQ ID NO:92 | |
| | | SEQ ID NO:91 | | | |
| miR-29b | miR-29bGSP2 | CATGATCAGCTGGGCCAAGAAACACT | miR-29bRP2 | T+AG+CACCATTTGAAATCAG | Identical |
| | | GAT | | SEQ ID NO:94 | |
| | | SEQ ID NO:93 | | | |
| miR-30a- | miR-30a-5pGSP | CATGATCAGCTGGGCCAAGACTTCCA | miR30a-5pRP | T+GT+AAACATCCTCGAC | Identical |
| 5p | | GTCG | | SEQ ID NO:96 | |
| | | SEQ ID NO:95 | | | |
| miR-30b | miR-30bGSP | CATGATCAGCTGGGCCAAGAAGCTGA | miR-30bRP | TGT+AAA+CATCCTACACT | Identical |
| | | GTGT | | SEQ ID NO:98 | |
| | | SEQ ID NO:97 | | | |
| miR-30c | miR-30cGSP | CATGATCAGCTGGGCCAAGAGCTGAG | miR-30cRP | TGT+AAA+CATCCTACACT | Identical |
| | | AGTG | | SEQ ID NO:100 | |
| | | SEQ ID NO:99 | | | |
| miR-30d | miR-30dGSP | CATGATCAGCTGGGCCAAGACTTCCA | miR-30dRP | T+GTAAA+CATCCCCG | Identical |
| | | GTCG | | SEQ ID NO:102 | |
| | | SEQ ID NO:101 | | | |
| miR-30e- | miR-30e- | CATGATCAGCTGGGCCAAGAGCTGTA | miR-30e- | CTTT+CAGT+CGGATGTTT | Identical |
| 3p | 3pGSP9 | AAC | 3pRP5 | SEQ ID NO:104 | |
| | | SEQ ID NO:103 | | | |
| miR-31 | miR-31GSP | CATGATCAGCTGGGCCAAGACAGCTA | miR-31RP | G+GC+AAGATGCTGGC | Identical overlapping |
| | | TGCC | | SEQ ID NO:108 | sequence, ends differ |
| | | SEQ ID NO:107 | | | |
| miR-32 | miR-32GSP | CATGATCAGCTGGGCCAAGAGCAACT | miR-32RP | TATTG+CA+CATTACTAAG | Identical |
| | | TAGT | | SEQ ID NO:110 | |
| | | SEQ ID NO:109 | | | |
| miR-33 | miR-33GSP2 | CATGATCAGCTGGGCCAAGACAATGC | miR-33RP | G+TG+CATTGTAGTTGC | Identical |
| | | AAC | | SEQ ID NO:112 | |
| | | SEQ ID NO:111 | | | |
| miR-34a | miR-34aGSP | CATGATCAGCTGGGCCAAGAAACAAC | miR-34aRP | T+GG+CAGTGTCTTAG | Identical |
| | | CAGC | | SEQ ID NO:114 | |
| | | SEQ ID NO:113 | | | |
| miR-34b | miR-34bGSP | CATGATCAGCTGGGCCAAGACAATCA | miR-34bRP | TA+GG+CAGTGTAATT | one or more base |
| | | GCTA | | SEQ ID NO:482 | pairs differ |
| | | SEQ ID NO:115 | | | |
| miR-34c | miR-34cGSP | CATGATCAGCTGGGCCAAGAGCAATC | miR-34cRP | A+GG+CAGTGTAGTTA | Identical |
| | | AGCT | | SEQ ID NO:118 | |
| | | SEQ ID NO:117 | | | |
| miR-92 | miR-92GSP | CATGATCAGCTGGGCCAAGACAGGCC | miR-92RP | T+AT+TGCACTTGTCCC | Identical |
| | | GGGA | | SEQ ID NO:120 | |
| | | SEQ ID NO:119 | | | |
| miR-93 | miR-93GSP | CATGATCAGCTGGGCCAAGACTACCT | miR93RP | AA+AG+TGCTGTTCGT | Identical overlapping |
| | | GCAC | | SEQ ID NO:122 | sequence, ends differ |
| | | SEQ ID NO:121 | | | |
| miR-96 | miR-96GSP | CATGATCAGCTGGGCCAAGAGCAAAA | miR96RP | T+TT+GGCACTAGCAC | Identical overlapping |
| | | ATGT | | SEQ ID NO:126 | sequence, ends differ |
| | | SEQ ID NO:125 | | | |
| miR-98 | miR-98GSP | CATGATCAGCTGGGCCAAGAAACAAT | miR-98RP | TGA+GGT+AGTAAGTTG | Identical |
| | | ACAA | | SEQ ID NO:128 | |
| | | SEQ ID NO:127 | | | |
| miR-99a | miR-99aGSP | CATGATCAGCTGGGCCAAGACACAAG | miR-99aRP | A+AC+CCGTAGATCCG | Identical overlapping |
| | | ATCG | | SEQ ID NO:130 | sequence, ends differ |
| | | SEQ ID NO:129 | | | |
| miR-99b | miR-99bGSP | CATGATCAGCTGGGCCAAGACGCAAG | miR-99bRP | C+AC+CCGTAGAACCG | Identical |
| | | GTCG | | SEQ ID NO:132 | |
| | | SEQ ID NO:131 | | | |
| miR-100 | miR-100GSP | CATGATCAGCTGGGCCAAGACACAAG | miR-100RP | A+AC+CCGTAGATCCG | Identical |
| | | TTCG | | SEQ ID NO:134 | |
| | | SEQ ID NO:133 | | | |
| miR-101 | miR-101GSP | CATGATCAGCTGGGCCAAGACTTCAG | miR-101RP | TA+CAG+TACTGTGATAACT | Identical |
| | | TTAT | | SEQ ID NO:136 | |
| | | SEQ ID NO:135 | | | |
| miR-103 | miR-103GSP | CATGATCAGCTGGGCCAAGATCATAG | miR-103RP | A+GC+AGCATTGTACA | Identical |
| | | CCCT | | SEQ ID NO:138 | |
| | | SEQ ID NO:137 | | | |
| miR-106a | miR-106aGSP | CATGATCAGCTGGGCCAAGATACCTG | miR-106aRP | CAA+AG+TGCTAACAGTG | one or more base |
| | | CAC | | SEQ ID NO:473 | pairs differ |
| | | SEQ ID NO:472 | | | |
| miR-106b | miR-106bGSP | CATGATCAGCTGGGCCAAGAATCTGC | miR-106bRP | T+AAAG+TGCTGACAGT | Identical |
| | | ACTG | | SEQ ID NO:144 | |
| | | SEQ ID NO:143 | | | |
| miR-107 | miR-107GSP8 | CATGATCAGCTGGGCCAAGATGATAG | miR-107RP2 | A+GC+AGCATTGTACAG | Identical |
| | | CC | | SEQ ID NO:146 | |
| | | SEQ ID NO:145 | | | |
| miR-122a | miR-122aGSP | CATGATCAGCTGGGCCAAGAACAAAC | miR-122aRP | T+GG+AGTGTGACAAT | Identical |
| | | ACCA | | SEQ ID NO:148 | |
| | | SEQ ID NO:147 | | | |
| miR-124a | miR-124aGSP | CATGATCAGCTGGGCCAAGATGGCAT | miR-124aRP | T+TA+AGGCACGCGGT | Identical overlapping |
| | | TCAC | | SEQ ID NO:150 | sequence, ends differ |
| | | SEQ ID NO:149 | | | |
| miR-125a | miR-125aGSP | CATGATCAGCTGGGCCAAGACACAGG | miR-125aRP | T+CC+CTGAGACCCTT | Identical |
| | | TTAA | | SEQ ID NO:152 | |
| | | SEQ ID NO:151 | | | |
| miR-125b | miR-125bGSP | CATGATCAGCTGGGCCAAGATCACAA | miR-125bRP | T+CC+CTCAGACCCTA | Identical |
| | | GTTA | | SEQ ID NO:154 | |
| | | SEQ ID NO:153 | | | |
| miR-126 | miR-126GSP | CATGATCAGCTGGGCCAAGAGCATTA | miR-126R2 | T+CG+TACCGTGAGTA | Identical |
| | | TTAC | | SEQ ID NO:156 | |
| | | SEQ ID NO:155 | | | |
| miR-126* | miR-126*GSP3 | CATGATCAGCTGGGCCAAGACGCGTA | miR-126*RP | C+ATT+ATTA+CTTTTGGT | Identical |
| | | CC | | ACG | |
| | | SEQ ID NO:157 | | SEQ ID NO:158 | |
| miR-127 | miR-127GSP | CATGATCAGCTGGGCCAAGAAGCCAA | miR-127RP | T+CG+GATCCGTCTGA | Identical overlapping |
| | | GCTC | | SEQ ID NO:160 | sequence, ends differ |
| | | SEQ ID NO:159 | | | |
| miR-128a | miR-128aGSP | CATGATCAGCTGGGCCAAGAAAAAGA | miR-128aRP | T+CA+CAGTGAACCGG | Identical |
| | | GACC | | SEQ ID NO:162 | |
| | | SEQ ID NO:161 | | | |
| miR-128b | miR-128bGSP | CATGATCAGCTGGGCCAAGAGAAAGA | miR-128bRP | T+CA+CAGTGAACCGG | Identical |
| | | GACC | | SEQ ID NO:164 | |
| | | SEQ ID NO:163 | | | |
| miR-130a | miR-130aGSP | CATGATCAGCTGGGCCAAGAATGCCC | miR-130aRP | C+AG+TGCAATGTTAAAAG | Identical |
| | | TTTT | | SEQ ID NO:168 | |
| | | SEQ ID NO:167 | | | |
| miR-130b | miR-130bGSP | CATGATCAGCTGGGCCAAGAATGCCC | miR-130bRP | C+AG+TGCAATGATGA | Identical |
| | | TTTC | | SEQ ID NO:170 | |
| | | SEQ ID NO:169 | | | |
| miR-132 | miR-132GSP | CATGATCAGCTGGGCCAAGACGACCA | miR-132RP | T+AA+CAGTCTACAGCC | Identical |
| | | TGGC | | SEQ ID NO:172 | |
| | | SEQ ID NO:171 | | | |
| miR-133a | miR-133aGSP | CATGATCAGCTGGGCCAAGAACAGCT | miR-133aRP | T+TG+GTCCCCTTCAA | Identical |
| | | GGTT | | SEQ ID NO:174 | |
| | | SEQ ID NO:173 | | | |
| miR-133b | miR-133bGSP | CATGATCAGCTGGGCCAAGATAGCTG | miR-133bRP | T+TG+GTCCCCTTCAA | Identical |
| | | GTTG | | SEQ ID NO:176 | |
| | | SEQ ID NO:175 | | | |
| miR-134 | miR-134GSP | CATGATCAGCTGGGCCAAGACCCTCT | miR-134RP | T+GT+GACTGGTTGAC | Identical overlapping |
| | | GGTC | | SEQ ID NO:178 | sequence, ends differ |
| | | SEQ ID NO:177 | | | |
| miR-135a | miR-135aGSP | CATGATCAGCTGGGCCAAGATCACAT | miR-135aRP | T+AT+GGCTTTTTATTCCT | Identical |
| | | AGGA | | SEQ ID NO:180 | |
| | | SEQ ID NO:179 | | | |
| miR-135b | miR-135bGSP | CATGATCAGCTGGGCCAAGACACATA | miR-135bRP | T+AT+GGCTTTTCATTCC | Identical |
| | | GGAA | | SEQ ID NO:182 | |
| | | SEQ ID NO:181 | | | |
| miR-136 | miR-136GSP | CATGATCAGCTGGGCCAAGATCCATC | miR-136RP | A+CT+CCATTTGTTTTGATG | Identical |
| | | ATCA | | SEQ ID NO:184 | |
| | | SEQ ID NO:183 | | | |
| miR-137 | miR-137GSP | CATGATCAGCTGGGCCAAGACTACGC | miR-137RP | T+AT+TGCTTAAGAATACGC | Identical overlapping |
| | | GTAT | | SEQ ID NO:186 | sequence, ends differ |
| | | SEQ ID NO:185 | | | |
| miR-138 | miR-138GSP2 | CATGATCAGCTGGGCCAAGACGGCCT | miR-138RP | A+GC+TGGTGTTGTGA | Identical |
| | | GAT | | SEQ ID NO:188 | |
| | | SEQ ID NO:187 | | | |
| miR-139 | miR-139GSP | CATGATCAGCTGGGCCAAGAAGACAC | miR-139RP | T+CT+ACAGTGCACGT | Identical |
| | | GTGC | | SEQ ID NO:190 | |
| | | SEQ ID NO:189 | | | |
| miR-140 | miR-140GSP | CATGATCAGCTGGGCCAAGACTACCA | miR-140RP | A+GT+GGTTTTACCCT | Identical overlapping |
| | | TAGG | | SEQ ID NO:192 | sequence, ends differ |
| | | SEQ ID NO:191 | | | |
| miR-141 | miR-141GSP9 | CATGATCAGCTGGGCCAAGACCATCT | miR-141RP2 | TAA+CAC+TGTCTGGTAA | Identical |
| | | TTA | | SEQ ID NO:194 | |
| | | SEQ ID NO:193 | | | |
| miR-142- | miR-142- | CATGATCAGCTGGGCCAAGATCCATA | miR-142- | TGT+AG+TGTTTCCTACT | Identical overlapping |
| 3p | 3pGSP3 | AA | 3pRP | SEQ ID NO:196 | sequence, ends differ |
| | | SEQ ID NO:195 | | | |
| miR-143 | miR-143GSP8 | CATGATCAGCTGGGCCAAGATGAGCT | miR-143RP2 | T+GA+GATGAAGCACTG | Identical |
| | | AC | | SEQ ID NO:198 | |
| | | SEQ ID NO:197 | | | |
| miR-144 | miR-144GSP2 | CATGATCAGCTGGGCCAAGACTAGTA | miR-144RP | TA+CA+GTAT+AGATGATG | Identical |
| | | CAT | | SEQ ID NO:200 | |
| | | SEQ ID NO:199 | | | |
| miR-145 | miR-145GSP2 | CATGATCAGCTGGGCCAAGAAAGGGA | miR-145RP | G+TC+CAGTTTTCCCA | Identical |
| | | TTC | | SEQ ID NO:202 | |
| | | SEQ ID NO:201 | | | |
| miR-146 | miR-146GSP3 | CATGATCAGCTGGGCCAAGAAACCCA | miR-146RP | T+GA+GAACTGAATTCCA | Identical |
| | | TG | | SEQ ID NO:204 | |
| | | SEQ ID NO:203 | | | |
| miR-148a | miR-148aGSP2 | CATGATCAGCTGGGCCAAGAACAAAG | miR-148aRP2 | T+CA+GTGCACTACAGAACT | Identical |
| | | TTC | | SEQ ID NO:208 | |
| | | SEQ ID NO:207 | | | |
| miR-148b | miR-148bGSP2 | CATGATCAGCTGGGCCAAGAACAAAG | miR-148bRP | T+CA+GTGCATCACAG | Identical |
| | | TTC | | SEQ ID NO:210 | |
| | | SEQ ID NO:209 | | | |
| miR-149 | miR-149GSP2 | CATGATCAGCTGGGCCAAGAGGAGTG | miR-149RP | T+CT+GGCTCCGTGTC | Identical |
| | | AAG | | SEQ ID NO:212 | |
| | | SEQ ID NO:211 | | | |
| miR-150 | miR-150GSP3 | CATGATCAGCTGGGCCAAGACACTGG | miR-150RP | T+CT+CCCAACCCTTG | Identical |
| | | TA | | SEQ ID NO:214 | |
| | | SEQ ID NO:213 | | | |
| miR-151 | miR-151GSP2 | CATGATCAGCTGGGCCAAGACCTCAA | miR-151RP | A+CT+AGACTGAGGCTC | one or more base |
| | | GGA | | SEQ ID NO:477 | pairs differ |
| | | SEQ ID NO: 215 | | | |
| miR-152 | miR-152GSP2 | CATGATCAGCTGGGCCAAGACCCAAG | miR-152RP | T+CA+GTGCATGACAG | Identical |
| | | TTC | | SEQ ID NO:218 | |
| | | SEQ ID NO:217 | | | |
| miR-153 | miR-153GSP2 | CATGATCAGCTGGGCCAAGATCACTT | miR-153RP | TTG+CAT+AGTCACAAAA | Identical overlapping |
| | | TTG | | SEQ ID NO:220 | sequence, ends differ |
| | | SEQ ID NO:219 | | | |
| miR-154 | miR-154GSP9 | CATGATCAGCTGGGCCAAGACGAAGG | miR-154RP3 | TA+GGTTA+TCCGTGTT | Identical |
| | | CAA | | SEQ ID NO:224 | |
| | | SEQ ID NO:223 | | | |
| miR-155 | miR-155GSP8 | CATGATCAGCTGGGCCAAGACCCCTA | miR-155RP2 | TT+AA+TGCTAATTGTGATA | one or more base |
| | | TC | | GG | pairs differ |
| | | SEQ ID NO:225 | | SEQ ID NO:489 | |
| miR-181a | miR- | CATGATCAGCTGGGCCAAGAACTCAC | miR-181aRP2 | AA+CATT+CAACGCTGTC | Identical |
| | 181aGSP9 | CGA | | SEQ ID NO:228 | |
| | | SEQ ID NO:227 | | | |
| miR-181c | miR- | CATGATCAGCTGGGCCAAGAACTCAC | miR-181cRP2 | AA+CATT+CAACCTGTCG | Identical |
| | 181cGSP9 | CGA | | SEQ ID NO:230 | |
| | | SEQ. ID NO:229 | | | |
| miR-182 | miR-182*GSP | CATGATCAGCTGGGCCAAGATAGTTG | miR-182*RP | T+GG+TTCTAGACTTGC | Identical |
| | | GCAA | | SEQ ID NO:232 | |
| | | SEQ ID NO:231 | | | |
| miR-183 | miR-183GSP2 | CATGATCAGCTGGGCCAAGACAGTGA | miR-183RP | T+AT+GGCACTGGTAG | Identical |
| | | ATT | | SEQ ID NO:236 | |
| | | SEQ ID NO:235 | | | |
| miR-184 | miR-184GSP2 | CATGATCAGCTGGGCCAAGAACCCTT | miR-184RP | T+GG+ACGGAGAACTG | Identical |
| | | ATC | | SEQ ID NO:238 | |
| | | SEQ ID NO:237 | | | |
| miR-186 | miR-186GSP9 | CATGATCAGCTGGGCCAAGAAAGCCC | miR-186RP3 | CA+AA+GAATT+CTCCTTTT | Identical |
| | | AAA | | GG | |
| | | SEQ ID NO:239 | | SEQ ID NO:240 | |
| miR-187 | miR-187GSP | CATGATCAGCTGGGCCAAGACGGCTG | miR-187RP | T+CG+TGTCTTGTGTT | Identical overlapping |
| | | CAAC | | SEQ ID NO:242 | sequence, ends differ |
| | | SEQ ID NO:241 | | | |
| miR-188 | miR-188GSP | CATGATCAGCTGGGCCAAGAACCCTC | miR-188RP | C+AT+CCCTTGCATGG | Identical |
| | | CACC | | SEQ ID NO:244 | |
| | | SEQ ID NO:243 | | | |
| miR-189 | miR-189GSP2 | CATGATCAGCTGGGCCAAGAACTGAT | miR-189RP | G+TG+CCTAGTGAGCT | Identical |
| | | ATC | | SEQ ID NO:246 | |
| | | SEQ ID NO:245 | | | |
| miR-190 | miR-190GSP9 | CATGATCAGCTGGGCCAAGAACCTAA | miR-190RP4 | T+GA+TA+TGTTTGATATAT | Identical |
| | | TAT | | TAG | |
| | | SEQ ID NO:247 | | SEQ ID NO:248 | |
| miR-191 | miR-191GSP2 | CATGATCAGCTGGGCCAAGAAGCTGC | miR-191RP2 | C+AA+CGGAATCCCAAAAG | Identical |
| | | TTT | | SEQ ID NO:250 | |
| | | SEQ ID NO:249 | | | |
| miR-192 | miR-192GSP2 | CATGATCAGCTGGGCCAAGAGGCTGT | miR-192RP | C+TGA+CCTATGAATTGAC | Identical overlapping |
| | | CAA | | SEQ ID NO:252 | sequence, ends differ |
| | | SEQ ID NO:251 | | | |
| miR-193 | miR-193GSP9 | CATGATCAGCTGGGCCAAGACTGGGA | miR-193RP2 | AA+CT+GGCCTACAAAG | Identical |
| | | CTT | | SEQ ID NO:254 | |
| | | SEQ ID NO:253 | | | |
| miR-194 | mir-194GSP8 | CATGATCAGCTGGGCCAAGATCCACA | mir194RP | TG+TAA+CAGCAACTCCA | Identical |
| | | TG | | SEQ ID NO:256 | |
| | | SEQ ID NO:255 | | | |
| miR-195 | miR-195GSP9 | CATGATCAGCTGGGCCAAGAGCCAAT | miR-195RP3 | T+AG+CAG+CACAGAAATA | Identical |
| | | ATT | | SEQ ID NO:258 | |
| | | SEQ ID NO:257 | | | |
| miR-196a | miR-196aGSP | CATGATCAGCTGGGCCAAGACCAACA | miR-196aRP | TA+GG+TAGTTTCATGTTG | Identical |
| | | ACAT | | SEQ ID NO:262 | |
| | | SEQ ID NO:261 | | | |
| miR-196b | miR-196bGSP | CATGATCAGCTGGGCCAAGACCAACA | miR-196bRP | TA+GGT+AGTTTCCTGT | Identical |
| | | ACAG | | SEQ ID NO:260 | |
| | | SEQ ID NO:259 | | | |
| miR-199a* | miR-199a*GSP2 | CATGATCAGCTGGGCCAAGAAACCAA | miR-199a*RP | T+AC+AGTAGTCTGCAC | Identical |
| | | TGT | | SEQ ID NO:268 | |
| | | SEQ ID NO:267 | | | |
| miR-199a | miR-199aGSP2 | CATGATCAGCTGGGCCAAGAGAACAG | miR-199aRP | C+CC+AGTGTTCAGAC | Identical |
| | | GTA | | SEQ ID NO:270 | |
| | | SEQ ID NO:269 | | | |
| miR-199b | miR-199bGSP | CATGATCAGCTGGGCCAAGAGAACAG | miR-199bRP | C+CC+AGTGTTTAGAC | one or more base |
| | | GTAG | | SEQ ID NO:272 | pairs differ |
| | | SEQ ID NO:475 | | | |
| miR-200a | miR-200aGSP2 | CATGATCAGCTGGGCCAAGAACATCG | miR-200aRP | TAA+CAC+TGTCTGGT | Identical |
| | | TTA | | SEQ ID NO:274 | |
| | | SEQ ID NO:273 | | | |
| miR-200b | miR-200bGSP2 | CATGATCAGCTGGGCCAAGAGTCATC | miR-200bRP | TAATA+CTG+CCTGGTAAT | Identical |
| | | ATT | | SEQ ID NO:276 | |
| | | SEQ ID NO:275 | | | |
| miR-203 | miR-203GSP2 | CATGATCAGCTGGGCCAAGACTAGTG | miR-203RP | G+TG+AAATGTTTAGGACC | Identical overlapping |
| | | GTC | | SEQ ID NO:280 | sequence, ends differ |
| | | SEQ ID NO:279 | | | |
| miR-204 | miR-204GSP2 | CATGATCAGCTGGGCCAAGAAGGCAT | miR-204RP | T+TC+CCTTTGTCATCC | Identical overlapping |
| | | AGG | | SEQ ID NO:282 | sequence, ends differ |
| | | SEQ ID NO:281 | | | |
| miR-205 | miR-205GSP | CATGATCAGCTGGGCCAAGACAGACT | miR-205RP | T+CCTT+CATTCCACC | Identical |
| | | CCGG | | SEQ ID NO:284 | |
| | | SEQ ID NO:283 | | | |
| miR-206 | mir-206GSP7 | CATGATCAGCTGGGCCAAGACCACA | miR-206RP | T+G+GAA+TGTAAGGAAGTGT | Identical |
| | | CA | | SEQ ID NO:286 | |
| | | SEQ ID NO:285 | | | |
| miR-208 | miR-208_GSP13 | CATGATCAGCTGGGCCAAGAACAAGC | miR-208_RP4 | ATAA+GA+CG+AGCAAAAAG | Identical |
| | | TTTTTGC | | SEQ ID NO:288 | |
| | | SEQ ID NO:287 | | | |
| miR-210 | miR-210GSP | CATGATCAGCTGGGCCAAGATCAGCC | miR-210RP | C+TG+TGCGTGTGACA | Identical |
| | | GCTG | | SEQ ID NO:290 | |
| | | SEQ ID NO:289 | | | |
| miR-211 | miR-211GSP2 | CATGATCAGCTGGGCCAAGAAGGCAA | miR-211RP | T+TC+CCTTTGTCATCC | one or more base |
| | | AGG | | SEQ ID NO:292 | pairs differ |
| | | SEQ ID NO:491 | | | |
| miR-212 | miR-212GSP9 | CATGATCAGCTGGGCCAAGAGGCCGT | miR-212RP2 | T+AA+CAGTCTCCAGTCA | Identical |
| | | GAC | | SEQ ID NO:294 | |
| | | SEQ ID NO:293 | | | |
| miR-213 | miR-213GSP | CATGATCAGCTGGGCCAAGAGGTACA | miR-213RP | A+CC+ATCGACCGTTG | Identical |
| | | ATCA | | SEQ ID NO:296 | |
| | | SEQ ID NO:295 | | | |
| miR-214 | miR-214GSP | CATGATCAGCTGGGCCAAGACTGCCT | miR-214RP | A+CA+GCAGGCACAGA | Identical |
| | | GTCT | | SEQ ID NO:298 | |
| | | SEQ ID NO:297 | | | |
| miR-215 | miR-215GSP2 | CATGATCAGCTGGGCCAAGAGTCTGT | miR-215RP | A+TGA+CCTATCATTTGAC | one or more base |
| | | CAA | | SEQ ID NO:469 | pairs differ |
| | | SEQ ID NO:299 | | | |
| miR-216 | miR-216GSP9 | CATGATCAGCTGGGCCAAGACACAGT | mir-216RP | TAA+TCT+CAGCTGGCA | Identical |
| | | TGC | | SEQ ID NO:302 | |
| | | SEQ ID NO:301 | | | |
| miR-217 | miR-217GSP2 | CATGATCAGCTGGGCCAAGAATCCAG | miR-217RP2 | T+AC+TGCATCAGGAACTGA | one or more base |
| | | TCA | | SEQ ID NO:304 | pairs differ |
| | | SEQ ID NO:481 | | | |
| miR-218 | miR-218GSP2 | CATGATCAGCTGGGCCAAGAACATGG | miR-218RP | TTG+TGCTT+GATCTAAC | Identical |
| | | TTA | | SEQ ID NO:306 | |
| | | SEQ ID NO:305 | | | |
| miR-221 | miR-221GSP9 | CATGATCAGCTGGGCCAAGAGAAACC | miR-221RP | A+GC+TACATTCTCTGC | Identical overlapping |
| | | CAG | | SEQ ID NO:310 | sequence, ends differ |
| | | SEQ ID NO:309 | | | |
| miR-222 | miR-222GSP8 | CATGATCAGCTGGGCCAAGAGAGACC | miR-222RP | A+GC+TACATCTGGCT | Identical |
| | | CA | | SEQ ID NO:312 | |
| | | SEQ ID NO:311 | | | |
| miR-223 | miR-223GSP | CATGATCAGCTGGGCCAAGAGGGGTA | miR-223RP | TG+TC+AGTTTGTCAAA | Identical |
| | | TTTG | | SEQ ID NO:314 | |
| | | SEQ ID NO:313 | | | |
| miR-224 | miR-224GSP8 | CATGATCAGCTGGGCCAAGATAAACG | miR-224RP2 | C+AAG+TCACTAGTGGTT | Identical overlapping |
| | | GA | | SEQ ID NO:316 | sequence, ends differ |
| | | SEQ ID NO:315 | | | |
| miR-296 | miR-296GSP9 | CATGATCAGCTGGGCCAAGAACAGGA | miR-296RP2 | A+GG+GCCCCCCCTCAA | Identical |
| | | TTG | | SEQ ID NO:318 | |
| | | SEQ ID NO:317 | | | |
| miR-299 | miR-299GSP9 | CATGATCAGCTGGGCCAAGAATGTAT | miR-299RP | T+GG+TTTACCGTGCC | Identical |
| | | GTG | | SEQ ID NO:320 | |
| | | SEQ ID NO:319 | | | |
| miR-301 | miR-301GSP | CATGATCAGCTGGGCCAAGAGCTTTG | miR-301RP | C+AG+TGCAATAGTATTGT | Identical |
| | | ACAA | | SEQ ID NO:322 | |
| | | SEQ ID NO:321 | | | |
| miR-302a | miR-302aGSP | CATGATCAGCTGGGCCAAGATCACCA | miR-302aRP | T+AAG+TGCTTCCATGT | Identical |
| | | AAAC | | SEQ ID NO:326 | |
| | | SEQ ID NO:325 | | | |
| miR-320 | miR-320_GSP8 | CATGATCAGCTGGGCCAAGATTCGCC | miR-320_RP3 | AAAA+GCT+GGGTTGAGAGG | Identical |
| | | CT | | SEQ ID NO:338 | |
| | | SEQ ID NO:337 | | | |
| miR-323 | miR-323GSP | CATGATCAGCTGGGCCAAGAAGAGGT | miR-323RP | G+CA+CATTACACGGT | Identical |
| | | CGAC | | SEQ ID NO:340 | |
| | | SEQ ID NO:339 | | | |
| miR-324- | miR-324- | CATGATCAGCTGGGCCAAGACCAGCA | miR-324- | C+CA+CTGCCCCAGGT | Identical |
| 3p | 3pGSP | GCAC | 3pRP | SEQ ID NO:342 | |
| | | SEQ ID NO:341 | | | |
| miR-324- | miR-324- | CATGATCAGCTGGGCCAAGAACACCA | miR-324- | C+GC+ATCCCCTAGGG | Identical overlapping |
| 5p | 5pGSP | ATGC | 5pRP | SEQ ID NO:344 | sequence, ends differ |
| | | SEQ ID NO:343 | | | |
| miR-325 | miR-325GSP | CATGATCAGCTGGGCCAAGAACACTT | miR-325RP | C+CT+AGTAGGTGCTC | one or more base |
| | | ACTG | | SEQ ID NO:476 | pairs differ |
| | | SEQ ID NO:345 | | | |
| miR-326 | miR-326GSP | CATGATCAGCTGGGCCAAGACTGGAG | miR-326RP | C+CT+CTGGGCCCTTC | Identical overlapping |
| | | GAAG | | SEQ ID NO:348 | sequence, ends differ |
| | | SEQ ID NO:347 | | | |
| miR-328 | miR-328GSP | CATGATCAGCTGGGCCAAGAACGGAA | miR-328RP | C+TG+GCCCTCTCTGC | Identical |
| | | GGGC | | SEQ ID NO:350 | |
| | | SEQ ID NO:349 | | | |
| miR-330 | miR-330GSP | CATGATCAGCTGGGCCAAGATCTCTG | miR-330RP | G+CA+AAGCACAGGGC | one or more base |
| | | CAGG | | SEQ ID NO:478 | pairs differ |
| | | SEQ ID NO:351 | | | |
| miR-331 | miR-331GSP | CATGATCAGCTGGGCCAAGATTCTAG | miR-331RP | G+CC+CCTGGGCCTAT | Identical |
| | | GATA | | SEQ ID NO:354 | |
| | | SEQ ID NO:353 | | | |
| miR-337 | miR-337GSP | CATGATCAGCTGGGCCAAGAAAAGGC | miR-337RP | T+TC+AGCTCCTATATG | one or more base |
| | | ATCA | | SEQ ID NO:490 | pairs differ |
| | | SEQ ID NO:355 | | | |
| miR-338 | miR-338GSP | CATGATCAGCTGGGCCAAGATCAACA | miR-338RP2 | T+CC+AGCATCAGTGATTT | Identical |
| | | AAAT | | SEQ ID NO:358 | |
| | | SEQ ID NO:357 | | | |
| miR-339 | miR-339GSP9 | CATGATCAGCTGGGCCAAGATGAGCT | miR-339RP2 | T+CC+CTGTCCTCCAGG | Identical |
| | | CCT | | SEQ ID NO:360 | |
| | | SEQ ID NO:359 | | | |
| miR-340 | miR-340GSP | CATGATCAGCTGGGCCAAGAGGCTAT | miR-340RP | TC+CG+TCTCAGTTAC | Identical |
| | | AAAG | | SEQ ID NO:362 | |
| | | SEQ ID NO:361 | | | |
| miR-342 | miR-342GSP3 | CATGATCAGCTGGGCCAAGAGACGGG | miR-342RP | T+CT+CACACAGIAAATCG | Identical |
| | | TG | | SEQ ID NO:364 | |
| | | SEQ ID NO:363 | | | |
| miR-345 | miR-345GSP | CATGATCAGCTGGGCCAAGAGCACTG | miR-345RP | T+GC+TGACCCCTAGT | one or more base |
| | | GACT | | SEQ ID NO:485 | pairs differ |
| | | SEQ ID NO:484 | | | |
| miR-346 | miR-346GSP | CATGATCAGCTGGGCCAAGAAGAGGC | miR-346RP | T+GT+CTGCCCGAGTG | one or more base |
| | | AGGC | | SEQ ID NO:488 | pairs differ |
| | | SEQ ID NO:367 | | | |
| miR-363 | miR-363GSP10 | CATGATCAGCTGGGCCAAGATACAGA | miR-363RP | AAT+TG+CAC+GGTATCC | Identical |
| | | TGGA | | SEQ ID NO:370 | |
| | | SEQ ID NO:369 | | | |
| miR-370 | miR-370GSP | CATGATCAGCTGGGCCAAGACCAGGT | miR-370RP | G+CC+TGCTGGGGTGG | Identical overlapping |
| | | TCCA | | SEQ ID NO:376 | sequence, ends differ |
| | | SEQ ID NO:375 | | | |
| miR-375 | miR-375GSP | CATGATCAGCTGGGCCAAGATCACGC | miR-375RP | TT+TG+TTCGTTCGGC | Identical |
| | | GAGC | | SEQ ID NO:388 | |
| | | SEQ ID NO:387 | | | |
| miR-376a | miR-376aGSP3 | CATGATCAGCTGGGCCAAGAACGTGG | miR-376aRP2 | A+TCGTAGA+GGAAAATCCAC | one or more base |
| | | AT | | SEQ ID NO:468 | pairs differ |
| | | SEQ ID NO:467 | | | |
| miR-378 | miR-378GSP | CATGATCAGCTGGGCCAAGAACACAG | miR-378RP | C+TC+CTGACTCCAGG | Identical |
| | | GACC | | SEQ ID NO:392 | |
| | | SEQ ID NO:391 | | | |
| miR-379 | miR-379_GSP7 | CATGATCAGCTGGGCCAAGATACGT | miR-379RP2 | T+GGT+AGACTATGGAACG | Identical overlapping |
| | | TC | | SEQ ID NO:394 | sequence, ends differ |
| | | SEQ ID NO:393 | | | |
| miR-380- | miR-380-5pGSP | CATGATCAGCTGGGCCAAGAGCGCAT | miR-380- | T+GGT+TGACCATAGA | Identical |
| 5p | | GTTC | 5pRP | SEQ ID NO:396 | |
| | | SEQ ID NO:395 | | | |
| miR-380- | miR-380-3pGSP | CATGATCAGCTGGGCCAAGAAAGATG | miR-380- | TA+TG+TAGTATGGTCCACA | one or more base |
| 3p | | TGGA | 3pRP | SEQ ID NO:483 | pairs differ |
| | | SEQ ID NO:395 | | | |
| miR-381 | miR-381GSP2 | CATGATCAGCTGGGCCAAGAACAGAG | miR-381RP2 | TATA+CAA+GGGCAAGCT | Identical |
| | | AGC | | SEQ ID NO:400 | |
| | | SEQ ID NO:399 | | | |
| miR-382 | miR-382GSP | CATGATCAGCTGGGCCAAGACGAATC | miR-382RP | G+AA+GTTGTTCGTGGT | Identical |
| | | CACC | | SEQ ID NO:402 | |
| | | SEQ ID NO:401 | | | |
| miR-383 | miR-383GSP | CATGATCAGCTGGGCCAAGAAGCCAC | miR-383RP2 | A+GATC+AGAAGGTGACTGT | one or more base |
| | | AGTC | | SEQ ID NO:466 | pairs differ |
| | | SEQ ID NO:465 | | | |
| miR-384 | miR-384_GSP9 | CATGATCAGCTGGGCCAAGATGTGAA | miR-384_RP5 | ATT+CCT+AG+AAATTGTTC | one or more base |
| | | CAA | | SEQ ID NO:471 | pairs differ |
| | | SEQ ID NO:470 | | | |
| miR-410 | miR-410GSP9 | CATGATCAGCTGGGCCAAGAACAGGC | miR-410RP | AA+TA+TAA+CA+CAGATGGC | Identical |
| | | CAT | | SEQ ID NO:406 | |
| | | SEQ ID NO:405 | | | |
| miR-412 | miR-412GSP10 | CATGATCAGCTGGGCCAAGAACGGCT | miR-412RP | A+CTT+CACCTGGTCCACTA | Identical |
| | | AGTG | | SEQ ID NO:408 | |
| | | SEQ ID NO:407 | | | |
| miR-424 | miR-424GSP | CATGATCAGCTGGGCCAAGATCCAAA | miR-424RP2 | C+AG+CAGCAATTCATGTTTT | one or more base |
| | | ACAT | | SEQ ID NO:414 | pairs differ |
| | | SEQ ID NO:474 | | | |
| miR-425 | miR-425GSP | CATGATCAGCTGGGCCAAGAGGCGGA | miR-425RP | A+TC+GGGAATGTCGT | Identical |
| | | CACG | | SEQ ID NO:418 | |
| | | SEQ ID NO:417 | | | |
| miR-429 | miR-429_GSP11 | CATGATCAGCTGGGCCAAGAACGGCA | miR-429RP5 | T+AATAC+T+TCTGGTAATG | one or more base |
| | | TTACC | | SEQ ID NO: 480 | pairs differ |
| | | SEQ ID NO:479 | | | |
| miR-431 | miR-431GSP10 | CATGATCAGCTGGGCCAAGATGCATG | miR-431RP | T+GT+CTTGCAGGCCG | Identical overlapping |
| | | ACGG | | SEQ ID NO: 422 | sequence, ends differ |
| | | SEQ ID NO:421 | | | |
| miR-448 | miR-448GSP | CATGATCAGCTGGGCCAAGAATGGGA | miR-448RP | TTG+CATA+TGTAGGATG | Identical |
| | | CATC | | SEQ ID NO: 424 | |
| | | SEQ ID NO:423 | | | |
| miR-449 | miR-449GSP10 | CATGATCAGCTGGGCCAAGAACCAGC | miR-449RP2 | T+GG+CAGTGTATTGTTAGC | Identical |
| | | TAAC | | SEQ ID NO:426 | |
| | | SEQ ID NO:425 | | | |
| miR-450 | miR-450GSP | CATGATCAGCTGGGCCAAGATATTAG | miR-450RP | TTTT+TG+CGATGTGTT | Identical |
| | | GAAC | | SEQ ID NO:428 | |
| | | SEQ ID NO:427 | | | |
| miR-451 | miR-451GSP10 | CATGATCAGCTGGGCCAAGAAAACTC | miR-451RP | AAA+CCG+TTA+CCATTAC | Identical overlapping |
| | | AGTA | | TGA | sequence, ends differ |
| | | SEQ ID NO:429 | | SEQ ID NO:430 | |
| let7a | let7a-GSP2 | CATGATCAGCTGGGCCAAGAAACTAT | let7a-RP | T+GA+GGTAGTAGGTTG | Identical overlapping |
| | | AC | | SEQ ID NO:432 | sequence, ends differ |
| | | SEQ ID NO:431 | | | |
| let7b | let7b-GSP2 | CATGATCAGCTGGGCCAAGAAACCAC | let7b-RP | T+GA+GGTAGTAGGTTG | Identical |
| | | AC | | SEQ ID NO:432 | |
| | | SEQ ID NO:433 | | | |
| let7c | let7c-GSP2 | CATGATCAGCTGGGCCAAGAAACCAT | let7c-RP | T+GA+GGTAGTAGGTTG | Identical |
| | | AC | | SEQ ID NO:432 | |
| | | SEQ ID NO:434 | | | |
| let7d | let7d-GSP2 | CATGATCAGCTGGGCCAAGAACTATG | let7d-RP | A+GA+GGTAGTAGGTTG | Identical |
| | | CA | | SEQ ID NO:436 | |
| | | SEQ ID NO:435 | | | |
| let7e | let7e-GSP2 | CATGATCAGCTGGGCCAAGAACTATA | let7e-RP | T+GA+GGTAGGAGGTTG | Identical |
| | | CA | | SEQ ID NO:438 | |
| | | SEQ ID NO:437 | | | |
| let7f | let7f-GSP2 | CATGATCAGCTGGGCCAAGAAACTAT | let7f-RP | T+GA+GGTAGTAGATTG | Identical overlapping |
| | | AC | | SEQ ID NO:440 | sequence, ends differ |
| | | SEQ ID NO:439 | | | |
| let7g | let7g-GSP2 | CATGATCAGCTGGGCCAAGAACTGTA | let7g-RP | T+GA+GGTAGTAGTTTG | Identical |
| | | CA | | SEQ ID NO:442 | |
| | | SEQ ID NO:441 | | | |
| let7i | let7i-GSP2 | CATGATCAGCTGGGCCAAGAACAGCA | let7i-RP | T+GA+GGTAGTAGTTTG | Identical |
| | | CA | | SEQ ID NO:444 | |
| | | SEQ ID NO:443 | | | |

### EXAMPLE 5

This Example describes the detection and analysis of expression profiles for three microRNAs in total RNA isolated from twelve different tissues using methods in accordance with an embodiment of the present invention.

Methods: Quantitative analysis of miR-1, miR-124 and miR-150 microRNA templates was determined using 0.5 μg of First Choice total RNA (Ambion, Inc.) per 10 μl primer extension reaction isolated from the following tissues: brain, heart, intestine, kidney, liver, lung, lymph, ovary, skeletal-muscle, spleen, thymus and uterus. The primer extension enzyme and quantitative PCR reactions were carried out as described above in EXAMPLE 3, using the following PCR primers:

| | |
|---|---|
| miR-1 template: | |
| extension primer: | |
| CATGATCAGCTGGGCCAAGATACATACTTC | (SEQ ID NO: 47) |
| reverse primer: | |
| T+G+GAA+TG+ATAAAGAAGT | (SEQ ID NO: 48) |
| forward primer: | |
| CATGATCAGCTGGGCCAAGA | (SEQ ID NO: 13) |
| miR-124 template: | |
| extension primer: | |
| CATGATCAGCTGGGCCAAGATGGCATTCAC | (SEQ ID NO: 149) |
| reverse primer: | |
| T+TA+AGGCACGCGGT | (SEQ ID NO: 150) |
| forward primer: | |
| CATGATCAGCTGGGCCAAGA | (SEQ ID NO: 13) |
| miR-150 template: | |
| extension primer: | |
| CATGATCAGCTGGGCCAAGACACTGGTA | (SEQ ID NO: 213) |
| reverse primer: | |
| T+CT+CCCAACCCTTG | (SEQ ID NO: 214) |
| forward primer: | |
| CATGATCAGCTGGGCCAAGA | (SEQ ID NO: 13) |

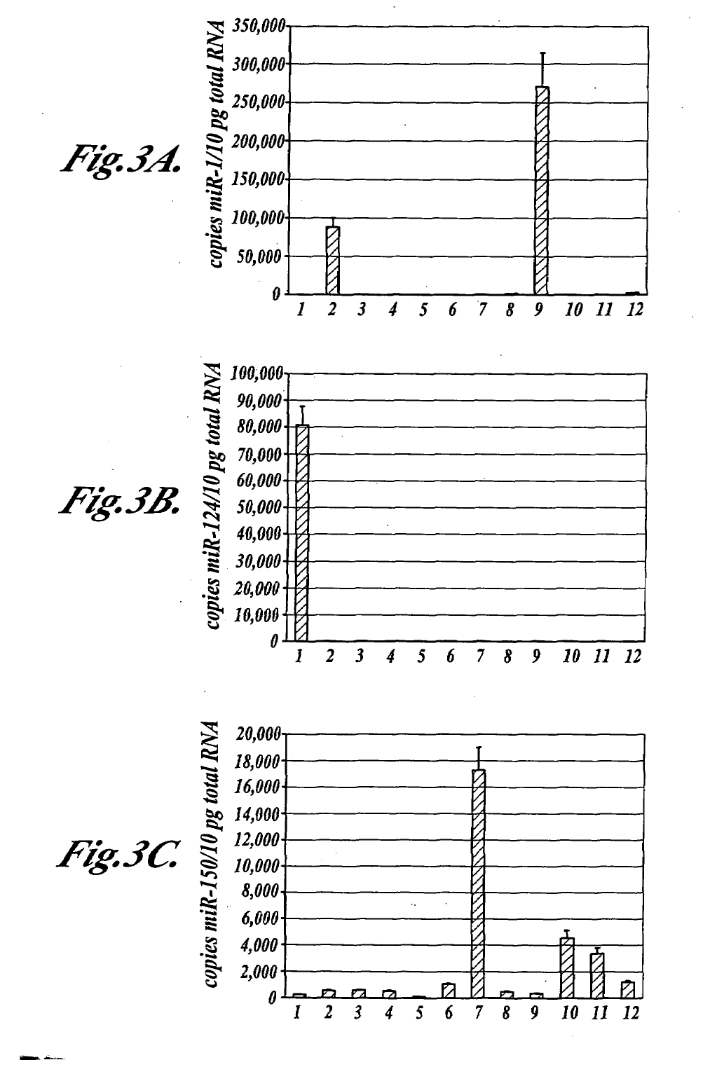

Results: The expression profiles for miR-1, miR-124 and miR-150 are shown in FIGS. 3A, 3B, and 3C, respectively. The data in FIGS. 3A-3C are presented in units of microRNA copies per 10 pg of total RNA (y-axis). These units were chosen since human cell lines typically yield ≦10 pg of total RNA per cell. Hence the data shown are estimates of microRNA copies per cell. The numbers on the x-axis correspond to the following tissues: (1) brain, (2) heart, (3) intestine, (4) kidney, (5) liver, (6) lung, (7) lymph, (8) ovary, (9) skeletal muscle, (10) spleen, (11) thymus and (12) uterus.

Consistent with previous reports, very high levels of striated muscle-specific expression were found for miR-1 (as shown in FIG. 3A), and high levels of brain expression were found for miR-124 (as shown in FIG. 3B) (see Lagos-Quintana et al., RNA 9:175-179, 2003). Quantitative analysis reveals that these microRNAs are present at tens to hundreds of thousands of copies per cell. These data are in agreement with quantitative Northern blot estimates of miR-1 and miR-124 levels (see Lim et al., Nature 433:769-773, 2005). As shown in FIG. 3C, miR-150 was found to be highly expressed in the immune-related lymph node, thymus and spleen samples which is also consistent with previous findings (see Baskerville et al., RNA 11:241-247, 2005).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for amplifying a microRNA molecule to produce DNA molecules, the method comprising the steps of:
(a) producing a first DNA molecule that is complementary to a target microRNA molecule using primer extension; and
(b) amplifying the first DNA molecule to produce amplified DNA molecules using a universal forward primer and a reverse primer.

2. The method of claim 1, wherein at least one of the universal forward primer and the reverse primer comprises at least one locked nucleic acid molecule.

3. A method of claim 1 wherein the primer extension uses an extension primer having a length in the range of from 10 to 100 nucleotides.

4. A method of claim 1 wherein the primer extension uses an extension primer having a length in the range of from 20 to 35 nucleotides.

5. A method of claim 1 wherein the extension primer comprises a first portion that hybridizes to a portion of the microRNA molecule.

6. A method of claim 5 wherein the first portion has a length in the range of from 3 to 25 nucleotides.

7. A method of claim 5 wherein the extension primer comprises a second portion.

8. A method of claim 7 wherein the second portion has a length of from 18 to 25 nucleotides.

9. A method of claim 7 wherein the second portion has a nucleic acid sequence comprising the nucleic acid sequence of SEQ ID NO:1.

10. A method of claim 1 wherein the universal forward primer has a length in the range of from 16 nucleotides to 100 nucleotides.

11. A method of claim 1 wherein the universal forward primer consists of the nucleic acid sequence set forth in SEQ ID NO:13.

12. A method of claim 7 wherein the universal forward primer hybridizes to the complement of the second portion of the extension primer.

13. A method of claim 2 wherein the universal forward primer comprises at least one locked nucleic acid molecule.

14. A method of claim 13 wherein the universal forward primer comprises from 1 to 25 locked nucleic acid molecules.

15. A method of claim 1 wherein the reverse primer has a length in the range of from 10 nucleotides to 100 nucleotides.

16. A method of claim 2 wherein the reverse primer comprises at least one locked nucleic acid molecule.

17. A method of claim 16 wherein the reverse primer comprises from 1 to 25 locked nucleic acid molecules.

18. A method of claim 1 wherein the reverse primer is selected to specifically hybridize to a DNA molecule complementary to a selected microRNA molecule under defined hybridization conditions.

19. A method of claim 1 further comprising the step of measuring the amount of amplified DNA molecules.

20. A method of claim 1 wherein amplification is achieved by multiple successive PCR reactions.

21. A method for measuring the amount of a target microRNA in a sample from a living organism, the method comprising the step of measuring the amount of a target microRNA molecule in a multiplicity of different cell types within a living organism, wherein the amount of the target microRNA molecule is measured by a method comprising the steps of:
(1) producing a first DNA molecule complementary to the target microRNA molecule in the sample using primer extension;
(2) amplifying the first DNA molecule to produce amplified DNA molecules using a universal forward and a reverse primer; and
(3) measuring the amount of the amplified DNA molecules.

22. The method of claim 21, wherein at least one of the universal forward primer and the reverse primer comprises at least one locked nucleic acid molecule.

23. The method of claim 21, wherein the amount of the amplified DNA molecules are measured using fluorescence-based quantitative PCR.

24. The method of claim 21, wherein the amount of the amplified DNA molecules are measured using SYBR green dye.

25. A kit for detecting at least one mammalian target microRNA comprising at least one primer set specific for the detection of a target microRNA, the primer set comprising:
(1) an extension primer for producing a cDNA molecule complementary to a target microRNA, the extension primer comprising a first portion that hybridizes to a target microRNA and a second portion having a hybridization sequence for a universal forward PCR primer;
(2) a universal forward PCR primer for amplifying the cDNA molecule, comprising a sequence selected to hybridize to the hybridization sequence on the extension primer; and
(3) a reverse PCR primer for amplifying the cDNA molecule, comprising a sequence selected to hybridize to a portion of the cDNA molecule.

26. The kit according to claim 25, wherein at least one of the universal forward and reverse PCR primers includes at least one locked nucleic acid molecule.

27. The kit according to claim 25, wherein the extension primer has a length in the range of from 10 to 100 nucleotides.

28. The kit according to claim 25, wherein the first portion of the extension primer has a length in the range of from 3 to 25 nucleotides.

29. The kit according to claim 25, wherein the second portion of the extension primer has a length in the range of from 18 to 25 nucleotides.

30. The kit according to claim 25, wherein the second portion of the extension primer has a nucleic acid sequence comprising the nucleic acid sequence of SEQ ID NO: 1.

31. The kit according to claim 25, wherein the universal forward PCR primer has a length in the range of from 16 to 100 nucleotides.

32. The kit according to claim 25, wherein the universal forward primer consists of the nucleic acid sequence set forth in SEQ ID NO: 13.

33. The kit according to claim 25, wherein the reverse PCR primer has a length in the range of from 10 to 100 nucleotides.

34. The kit according to claim 25, wherein the reverse PCR primer comprises from 1 to 25 locked nucleic acid molecules.

35. The kit according to claim 25, wherein the at least one mammalian target microRNA is a human microRNA.

36. The kit according to claim 35, wherein the at least one target microRNA is selected from the group consisting of miR-1, miR-7, miR-9*, miR-10a, miR-10b, miR-15a, miR-15b, miR-16, miR-17-3p, miR-17-5p, miR-18, miR-19a, miR-19b, miR-20, miR-21, miR-22, miR-23a, miR-23b, miR-24, miR-25, miR-26a, miR-26b, miR-27a, miR-28, miR-29a, miR-29b, miR-29c, miR-30a-5p, miR-30b, miR-30c, miR-30d, miR-30e-5p, miR-30e-3p, miR-31, miR-32, miR-33, miR-34a, miR-34b, miR-34c, miR-92, miR-93, miR-95, miR-96, miR-98, miR-99a, miR-99b, miR-100, miR-101, miR-103, miR-105, miR-106a, miR-107, miR-122, miR-122a, miR-124, miR-124, miR-124a, miR-125a, miR-125b, miR-126, miR-126*, miR-127, miR-128a, miR-128b, miR-129, miR-130a, miR-130b, miR-132, miR-133a, miR-133b, miR-134, miR-135a, miR-135b, miR-136, miR-137, miR-138, miR-139, miR-140, miR-141, miR-142-3p, miR-143, miR-144, miR-145, miR-146, miR-147, miR-148a, miR-148b, miR-149, miR-150, miR-151, miR-152, miR-153, miR-154*, miR-154, miR-155, miR-181a, miR-181b, miR-181c, miR-182*, miR-182, miR-183, miR-184, miR-185, miR-186, miR-187, miR-188, miR-189, miR-190, miR-191, miR-192, miR-193, miR-194, miR-195, miR-196a, miR-196b, miR-197, miR-198, miR-199a*, miR-199a, miR-199b, miR-200a, miR-200b, miR-200c, miR-202, miR-203, miR-204, miR-205, miR-206, miR-208, miR-210, miR-211, miR-212, miR-213, miR-213, miR-214, miR-215, miR-216, miR-217, miR-218, miR-220, miR-221, miR-222, miR-223, miR-224, miR-296, miR-299, miR-301, miR-302a*, miR-302a, miR-302b*, miR-302b, miR-302d, miR-302c*, miR-302c, miR-320, miR-323, miR-324-3p, miR-324-5p, miR-325, miR-326, miR-328, miR-330, miR-331, miR-337, miR-338, miR-339, miR-340, miR-342, miR-345, miR-346, miR-363, miR-367, miR-368, miR-370, miR-371, miR-372, miR-373*, miR-373, miR-374, miR-375, miR-376b, miR-378, miR-379, miR-380-5p, miR-380-3p, miR-381, miR-382, miR-383, miR-410, miR-412, miR-422a, miR-422b, miR-423, miR-424, miR-425, miR-429, miR-431, miR-448, miR-449, miR-450, miR-451, let7a, let7b, let7c, let7d, let7e, let7f, let7g, let7i, miR-376a, and miR-377.

37. The kit according to claim 35, wherein the at least one target microRNA is selected from the group consisting of: miR-1, miR-7, miR-10b, miR-26a, miR-26b, miR-29a, miR-30e-3p, miR-95, miR-107, miR-141, miR-143, miR-154*, miR-154, miR-155, miR-181a, miR-181b, miR-181c, miR-190, miR-193, miR-194, miR-195, miR-202, miR-206, miR-208, miR-212, miR-221, miR-222, miR-224, miR-296, miR-299, miR-302c*, miR-302c, miR-320, miR-339, miR-363, miR-376b, miR-379, miR-410, miR-412, miR-424, miR-429, miR-431, miR-449, miR-451, let7a, let7b, let7c, let7d, let7e, let7f, let7g, and let7i.

38. The kit according to claim 25, wherein the at least one target microRNA is a murine microRNA.

39. A kit for detecting at least one mammalian microRNA comprising at least one oligonucleotide primer selected from the group consisting of SEQ ID NO: 2 to SEQ ID NO:499.

40. The kit according to claim 39 comprising at least one or more oligonucleotide primers selected from the group consisting of SEQ ID NOS: 47, 48, 49, 50, 55, 56, 81, 82, 83, 84, 91, 92, 103, 104, 123, 124, 145, 146, 193, 194, 197, 198, 221, 222, 223, 224, 225, 226, 227, 228, 229, 230, 239, 240, 247, 248, 253, 254, 255, 256, 257, 258, 277, 278, 285, 286, 287, 288, 293, 294, 301, 302, 309, 310, 311, 312, 315, 316, 317, 318, 319, 320, 333, 334, 335, 336, 337, 338, 359, 360, 369, 370, 389, 390, 393, 394, 405, 406, 407, 408, 415, 416, 419, 420, 421, 422, 425, 426, 429, 430, 431, 432, 433, 434, 435, 436, 437, 438, 439, 440, 441, 442, 443, 444, 461 and 462.

41. An oligonucleotide primer for detecting a human microRNA selected from the group consisting of SEQ ID NO: 2 to SEQ ID NO: 499.

42. An oligonucleotide primer according to claim 41, wherein the primer is selected from the group consisting of SEQ ID NO: 47, 48, 49, 50, 55, 56, 81, 82, 83, 84, 91, 92, 103, 104, 123, 124, 145, 146, 193, 194, 197, 198, 221, 222, 223, 224, 225, 226, 227, 228, 229, 230, 239, 240, 247, 248, 253, 254, 255, 256, 257, 258, 277, 278, 285, 286, 287, 288, 293, 294, 301, 302, 309, 310, 311, 312, 315, 316, 317, 318, 319, 320, 333, 334, 335, 336, 337, 338, 359, 360, 369, 370, 389, 390, 393, 394, 405, 406, 407, 408, 415, 416, 419, 420, 421, 422, 425, 426, 429, 430, 431, 432, 433, 434, 435, 436, 437, 438, 439, 440, 441, 442, 443, 444, 461 and 462.


SEQUENCE LISTING (499 sequences)

SEQ ID NO: 1 - DNA, 20 bp, Artificial Sequence
  Primer
catgatcagctgggccaaga

SEQ ID NO: 2 - DNA, 33 bp, Artificial Sequence
  Primer
catgatcagctgggccaagaaactatacaacct

SEQ ID NO: 3 - DNA, 31 bp, Artificial Sequence
  Primer
catgatcagctgggccaagatacatacttct

SEQ ID NO: 4 - DNA, 34 bp, Artificial Sequence
  Primer
catgatcagctgggccaagacacaaaccattatg

SEQ ID NO: 5 - DNA, 35 bp, Artificial Sequence
  Primer
catgatcagctgggccaagacgccaatatttacgt

SEQ ID NO: 6 - DNA, 31 bp, Artificial Sequence
  Primer
catgatcagctgggccaagatcaacatcagt

SEQ ID NO: 7 - DNA, 32 bp, Artificial Sequence
  Primer
catgatcagctgggccaagactgttcctgctg

SEQ ID NO: 8 - DNA, 36 bp, Artificial Sequence
  Primer
catgatcagctgggccaagaacaaacaccattgtca

SEQ ID NO: 9 - DNA, 36 bp, Artificial Sequence
  Primer
catgatcagctgggccaagatggcattcaccgcgtg

SEQ ID NO: 10 - DNA, 32 bp, Artificial Sequence
  Primer
catgatcagctgggccaagatgagctacagtg

SEQ ID NO: 11 - DNA, 36 bp, Artificial Sequence
  Primer
catgatcagctgggccaagaaagggattcctgggaa

SEQ ID NO: 12 - DNA, 33 bp, Artificial Sequence
  Primer
catgatcagctgggccaagacccctatcacgat

SEQ ID NO: 13 - DNA, 20 bp, Artificial Sequence
  Primer
catgatcagctgggccaaga

SEQ ID NO: 14 - DNA, 15 bp, Artificial Sequence
  Primer
tgaggtagtaggttg

SEQ ID NO: 15 - DNA, 17 bp, Artificial Sequence
  Primer
tggaatgtaaagaagta

SEQ ID NO: 16 - DNA, 15 bp, Artificial Sequence
  Primer
tagcagcacataatg

SEQ ID NO: 17 - DNA, 14 bp, Artificial Sequence
  Primer
tagcagcacgtaaa

SEQ ID NO: 18 - DNA, 17 bp, Artificial Sequence
  Primer
tagcttatcagactgat

SEQ ID NO: 19 - DNA, 14 bp, Artificial Sequence
  Primer
tggctcagttcagc

SEQ ID NO: 20 - DNA, 13 bp, Artificial Sequence
  Primer
tggagtgtgacaa

SEQ ID NO: 21 - DNA, 12 bp, Artificial Sequence
  Primer
ttaaggcacgcg

SEQ ID NO: 22 - DNA, 15 bp, Artificial Sequence
  Primer
tgagatgaagcactg

SEQ ID NO: 23 - DNA, 14 bp, Artificial Sequence
  Primer
gtccagttttccca

SEQ ID NO: 24 - DNA, 16 bp, Artificial Sequence
  Primer
ttaatgctaatcgtga

SEQ ID NO: 25 - DNA, 32 bp, Artificial Sequence
  Primer
catgatcagctgggccaagagccaatatttct

SEQ ID NO: 26 - DNA, 31 bp, Artificial Sequence
  Primer
catgatcagctgggccaagagccaatatttc

SEQ ID NO: 27 - DNA, 30 bp, Artificial Sequence
  Primer
catgatcagctgggccaagagccaatattt

SEQ ID NO: 28 - DNA, 29 bp, Artificial Sequence
  Primer
catgatcagctgggccaagagccaatatt

SEQ ID NO: 29 - DNA, 28 bp, Artificial Sequence
  Primer
catgatcagctgggccaagagccaatat

SEQ ID NO: 30 - DNA, 27 bp, Artificial Sequence
  Primer
catgatcagctgggccaagagccaata

SEQ ID NO: 31 - DNA, 26 bp, Artificial Sequence
  Primer
catgatcagctgggccaagagccaat

SEQ ID NO: 32 - DNA, 25 bp, Artificial Sequence
  Primer
catgatcagctgggccaagagccaa

SEQ ID NO: 33 - DNA, 24 bp, Artificial Sequence
  Primer
catgatcagctgggccaagagcca

SEQ ID NO: 34 - DNA, 23 bp, Artificial Sequence
  Primer
catgatcagctgggccaagagcc

SEQ ID NO: 35 - DNA, 32 bp, Artificial Sequence
  Primer
catgatcagctgggccaagagtctgtcaattc

SEQ ID NO: 36 - DNA, 31 bp, Artificial Sequence
  Primer
catgatcagctgggccaagagtctgtcaatt

SEQ ID NO: 37 - DNA, 30 bp, Artificial Sequence
  Primer
catgatcagctgggccaagagtctgtcaat

SEQ ID NO: 38 - DNA, 29 bp, Artificial Sequence
  Primer
catgatcagctgggccaagagtctgtcaa

SEQ ID NO: 39 - DNA, 28 bp, Artificial Sequence
  Primer
catgatcagctgggccaagagtctgtca

SEQ ID NO: 40 - DNA, 27 bp, Artificial Sequence
  Primer
catgatcagctgggccaagagtctgtc

SEQ ID NO: 41 - DNA, 26 bp, Artificial Sequence
  Primer
catgatcagctgggccaagagtctgt

SEQ ID NO: 42 - DNA, 25 bp, Artificial Sequence
  Primer
catgatcagctgggccaagagtctg

SEQ ID NO: 43 - DNA, 24 bp, Artificial Sequence
  Primer
catgatcagctgggccaagagtct

SEQ ID NO: 44 - DNA, 23 bp, Artificial Sequence
  Primer
catgatcagctgggccaagagtc

SEQ ID NO: 45 - DNA, 15 bp, Artificial Sequence
  Primer
tagcagcacagaaat

SEQ ID NO: 46 - DNA, 15 bp, Artificial Sequence
  Primer
atgacctatgaattg

SEQ ID NO: 47 - DNA, 30 bp, Artificial Sequence
  Primer
catgatcagctgggccaagatacatacttc

SEQ ID NO: 48 - DNA, 16 bp, Artificial Sequence
  Primer
tggaatgtaaagaagt

SEQ ID NO: 49 - DNA, 30 bp, Artificial Sequence
  Primer
catgatcagctgggccaagacaacaaaatc

SEQ ID NO: 50 - DNA, 18 bp, Artificial Sequence
  Primer
tggaagactagtgatttt

SEQ ID NO: 51 - DNA, 30 bp, Artificial Sequence
  Primer
catgatcagctgggccaagaactttcggtt

SEQ ID NO: 52 - DNA, 16 bp, Artificial Sequence
  Primer
taaagctagataaccg

SEQ ID NO: 53 - DNA, 30 bp, Artificial Sequence
  Primer
catgatcagctgggccaagacacaaattcg

SEQ ID NO: 54 - DNA, 15 bp, Artificial Sequence
  Primer
taccctgtagatccg

SEQ ID NO: 55 - DNA, 31 bp, Artificial Sequence
  Primer
catgatcagctgggccaagaacaaattcggt

SEQ ID NO: 56 - DNA, 16 bp, Artificial Sequence
  Primer
taccctgtagaaccga

SEQ ID NO: 57 - DNA, 30 bp, Artificial Sequence
  Primer
catgatcagctgggccaagacacaaaccat

SEQ ID NO: 58 - DNA, 15 bp, Artificial Sequence
  Primer
tagcagcacataatg

SEQ ID NO: 59 - DNA, 29 bp, Artificial Sequence
  Primer
catgatcagctgggccaagatgtaaacca

SEQ ID NO: 60 - DNA, 14 bp, Artificial Sequence
  Primer
tagcagcacatcat

SEQ ID NO: 61 - DNA, 29 bp, Artificial Sequence
  Primer
catgatcagctgggccaagacgccaatat

SEQ ID NO: 62 - DNA, 14 bp, Artificial Sequence
  Primer
tagcagcacgtaaa

SEQ ID NO: 63 - DNA, 30 bp, Artificial Sequence
  Primer
catgatcagctgggccaagaacaagtgcct

SEQ ID NO: 64 - DNA, 14 bp, Artificial Sequence
  Primer
actgcagtgaaggc

SEQ ID NO: 65 - DNA, 29 bp, Artificial Sequence
  Primer
catgatcagctgggccaagaactacctgc

SEQ ID NO: 66 - DNA, 16 bp, Artificial Sequence
  Primer
caaagtgcttacagtg

SEQ ID NO: 67 - DNA, 29 bp, Artificial Sequence
  Primer
catgatcagctgggccaagatcagttttg

SEQ ID NO: 68 - DNA, 15 bp, Artificial Sequence
  Primer
tgtgcaaatctatgc

SEQ ID NO: 69 - DNA, 30 bp, Artificial Sequence
  Primer
catgatcagctgggccaagatcagttttgc

SEQ ID NO: 70 - DNA, 14 bp, Artificial Sequence
  Primer
tgtgcaaatccatg

SEQ ID NO: 71 - DNA, 28 bp, Artificial Sequence
  Primer
catgatcagctgggccaagactacctgc

SEQ ID NO: 72 - DNA, 18 bp, Artificial Sequence
  Primer
taaagtgcttatagtgca

SEQ ID NO: 73 - DNA, 29 bp, Artificial Sequence
  Primer
catgatcagctgggccaagatcaacatca

SEQ ID NO: 74 - DNA, 18 bp, Artificial Sequence
  Primer
tagcttatcagactgatg

SEQ ID NO: 75 - DNA, 30 bp, Artificial Sequence
  Primer
catgatcagctgggccaagaggaaatccct

SEQ ID NO: 76 - DNA, 14 bp, Artificial Sequence
  Primer
atcacattgccagg

SEQ ID NO: 77 - DNA, 30 bp, Artificial Sequence
  Primer
catgatcagctgggccaagaggtaatccct

SEQ ID NO: 78 - DNA, 14 bp, Artificial Sequence
  Primer
atcacattgccagg

SEQ ID NO: 79 - DNA, 30 bp, Artificial Sequence
  Primer
catgatcagctgggccaagatcagaccgag

SEQ ID NO: 80 - DNA, 15 bp, Artificial Sequence
  Primer
cattgcacttgtctc

SEQ ID NO: 81 - DNA, 29 bp, Artificial Sequence
  Primer
catgatcagctgggccaagagcctatcct

SEQ ID NO: 82 - DNA, 17 bp, Artificial Sequence
  Primer
ttcaagtaatccaggat

SEQ ID NO: 83 - DNA, 29 bp, Artificial Sequence
  Primer
catgatcagctgggccaagaaacctatcc

SEQ ID NO: 84 - DNA, 17 bp, Artificial Sequence
  Primer
ttcaagtaattcaggat

SEQ ID NO: 85 - DNA, 30 bp, Artificial Sequence
  Primer
catgatcagctgggccaagagcggaactta

SEQ ID NO: 86 - DNA, 14 bp, Artificial Sequence
  Primer
ttcacagtggctaa

SEQ ID NO: 87 - DNA, 30 bp, Artificial Sequence
  Primer
catgatcagctgggccaagagcagaactta

SEQ ID NO: 88 - DNA, 14 bp, Artificial Sequence
  Primer
ttcacagtggctaa

SEQ ID NO: 89 - DNA, 30 bp, Artificial Sequence
  Primer
catgatcagctgggccaagactcaatagac

SEQ ID NO: 90 - DNA, 14 bp, Artificial Sequence
  Primer
aaggagctcacagt

SEQ ID NO: 91 - DNA, 28 bp, Artificial Sequence
  Primer
catgatcagctgggccaagaaaccgatt

SEQ ID NO: 92 - DNA, 16 bp, Artificial Sequence
  Primer
tagcaccatctgaaat

SEQ ID NO: 93 - DNA, 29 bp, Artificial Sequence
  Primer
catgatcagctgggccaagaaacactgat

SEQ ID NO: 94 - DNA, 19 bp, Artificial Sequence
  Primer
tagcaccatttgaaatcag

SEQ ID NO: 95 - DNA, 30 bp, Artificial Sequence
  Primer
catgatcagctgggccaagacttccagtcg

SEQ ID NO: 96 - DNA, 16 bp, Artificial Sequence
  Primer
tgtaaacatcctcgac

SEQ ID NO: 97 - DNA, 30 bp, Artificial Sequence
  Primer
catgatcagctgggccaagaagctgagtgt

SEQ ID NO: 98 - DNA, 17 bp, Artificial Sequence
  Primer
tgtaaacatcctacact

SEQ ID NO: 99 - DNA, 30 bp, Artificial Sequence
  Primer
catgatcagctgggccaagagctgagagtg

SEQ ID NO: 100 - DNA, 17 bp, Artificial Sequence
  Primer
tgtaaacatcctacact

SEQ ID NO: 101 - DNA, 30 bp, Artificial Sequence; identical to SEQ ID NO: 95
  Primer

SEQ ID NO: 102 - DNA, 14 bp, Artificial Sequence
  Primer
tgtaaacatccccg

SEQ ID NO: 103 - DNA, 29 bp, Artificial Sequence
  Primer
catgatcagctgggccaagagctgtaaac

SEQ ID NO: 104 - DNA, 17 bp, Artificial Sequence
  Primer
ctttcagtcggatgttt

SEQ ID NO: 105 - DNA, 30 bp, Artificial Sequence
  Primer
catgatcagctgggccaagatccagtcaag

SEQ ID NO: 106 - DNA, 16 bp, Artificial Sequence
  Primer
tgtaaacatccttgac

SEQ ID NO: 107 - DNA, 30 bp, Artificial Sequence
  Primer
catgatcagctgggccaagacagctatgcc

SEQ ID NO: 108 - DNA, 14 bp, Artificial Sequence
  Primer
ggcaagatgctggc

SEQ ID NO: 109 - DNA, 30 bp, Artificial Sequence
  Primer
catgatcagctgggccaagagcaacttagt

SEQ ID NO: 110 - DNA, 17 bp, Artificial Sequence
  Primer
tattgcacattactaag

SEQ ID NO: 111 - DNA, 29 bp, Artificial Sequence
  Primer
catgatcagctgggccaagacaatgcaac

SEQ ID NO: 112 - DNA, 15 bp, Artificial Sequence
  Primer
gtgcattgtagttgc

SEQ ID NO: 113 - DNA, 30 bp, Artificial Sequence
  Primer
catgatcagctgggccaagaaacaaccagc

SEQ ID NO: 114 - DNA, 14 bp, Artificial Sequence
  Primer
tggcagtgtcttag

SEQ ID NO: 115 - DNA, 30 bp, Artificial Sequence
  Primer
catgatcagctgggccaagacaatcagcta

SEQ ID NO: 116 - DNA, 14 bp, Artificial Sequence
  Primer
taggcagtgtcatt

SEQ ID NO: 117 - DNA, 30 bp, Artificial Sequence
  Primer
catgatcagctgggccaagagcaatcagct

SEQ ID NO: 118 - DNA, 14 bp, Artificial Sequence
  Primer
aggcagtgtagtta

SEQ ID NO: 119 - DNA, 30 bp, Artificial Sequence
  Primer
catgatcagctgggccaagacaggccggga

SEQ ID NO: 120 - DNA, 15 bp, Artificial Sequence
  Primer
tattgcacttgtccc

SEQ ID NO: 121 - DNA, 30 bp, Artificial Sequence
  Primer
catgatcagctgggccaagactacctgcac

SEQ ID NO: 122 - DNA, 14 bp, Artificial Sequence
  Primer
aaagtgctgttcgt

SEQ ID NO: 123 - DNA, 30 bp, Artificial Sequence
  Primer
catgatcagctgggccaagatgctcaataa

SEQ ID NO: 124 - DNA, 19 bp, Artificial Sequence
  Primer
ttcaacgggtatttattga

SEQ ID NO: 125 - DNA, 30 bp, Artificial Sequence
  Primer
catgatcagctgggccaagagcaaaaatgt

SEQ ID NO: 126 - DNA, 14 bp, Artificial Sequence
  Primer
tttggcactagcac

SEQ ID NO: 127 - DNA, 30 bp, Artificial Sequence
  Primer
catgatcagctgggccaagaaacaatacaa

SEQ ID NO: 128 - DNA, 15 bp, Artificial Sequence
  Primer
tgaggtagtaagttg

SEQ ID NO: 129 - DNA, 30 bp, Artificial Sequence
  Primer
catgatcagctgggccaagacacaagatcg

SEQ ID NO: 130 - DNA, 14 bp, Artificial Sequence
  Primer
aacccgtagatccg

SEQ ID NO: 131 - DNA, 30 bp, Artificial Sequence
  Primer
catgatcagctgggccaagacgcaaggtcg

SEQ ID NO: 132 - DNA, 14 bp, Artificial Sequence
  Primer
cacccgtagaaccg

SEQ ID NO: 133 - DNA, 30 bp, Artificial Sequence
  Primer
catgatcagctgggccaagacacaagttcg

SEQ ID NO: 134 - DNA, 14 bp, Artificial Sequence
  Primer
aacccgtagatccg

SEQ ID NO: 135 - DNA, 30 bp, Artificial Sequence
  Primer
catgatcagctgggccaagacttcagttat

SEQ ID NO: 136 - DNA, 18 bp, Artificial Sequence
  Primer
tacagtactgtgataact

SEQ ID NO: 137 - DNA, 30 bp, Artificial Sequence
  Primer
catgatcagctgggccaagatcatagccct

SEQ ID NO: 138 - DNA, 14 bp, Artificial Sequence
  Primer
agcagcattgtaca

SEQ ID NO: 139 - DNA, 30 bp, Artificial Sequence
  Primer
catgatcagctgggccaagaacaggagtct

SEQ ID NO: 140 - DNA, 15 bp, Artificial Sequence
  Primer
tcaaatgctcagact

SEQ ID NO: 141 - DNA, 30 bp, Artificial Sequence
  Primer
catgatcagctgggccaagagctacctgca

SEQ ID NO: 142 - DNA, 16 bp, Artificial Sequence
  Primer
aaaagtgcttacagtg

SEQ ID NO: 143 - DNA, 30 bp, Artificial Sequence
  Primer
catgatcagctgggccaagaatctgcactg

SEQ ID NO: 144 - DNA, 15 bp, Artificial Sequence
  Primer
taaagtgctgacagt

SEQ ID NO: 145 - DNA, 28 bp, Artificial Sequence
  Primer
catgatcagctgggccaagatgatagcc

SEQ ID NO: 146 - DNA, 15 bp, Artificial Sequence
  Primer
agcagcattgtacag

SEQ ID NO: 147 - DNA, 30 bp, Artificial Sequence
  Primer
catgatcagctgggccaagaacaaacacca

SEQ ID NO: 148 - DNA, 14 bp, Artificial Sequence
  Primer
tggagtgtgacaat

SEQ ID NO: 149 - DNA, 30 bp, Artificial Sequence
  Primer
catgatcagctgggccaagatggcattcac

SEQ ID NO: 150 - DNA, 14 bp, Artificial Sequence
  Primer
ttaaggcacgcggt

SEQ ID NO: 151 - DNA, 30 bp, Artificial Sequence
  Primer
catgatcagctgggccaagacacaggttaa

SEQ ID NO: 152 - DNA, 14 bp, Artificial Sequence
  Primer
tccctgagaccctt

SEQ ID NO: 153 - DNA, 30 bp, Artificial Sequence
  Primer
catgatcagctgggccaagatcacaagtta

SEQ ID NO: 154 - DNA, 14 bp, Artificial Sequence
  Primer
tccctgagacccta

SEQ ID NO: 155 - DNA, 30 bp, Artificial Sequence
  Primer
catgatcagctgggccaagagcattattac

SEQ ID NO: 156 - DNA, 14 bp, Artificial Sequence
  Primer
tcgtaccgtgagta

SEQ ID NO: 157 - DNA, 28 bp, Artificial Sequence
  Primer
catgatcagctgggccaagacgcgtacc

SEQ ID NO: 158 - DNA, 19 bp, Artificial Sequence
  Primer
cattattacttttggtacg

SEQ ID NO: 159 - DNA, 30 bp, Artificial Sequence
  Primer
catgatcagctgggccaagaagccaagctc

SEQ ID NO: 160 - DNA, 14 bp, Artificial Sequence
  Primer
tcggatccgtctga

SEQ ID NO: 161 - DNA, 30 bp, Artificial Sequence
  Primer
catgatcagctgggccaagaaaaagagacc

SEQ ID NO: 162 - DNA, 14 bp, Artificial Sequence
  Primer
tcacagtgaaccgg

SEQ ID NO: 163 - DNA, 30 bp, Artificial Sequence
  Primer
catgatcagctgggccaagagaaagagacc

SEQ ID NO: 164 - DNA, 14 bp, Artificial Sequence
  Primer
tcacagtgaaccgg

SEQ ID NO: 165 - DNA, 30 bp, Artificial Sequence
  Primer
catgatcagctgggccaagagcaagcccag

SEQ ID NO: 166 - DNA, 14 bp, Artificial Sequence
  Primer
ctttttgcggtctg

SEQ ID NO: 167 - DNA, 30 bp, Artificial Sequence
  Primer
catgatcagctgggccaagaatgccctttt

SEQ ID NO: 168 - DNA, 17 bp, Artificial Sequence
  Primer
cagtgcaatgttaaaag

SEQ ID NO: 169 - DNA, 30 bp, Artificial Sequence
  Primer
catgatcagctgggccaagaatgccctttc

SEQ ID NO: 170 - DNA, 14 bp, Artificial Sequence
  Primer
cagtgcaatgatga

SEQ ID NO: 171 - DNA, 30 bp, Artificial Sequence
  Primer
catgatcagctgggccaagacgaccatggc

SEQ ID NO: 172 - DNA, 15 bp, Artificial Sequence
  Primer
taacagtctacagcc

SEQ ID NO: 173 - DNA, 30 bp, Artificial Sequence
  Primer
catgatcagctgggccaagaacagctggtt

SEQ ID NO: 174 - DNA, 14 bp, Artificial Sequence
  Primer
ttggtccccttcaa

SEQ ID NO: 175 - DNA, 30 bp, Artificial Sequence
  Primer
catgatcagctgggccaagatagctggttg

SEQ ID NO: 176 - DNA, 14 bp, Artificial Sequence
  Primer
ttggtccccttcaa

SEQ ID NO: 177 - DNA, 30 bp, Artificial Sequence
  Primer
catgatcagctgggccaagaccctctggtc

SEQ ID NO: 178 - DNA, 14 bp, Artificial Sequence
  Primer
tgtgactggttgac

SEQ ID NO: 179 - DNA, 30 bp, Artificial Sequence
  Primer
catgatcagctgggccaagatcacatagga

SEQ ID NO: 180 - DNA, 17 bp, Artificial Sequence
  Primer
tatggctttttattcct

SEQ ID NO: 181 - DNA, 30 bp, Artificial Sequence
  Primer
catgatcagctgggccaagacacataggaa

SEQ ID NO: 182 - DNA, 16 bp, Artificial Sequence
  Primer
tatggcttttcattcc

SEQ ID NO: 183 - DNA, 30 bp, Artificial Sequence
  Primer
catgatcagctgggccaagatccatcatca

SEQ ID NO: 184 - DNA, 18 bp, Artificial Sequence
  Primer
actccatttgttttgatg

SEQ ID NO: 185 - DNA, 30 bp, Artificial Sequence
  Primer
catgatcagctgggccaagactacgcgtat

SEQ ID NO: 186 - DNA, 18 bp, Artificial Sequence
  Primer
tattgcttaagaatacgc

SEQ ID NO: 187 - DNA, 29 bp, Artificial Sequence
  Primer
catgatcagctgggccaagacggcctgat

SEQ ID NO: 188 - DNA, 14 bp, Artificial Sequence
  Primer
agctggtgttgtga

SEQ ID NO: 189 - DNA, 30 bp, Artificial Sequence
  Primer
catgatcagctgggccaagaagacacgtgc

SEQ ID NO: 190 - DNA, 14 bp, Artificial Sequence
  Primer
tctacagtgcacgt

SEQ ID NO: 191 - DNA, 30 bp, Artificial Sequence
  Primer
catgatcagctgggccaagactaccatagg

SEQ ID NO: 192 - DNA, 14 bp, Artificial Sequence
  Primer
agtggttttaccct

SEQ ID NO: 193 - DNA, 29 bp, Artificial Sequence
  Primer
catgatcagctgggccaagaccatcttta

SEQ ID NO: 194 - DNA, 16 bp, Artificial Sequence
  Primer
taacactgtctggtaa

SEQ ID NO: 195 - DNA, 28 bp, Artificial Sequence
  Primer
catgatcagctgggccaagatccataaa

SEQ ID NO: 196 - DNA, 16 bp, Artificial Sequence
  Primer
tgtagtgtttcctact

SEQ ID NO: 197 - DNA, 28 bp, Artificial Sequence
  Primer
catgatcagctgggccaagatgagctac

SEQ ID NO: 198 - DNA, 15 bp, Artificial Sequence
  Primer
tgagatgaagcactg

SEQ ID NO: 199 - DNA, 29 bp, Artificial Sequence
  Primer
catgatcagctgggccaagactagtacat

SEQ ID NO: 200 - DNA, 16 bp, Artificial Sequence
  Primer
tacagtatagatgatg

SEQ ID NO: 201 - DNA, 29 bp, Artificial Sequence
  Primer
catgatcagctgggccaagaaagggattc

SEQ ID NO: 202 - DNA, 14 bp, Artificial Sequence
  Primer
gtccagttttccca

SEQ ID NO: 203 - DNA, 28 bp, Artificial Sequence
  Primer
catgatcagctgggccaagaaacccatg

SEQ ID NO: 204 - DNA, 16 bp, Artificial Sequence
  Primer
tgagaactgaattcca

SEQ ID NO: 205 - DNA, 30 bp, Artificial Sequence
  Primer
catgatcagctgggccaagagcagaagcat

SEQ ID NO: 206 - DNA, 14 bp, Artificial Sequence
  Primer
gtgtgtggaaatgc

SEQ ID NO: 207 - DNA, 29 bp, Artificial Sequence
  Primer
catgatcagctgggccaagaacaaagttc

SEQ ID NO: 208 - DNA, 18 bp, Artificial Sequence
  Primer
tcagtgcactacagaact

SEQ ID NO: 209 - DNA, 29 bp, Artificial Sequence
  Primer
catgatcagctgggccaagaacaaagttc

SEQ ID NO: 210 - DNA, 14 bp, Artificial Sequence
  Primer
tcagtgcatcacag

SEQ ID NO: 211 - DNA, 29 bp, Artificial Sequence
  Primer
catgatcagctgggccaagaggagtgaag

SEQ ID NO: 212 - DNA, 14 bp, Artificial Sequence
  Primer
tctggctccgtgtc

SEQ ID NO: 213 - DNA, 28 bp, Artificial Sequence
  Primer
catgatcagctgggccaagacactggta

SEQ ID NO: 214 - DNA, 14 bp, Artificial Sequence
  Primer
tctcccaacccttg

SEQ ID NO: 215 - DNA, 29 bp, Artificial Sequence
  Primer
catgatcagctgggccaagacctcaagga

SEQ ID NO: 216 - DNA, 15 bp, Artificial Sequence
  Primer
actagactgaagctc

SEQ ID NO: 217 - DNA, 29 bp, Artificial Sequence
  Primer
catgatcagctgggccaagacccaagttc

SEQ ID NO: 218 - DNA, 14 bp, Artificial Sequence
  Primer
tcagtgcatgacag

SEQ ID NO: 219 - DNA, 29 bp, Artificial Sequence
  Primer
catgatcagctgggccaagatcacttttg

SEQ ID NO: 220 - DNA, 16 bp, Artificial Sequence
  Primer
ttgcatagtcacaaaa

SEQ ID NO: 221 - DNA, 29 bp, Artificial Sequence
  Primer
catgatcagctgggccaagaaataggtca

SEQ ID NO: 222 - DNA, 17 bp, Artificial Sequence
  Primer
aatcatacacggttgac

SEQ ID NO: 223 - DNA, 29 bp, Artificial Sequence
  Primer
catgatcagctgggccaagacgaaggcaa

SEQ ID NO: 224 - DNA, 15 bp, Artificial Sequence
  Primer
taggttatccgtgtt

SEQ ID NO: 225 - DNA, 28 bp, Artificial Sequence
  Primer
catgatcagctgggccaagacccctatc

SEQ ID NO: 226 - DNA, 20 bp, Artificial Sequence
  Primer
ttaatgctaatcgtgatagg

SEQ ID NO: 227 - DNA, 29 bp, Artificial Sequence
  Primer
catgatcagctgggccaagaactcaccga

SEQ ID NO: 228 - DNA, 16 bp, Artificial Sequence
  Primer
aacattcaacgctgtc

SEQ ID NO: 229 - DNA, 29 bp, Artificial Sequence
  Primer
catgatcagctgggccaagaactcaccga

SEQ ID NO: 230 - DNA, 16 bp, Artificial Sequence
  Primer
aacattcaacctgtcg

SEQ ID NO: 231 - DNA, 30 bp, Artificial Sequence
  Primer
catgatcagctgggccaagatagttggcaa

SEQ ID NO: 232 - DNA, 15 bp, Artificial Sequence
  Primer
tggttctagacttgc

SEQ ID NO: 233 - DNA, 29 bp, Artificial Sequence
  Primer
catgatcagctgggccaagatgtgagttc

SEQ ID NO: 234 - DNA, 14 bp, Artificial Sequence
  Primer
tttggcaatggtag

SEQ ID NO: 235 - DNA, 29 bp, Artificial Sequence
  Primer
catgatcagctgggccaagacagtgaatt

SEQ ID NO: 236 - DNA, 14 bp, Artificial Sequence
  Primer
tatggcactggtag

SEQ ID NO: 237 - DNA, 29 bp, Artificial Sequence
  Primer
catgatcagctgggccaagaacccttatc

SEQ ID NO: 238 - DNA, 14 bp, Artificial Sequence
  Primer
tggacggagaactg

SEQ ID NO: 239 - DNA, 29 bp, Artificial Sequence
  Primer
catgatcagctgggccaagaaagcccaaa

SEQ ID NO: 240 - DNA, 19 bp, Artificial Sequence
  Primer
caaagaattctccttttgg

SEQ ID NO: 241 - DNA, 30 bp, Artificial Sequence
  Primer
catgatcagctgggccaagacggctgcaac

SEQ ID NO: 242 - DNA, 14 bp, Artificial Sequence
  Primer
tcgtgtcttgtgtt

SEQ ID NO: 243 - DNA, 30 bp, Artificial Sequence
  Primer
catgatcagctgggccaagaaccctccacc

SEQ ID NO: 244 - DNA, 14 bp, Artificial Sequence
  Primer
catcccttgcatgg

SEQ ID NO: 245 - DNA, 29 bp, Artificial Sequence
  Primer
catgatcagctgggccaagaactgatatc

SEQ ID NO: 246 - DNA, 14 bp, Artificial Sequence
  Primer
gtgcctactgagct

SEQ ID NO: 247 - DNA, 29 bp, Artificial Sequence
  Primer
catgatcagctgggccaagaacctaatat

SEQ ID NO: 248 - DNA, 20 bp, Artificial Sequence
  Primer
tgatatgtttgatatattag

SEQ ID NO: 249 - DNA, 29 bp, Artificial Sequence
  Primer
catgatcagctgggccaagaagctgcttt

SEQ ID NO: 250 - DNA, 17 bp, Artificial Sequence
  Primer
caacggaatcccaaaag

SEQ ID NO: 251 - DNA, 29 bp, Artificial Sequence
  Primer
catgatcagctgggccaagaggctgtcaa

SEQ ID NO: 252 - DNA, 17 bp, Artificial Sequence
  Primer
ctgacctatgaattgac

SEQ ID NO: 253 - DNA, 29 bp, Artificial Sequence
  Primer
catgatcagctgggccaagactgggactt

SEQ ID NO: 254 - DNA, 15 bp, Artificial Sequence
  Primer
aactggcctacaaag

SEQ ID NO: 255 - DNA, 28 bp, Artificial Sequence
  Primer
catgatcagctgggccaagatccacatg

SEQ ID NO: 256 - DNA, 16 bp, Artificial Sequence
  Primer
tgtaacagcaactcca

SEQ ID NO: 257 - DNA, 29 bp, Artificial Sequence
  Primer
catgatcagctgggccaagagccaatatt

SEQ ID NO: 258 - DNA, 16 bp, Artificial Sequence
  Primer
tagcagcacagaaata

SEQ ID NO: 259 - DNA, 30 bp, Artificial Sequence
  Primer
catgatcagctgggccaagaccaacaacag

SEQ ID NO: 260 - DNA, 15 bp, Artificial Sequence
  Primer
taggtagtttcctgt

SEQ ID NO: 261 - DNA, 30 bp, Artificial Sequence
  Primer
catgatcagctgggccaagaccaacaacat

SEQ ID NO: 262 - DNA, 17 bp, Artificial Sequence
  Primer
taggtagtttcatgttg

SEQ ID NO: 263 - DNA, 29 bp, Artificial Sequence
  Primer
catgatcagctgggccaagagctgggtgg

SEQ ID NO: 264 - DNA, 14 bp, Artificial Sequence
  Primer
ttcaccaccttctc

SEQ ID NO: 265 - DNA, 28 bp, Artificial Sequence
  Primer
catgatcagctgggccaagacctatctc

SEQ ID NO: 266 - DNA, 14 bp, Artificial Sequence
  Primer
ggtccagaggggag

SEQ ID NO: 267 - DNA, 29 bp, Artificial Sequence
  Primer
catgatcagctgggccaagaaaccaatgt

SEQ ID NO: 268 - DNA, 15 bp, Artificial Sequence
  Primer
tacagtagtctgcac

SEQ ID NO: 269 - DNA, 29 bp, Artificial Sequence
  Primer
catgatcagctgggccaagagaacaggta

SEQ ID NO: 270 - DNA, 14 bp, Artificial Sequence
  Primer
cccagtgttcagac

SEQ ID NO: 271 - DNA, 30 bp, Artificial Sequence
  Primer
catgatcagctgggccaagagaacagatag

SEQ ID NO: 272 - DNA, 14 bp, Artificial Sequence
  Primer
cccagtgtttagac

SEQ ID NO: 273 - DNA, 29 bp, Artificial Sequence
  Primer
catgatcagctgggccaagaacatcgtta

SEQ ID NO: 274 - DNA, 14 bp, Artificial Sequence
  Primer
taacactgtctggt

SEQ ID NO: 275 - DNA, 29 bp, Artificial Sequence
  Primer
catgatcagctgggccaagagtcatcatt

SEQ ID NO: 276 - DNA, 17 bp, Artificial Sequence
  Primer
taatactgcctggtaat

SEQ ID NO: 277 - DNA, 30 bp, Artificial Sequence
  Primer
catgatcagctgggccaagattttcccatg

SEQ ID NO: 278 - DNA, 15 bp, Artificial Sequence
  Primer
agaggtatagggcat

SEQ ID NO: 279 - DNA, 29 bp, Artificial Sequence
  Primer
catgatcagctgggccaagactagtggtc

SEQ ID NO: 280 - DNA, 17 bp, Artificial Sequence
  Primer
gtgaaatgtttaggacc

SEQ ID NO: 281 - DNA, 29 bp, Artificial Sequence
  Primer
catgatcagctgggccaagaaggcatagg

SEQ ID NO: 282 - DNA, 15 bp, Artificial Sequence
  Primer
ttccctttgtcatcc

SEQ ID NO: 283 - DNA, 30 bp, Artificial Sequence
  Primer
catgatcagctgggccaagacagactccgg

SEQ ID NO: 284 - DNA, 14 bp, Artificial Sequence
  Primer
tccttcattccacc

SEQ ID NO: 285 - DNA, 27 bp, Artificial Sequence
  Primer
catgatcagctgggccaagaccacaca

SEQ ID NO: 286 - DNA, 18 bp, Artificial Sequence
  Primer
tggaatgtaaggaagtgt

SEQ ID NO: 287 - DNA, 33 bp, Artificial Sequence
  Primer
catgatcagctgggccaagaacaagctttttgc

SEQ ID NO: 288 - DNA, 17 bp, Artificial Sequence
  Primer
ataagacgagcaaaaag

SEQ ID NO: 289 - DNA, 30 bp, Artificial Sequence
  Primer
catgatcagctgggccaagatcagccgctg

SEQ ID NO: 290 - DNA, 14 bp, Artificial Sequence
  Primer
ctgtgcgtgtgaca

SEQ ID NO: 291 - DNA, 29 bp, Artificial Sequence
  Primer
catgatcagctgggccaagaaggcgaagg

SEQ ID NO: 292 - DNA, 15 bp, Artificial Sequence
  Primer
ttccctttgtcatcc

SEQ ID NO: 293 - DNA, 29 bp, Artificial Sequence
  Primer
catgatcagctgggccaagaggccgtgac

SEQ ID NO: 294 - DNA, 16 bp, Artificial Sequence
  Primer
taacagtctccagtca

SEQ ID NO: 295 - DNA, 30 bp, Artificial Sequence
  Primer
catgatcagctgggccaagaggtacaatca

SEQ ID NO: 296 - DNA, 14 bp, Artificial Sequence
  Primer
accatcgaccgttg

SEQ ID NO: 297 - DNA, 30 bp, Artificial Sequence
  Primer
catgatcagctgggccaagactgcctgtct

SEQ ID NO: 298 - DNA, 14 bp, Artificial Sequence
  Primer
acagcaggcacaga

SEQ ID NO: 299 - DNA, 29 bp, Artificial Sequence
  Primer
catgatcagctgggccaagagtctgtcaa

SEQ ID NO: 300 - DNA, 17 bp, Artificial Sequence
  Primer
atgacctatgaattgac

SEQ ID NO: 301 - DNA, 29 bp, Artificial Sequence
  Primer
catgatcagctgggccaagacacagttgc

SEQ ID NO: 302 - DNA, 15 bp, Artificial Sequence
  Primer
taatctcagctggca

SEQ ID NO: 303 - DNA, 29 bp, Artificial Sequence
  Primer
catgatcagctgggccaagaatccaatca

SEQ ID NO: 304 - DNA, 18 bp, Artificial Sequence
  Primer
tactgcatcaggaactga

SEQ ID NO: 305 - DNA, 29 bp, Artificial Sequence
  Primer
catgatcagctgggccaagaacatggtta

SEQ ID NO: 306 - DNA, 16 bp, Artificial Sequence
  Primer
ttgtgcttgatctaac

SEQ ID NO: 307 - DNA, 30 bp, Artificial Sequence
  Primer
catgatcagctgggccaagaaaagtgtcag

SEQ ID NO: 308 - DNA, 14 bp, Artificial Sequence
  Primer
ccacaccgtatctg

SEQ ID NO: 309 - DNA, 29 bp, Artificial Sequence
  Primer
catgatcagctgggccaagagaaacccag

SEQ ID NO: 310 - DNA, 15 bp, Artificial Sequence
  Primer
agctacattgtctgc

SEQ ID NO: 311 - DNA, 28 bp, Artificial Sequence
  Primer
catgatcagctgggccaagagagaccca

SEQ ID NO: 312 - DNA, 14 bp, Artificial Sequence
  Primer
agctacatctggct

SEQ ID NO: 313 - DNA, 30 bp, Artificial Sequence
  Primer
catgatcagctgggccaagaggggtatttg

SEQ ID NO: 314 - DNA, 15 bp, Artificial Sequence
  Primer
tgtcagtttgtcaaa

SEQ ID NO: 315 - DNA, 28 bp, Artificial Sequence
  Primer
catgatcagctgggccaagataaacgga

SEQ ID NO: 316 - DNA, 16 bp, Artificial Sequence
  Primer
caagtcactagtggtt

SEQ ID NO: 317 - DNA, 29 bp, Artificial Sequence
  Primer
catgatcagctgggccaagaacaggattg

SEQ ID NO: 318 - DNA, 15 bp, Artificial Sequence
  Primer
agggccccccctcaa

SEQ ID NO: 319 - DNA, 29 bp, Artificial Sequence
  Primer
catgatcagctgggccaagaatgtatgtg

SEQ ID NO: 320 - DNA, 14 bp, Artificial Sequence
  Primer
tggtttaccgtccc

SEQ ID NO: 321 - DNA, 30 bp, Artificial Sequence
  Primer
catgatcagctgggccaagagctttgacaa

SEQ ID NO: 322 - DNA, 17 bp, Artificial Sequence
  Primer
cagtgcaatagtattgt

SEQ ID NO: 323 - DNA, 30 bp, Artificial Sequence
  Primer
catgatcagctgggccaagaaaagcaagta

SEQ ID NO: 324 - DNA, 15 bp, Artificial Sequence
  Primer
taaacgtggatgtac

SEQ ID NO: 325 - DNA, 30 bp, Artificial Sequence
  Primer
catgatcagctgggccaagatcaccaaaac

SEQ ID NO: 326 - DNA, 15 bp, Artificial Sequence
  Primer
taagtgcttccatgt

SEQ ID NO: 327 - DNA, 30 bp, Artificial Sequence
  Primer
catgatcagctgggccaagaagaaagcact

SEQ ID NO: 328 - DNA, 17 bp, Artificial Sequence
  Primer
actttaacatggaagtg

SEQ ID NO: 329 - DNA, 30 bp, Artificial Sequence
  Primer
catgatcagctgggccaagactactaaaac

SEQ ID NO: 330 - DNA, 15 bp, Artificial Sequence
  Primer
taagtgcttccatgt

SEQ ID NO: 331 - DNA, 30 bp, Artificial Sequence
  Primer
catgatcagctgggccaagaacactcaaac

SEQ ID NO: 332 - DNA, 15 bp, Artificial Sequence
  Primer
taagtgcttccatgt

SEQ ID NO: 333 - DNA, 29 bp, Artificial Sequence
  Primer
catgatcagctgggccaagacagcaggta

SEQ ID NO: 334 - DNA, 17 bp, Artificial Sequence
  Primer
tttaacatgggggtacc

SEQ ID NO: 335 - DNA, 29 bp, Artificial Sequence
  Primer
catgatcagctgggccaagaccactgaaa

SEQ ID NO: 336 - DNA, 19 bp, Artificial Sequence
  Primer
taagtgcttccatgtttca

SEQ ID NO: 337 - DNA, 28 bp, Artificial Sequence
  Primer
catgatcagctgggccaagattcgccct

SEQ ID NO: 338 - DNA, 18 bp, Artificial Sequence
  Primer
aaaagctgggttgagagg

SEQ ID NO: 339 - DNA, 30 bp, Artificial Sequence
  Primer
catgatcagctgggccaagaagaggtcgac

SEQ ID NO: 340 - DNA, 14 bp, Artificial Sequence
  Primer
gcacattacacggt

SEQ ID NO: 341 - DNA, 30 bp, Artificial Sequence
  Primer
catgatcagctgggccaagaccagcagcac

SEQ ID NO: 342 - DNA, 14 bp, Artificial Sequence
  Primer
ccactgccccaggt

SEQ ID NO: 343 - DNA, 30 bp, Artificial Sequence
  Primer
catgatcagctgggccaagaacaccaatgc

SEQ ID NO: 344 - DNA, 14 bp, Artificial Sequence
  Primer
cgcatcccctaggg

SEQ ID NO: 345 - DNA, 30 bp, Artificial Sequence
  Primer
catgatcagctgggccaagaacacttactg

SEQ ID NO: 346 - DNA, 14 bp, Artificial Sequence
  Primer
cctagtaggtgtcc

SEQ ID NO: 347 - DNA, 30 bp, Artificial Sequence
  Primer
catgatcagctgggccaagactggaggaag

SEQ ID NO: 348 - DNA, 14 bp, Artificial Sequence
  Primer
cctctgggcccttc

SEQ ID NO: 349 - DNA, 30 bp, Artificial Sequence
  Primer
catgatcagctgggccaagaacggaagggc

SEQ ID NO: 350 - DNA, 14 bp, Artificial Sequence
  Primer
ctggccctctctgc

SEQ ID NO: 351 - DNA, 30 bp, Artificial Sequence
  Primer
catgatcagctgggccaagatctctgcagg

SEQ ID NO: 352 - DNA, 14 bp, Artificial Sequence
  Primer
gcaaagcacacggc

SEQ ID NO: 353 - DNA, 30 bp, Artificial Sequence
  Primer
catgatcagctgggccaagattctaggata

SEQ ID NO: 354 - DNA, 14 bp, Artificial Sequence
  Primer
gcccctgggcctat

SEQ ID NO: 355 - DNA, 30 bp, Artificial Sequence
  Primer
catgatcagctgggccaagaaaaggcatca

SEQ ID NO: 356 - DNA, 15 bp, Artificial Sequence
  Primer
tccagctcctatatg

SEQ ID NO: 357 - DNA, 30 bp, Artificial Sequence
  Primer
catgatcagctgggccaagatcaacaaaat

SEQ ID NO: 358 - DNA, 17 bp, Artificial Sequence
  Primer
tccagcatcagtgattt

SEQ ID NO: 359 - DNA, 29 bp, Artificial Sequence
  Primer
catgatcagctgggccaagatgagctcct

SEQ ID NO: 360 - DNA, 15 bp, Artificial Sequence
  Primer
tccctgtcctccagg

SEQ ID NO: 361 - DNA, 30 bp, Artificial Sequence
  Primer
catgatcagctgggccaagaggctataaag

SEQ ID NO: 362 - DNA, 14 bp, Artificial Sequence
  Primer
tccgtctcagttac

SEQ ID NO: 363 - DNA, 28 bp, Artificial Sequence
  Primer
catgatcagctgggccaagagacgggtg

SEQ ID NO: 364 - DNA, 16 bp, Artificial Sequence
  Primer
tctcacacagaaatcg

SEQ ID NO: 365 - DNA, 30 bp, Artificial Sequence
  Primer
catgatcagctgggccaagagccctggact

SEQ ID NO: 366 - DNA, 14 bp, Artificial Sequence
  Primer
tgctgactcctagt

SEQ ID NO: 367 - DNA, 30 bp, Artificial Sequence
  Primer
catgatcagctgggccaagaagaggcaggc

SEQ ID NO: 368 - DNA, 14 bp, Artificial Sequence
  Primer
tgtctgcccgcatg

SEQ ID NO: 369 - DNA, 30 bp, Artificial Sequence
  Primer
catgatcagctgggccaagatacagatgga

SEQ ID NO: 370 - DNA, 15 bp, Artificial Sequence
  Primer
aattgcacggtatcc

SEQ ID NO: 371 - DNA, 30 bp, Artificial Sequence
  Primer
catgatcagctgggccaagatcaccattgc

SEQ ID NO: 372 - DNA, 17 bp, Artificial Sequence
  Primer
aattgcactttagcaat

SEQ ID NO: 373 - DNA, 30 bp, Artificial Sequence
  Primer
catgatcagctgggccaagaaaacgtggaa

SEQ ID NO: 374 - DNA, 18 bp, Artificial Sequence
  Primer
acatagaggaaattccac

SEQ ID NO: 375 - DNA, 30 bp, Artificial Sequence
  Primer
catgatcagctgggccaagaccaggttcca

SEQ ID NO: 376 - DNA, 14 bp, Artificial Sequence
  Primer
gcctgctggggtgg

SEQ ID NO: 377 - DNA, 30 bp, Artificial Sequence
  Primer
catgatcagctgggccaagaacactcaaaa

SEQ ID NO: 378 - DNA, 14 bp, Artificial Sequence
  Primer
gtgccgccatcttt

SEQ ID NO: 379 - DNA, 30 bp, Artificial Sequence
  Primer
catgatcagctgggccaagaacgctcaaat

SEQ ID NO: 380 - DNA, 14 bp, Artificial Sequence
  Primer
aaagtgctgcgaca

SEQ ID NO: 381 - DNA, 30 bp, Artificial Sequence
  Primer
catgatcagctgggccaagaggaaagcgcc

SEQ ID NO: 382 - DNA, 14 bp, Artificial Sequence
  Primer
actcaaaatggggg

SEQ ID NO: 383 - DNA, 30 bp, Artificial Sequence
  Primer
catgatcagctgggccaagaacaccccaaa

SEQ ID NO: 384 - DNA, 18 bp, Artificial Sequence
  Primer
gaagtgcttcgattttgg

SEQ ID NO: 385 - DNA, 29 bp, Artificial Sequence
  Primer
catgatcagctgggccaagacacttatca

SEQ ID NO: 386 - DNA, 20 bp, Artificial Sequence
  Primer
ttataatacaacctgataag

SEQ ID NO: 387 - DNA, 30 bp, Artificial Sequence
  Primer
catgatcagctgggccaagatcacgcgagc

SEQ ID NO: 388 - DNA, 14 bp, Artificial Sequence
  Primer
tttgttcgttcggc

SEQ ID NO: 389 - DNA, 28 bp, Artificial Sequence
  Primer
catgatcagctgggccaagaaacatgga

SEQ ID NO: 390 - DNA, 18 bp, Artificial Sequence
  Primer
atcatagaggaaaatcca

SEQ ID NO: 391 - DNA, 30 bp, Artificial Sequence
  Primer
catgatcagctgggccaagaacacaggacc

SEQ ID NO: 392 - DNA, 14 bp, Artificial Sequence
  Primer
ctcctgactccagg

SEQ ID NO: 393 - DNA, 27 bp, Artificial Sequence
  Primer
catgatcagctgggccaagatacgttc

SEQ ID NO: 394 - DNA, 17 bp, Artificial Sequence
  Primer
tggtagactatggaacg

SEQ ID NO: 395 - DNA, 30 bp, Artificial Sequence
  Primer
catgatcagctgggccaagagcgcatgttc

SEQ ID NO: 396 - DNA, 14 bp, Artificial Sequence
  Primer
tggttgaccataga

SEQ ID NO: 397 - DNA, 30 bp, Artificial Sequence
  Primer
catgatcagctgggccaagaaagatgtgga

SEQ ID NO: 398 - DNA, 18 bp, Artificial Sequence
  Primer
tatgtaatatggtccaca

SEQ ID NO: 399 - DNA, 29 bp, Artificial Sequence
  Primer
catgatcagctgggccaagaacagagagc

SEQ ID NO: 400 - DNA, 16 bp, Artificial Sequence
  Primer
tatacaagggcaagct

SEQ ID NO: 401 - DNA, 30 bp, Artificial Sequence
  Primer
catgatcagctgggccaagacgaatccacc

SEQ ID NO: 402 - DNA, 15 bp, Artificial Sequence
  Primer
gaagttgttcgtggt

SEQ ID NO: 403 - DNA, 30 bp, Artificial Sequence
  Primer
catgatcagctgggccaagaagccacaatc

SEQ ID NO: 404 - DNA, 18 bp, Artificial Sequence
  Primer
agatcagaaggtgattgt

SEQ ID NO: 405 - DNA, 29 bp, Artificial Sequence
  Primer
catgatcagctgggccaagaacaggccat

SEQ ID NO: 406 - DNA, 17 bp, Artificial Sequence
  Primer
aatataacacagatggc

SEQ ID NO: 407 - DNA, 30 bp, Artificial Sequence
  Primer
catgatcagctgggccaagaacggctagtg

SEQ ID NO: 408 - DNA, 18 bp, Artificial Sequence
  Primer
acttcacctggtccacta

SEQ ID NO: 409 - DNA, 30 bp, Artificial Sequence
  Primer
catgatcagctgggccaagaggccttctga

SEQ ID NO: 410 - DNA, 14 bp, Artificial Sequence
  Primer
ctggacttagggtc

SEQ ID NO: 411 - DNA, 30 bp, Artificial Sequence; identical to SEQ ID NO: 409
  Primer

SEQ ID NO: 412 - DNA, 14 bp, Artificial Sequence
  Primer
ctggacttggagtc

SEQ ID NO: 413 - DNA, 30 bp, Artificial Sequence
  Primer
catgatcagctgggccaagactgaggggcc

SEQ ID NO: 414 - DNA, 14 bp, Artificial Sequence
  Primer
agctcggtctgagg

SEQ ID NO: 415 - DNA, 30 bp, Artificial Sequence
  Primer
catgatcagctgggccaagattcaaaacat

SEQ ID NO: 416 - DNA, 19 bp, Artificial Sequence
  Primer
cagcagcaattcatgtttt

SEQ ID NO: 417 - DNA, 30 bp, Artificial Sequence
  Primer
catgatcagctgggccaagaggcggacacg

SEQ ID NO: 418 - DNA, 14 bp, Artificial Sequence
  Primer
atcgggaatgtcgt

SEQ ID NO: 419 - DNA, 31 bp, Artificial Sequence
  Primer
catgatcagctgggccaagaacggttttacc

SEQ ID NO: 420 - DNA, 18 bp, Artificial Sequence
  Primer
taatactgtctggtaaaa

SEQ ID NO: 421 - DNA, 30 bp, Artificial Sequence
  Primer
catgatcagctgggccaagatgcatgacgg

SEQ ID NO: 422 - DNA, 14 bp, Artificial Sequence
  Primer
tgtcttgcaggccg

SEQ ID NO: 423 - DNA, 30 bp, Artificial Sequence
  Primer
catgatcagctgggccaagaatgggacatc

SEQ ID NO: 424 - DNA, 16 bp, Artificial Sequence
  Primer
ttgcatatgtaggatg

SEQ ID NO: 425 - DNA, 30 bp, Artificial Sequence
  Primer
catgatcagctgggccaagaaccagctaac

SEQ ID NO: 426 - DNA, 18 bp, Artificial Sequence
  Primer
tggcagtgtattgttagc

SEQ ID NO: 427 - DNA, 30 bp, Artificial Sequence
  Primer
catgatcagctgggccaagatattaggaac

SEQ ID NO: 428 - DNA, 15 bp, Artificial Sequence
  Primer
tttttgcgatgtgtt

SEQ ID NO: 429 - DNA, 30 bp, Artificial Sequence
  Primer
catgatcagctgggccaagaaaactcagta

SEQ ID NO: 430 - DNA, 19 bp, Artificial Sequence
  Primer
aaaccgttaccattactga

SEQ ID NO: 431 - DNA, 28 bp, Artificial Sequence
  Primer
catgatcagctgggccaagaaactatac

SEQ ID NO: 432 - DNA, 15 bp, Artificial Sequence
  Primer
tgaggtagtaggttg

SEQ ID NO: 433 - DNA, 28 bp, Artificial Sequence
  Primer
catgatcagctgggccaagaaaccacac

SEQ ID NO: 434 - DNA, 28 bp, Artificial Sequence
  Primer
catgatcagctgggccaagaaaccatac

SEQ ID NO: 435 - DNA, 28 bp, Artificial Sequence
  Primer
catgatcagctgggccaagaactatgca

SEQ ID NO: 436 - DNA, 15 bp, Artificial Sequence
  Primer
agaggtagtaggttg

SEQ ID NO: 437 - DNA, 28 bp, Artificial Sequence
  Primer
catgatcagctgggccaagaactataca

SEQ ID NO: 438 - DNA, 15 bp, Artificial Sequence
  Primer
tgaggtaggaggttg

SEQ ID NO: 439 - DNA, 28 bp, Artificial Sequence
  Primer
catgatcagctgggccaagaaactatac

SEQ ID NO: 440 - DNA, 15 bp, Artificial Sequence
  Primer
tgaggtagtagattg

SEQ ID NO: 441 - DNA, 28 bp, Artificial Sequence
  Primer
catgatcagctgggccaagaactgtaca

SEQ ID NO: 442 - DNA, 15 bp, Artificial Sequence
  Primer
tgaggtagtagtttg

SEQ ID NO: 443 - DNA, 28 bp, Artificial Sequence
  Primer
catgatcagctgggccaagaacagcaca

SEQ ID NO: 444 - DNA, 15 bp, Artificial Sequence
  Primer
tgaggtagtagtttg

SEQ ID NO: 445 - DNA, 30 bp, Artificial Sequence
  Primer
catgatcagctgggccaagaacaaaagttg

SEQ ID NO: 446 - DNA, 16 bp, Artificial Sequence
  Primer
atcacacaaaggcaac

SEQ ID NO: 447 - DNA, 27 bp, Artificial Sequence
  Primer
catgatcagctgggccaagaacgtgga

SEQ ID NO: 448 - DNA, 17 bp, Artificial Sequence
  Primer
atcatagaggaaaatcc

SEQ ID NO: 449 - DNA, 30 bp, Artificial Sequence
  Primer
catgatcagctgggccaagaacagttcttc

SEQ ID NO: 450 - DNA, 14 bp, Artificial Sequence
  Primer
aagctgccagttga

SEQ ID NO: 451 - DNA, 29 bp, Artificial Sequence
  Primer
catgatcagctgggccaagaccatcatta

SEQ ID NO: 452 - DNA, 14 bp, Artificial Sequence
  Primer
taatactgccgggt

SEQ ID NO: 453 - DNA, 30 bp, Artificial Sequence
  Primer
catgatcagctgggccaagactgttcctgc

SEQ ID NO: 454 - DNA, 14 bp, Artificial Sequence
  Primer
tggctcagttcagc

SEQ ID NO: 455 - DNA, 30 bp, Artificial Sequence
  Primer
catgatcagctgggccaagaaccgatttca

SEQ ID NO: 456 - DNA, 16 bp, Artificial Sequence
  Primer
tagcaccatttgaaat

SEQ ID NO: 457 - DNA, 30 bp, Artificial Sequence
  Primer
catgatcagctgggccaagatatctgcact

SEQ ID NO: 458 - DNA, 15 bp, Artificial Sequence
  Primer
taaggtgcatctagt

SEQ ID NO: 459 - DNA, 30 bp, Artificial Sequence
  Primer
catgatcagctgggccaagagaactgcctt

SEQ ID NO: 460 - DNA, 14 bp, Artificial Sequence
  Primer
tggagagaaaggca

SEQ ID NO: 461 - DNA, 28 bp, Artificial Sequence
  Primer
catgatcagctgggccaagacccaccga

SEQ ID NO: 462 - DNA, 16 bp, Artificial Sequence
  Primer
aacattcattgctgtc

SEQ ID NO: 463 - DNA, 30 bp, Artificial Sequence
  Primer
catgatcagctgggccaagaacaagtgccc

SEQ ID NO: 464 - DNA, 14 bp, Artificial Sequence
  Primer
actgcagtgagggc

SEQ ID NO: 465 - DNA, 30 bp, Artificial Sequence
  Primer
catgatcagctgggccaagaagccacagtc

SEQ ID NO: 466 - DNA, 18 bp, Artificial Sequence
  Primer
agatcagaaggtgactgt

SEQ ID NO: 467 - DNA, 28 bp, Artificial Sequence
  Primer
catgatcagctgggccaagaacgtggat

SEQ ID NO: 468 - DNA, 19 bp, Artificial Sequence
  Primer
atcgtagaggaaaatccac

SEQ ID NO: 469 - DNA, 17 bp, Artificial Sequence
  Primer
atgacctatgatttgac

SEQ ID NO: 470 - DNA, 29 bp, Artificial Sequence
  Primer
catgatcagctgggccaagatgtgaacaa

SEQ ID NO: 471 - DNA, 17 bp, Artificial Sequence
  Primer
attcctagaaattgttc

SEQ ID NO: 472 - DNA, 29 bp, Artificial Sequence
  Primer
catgatcagctgggccaagatacctgcac

SEQ ID NO: 473 - DNA, 16 bp, Artificial Sequence
  Primer
caaagtgctaacagtg

SEQ ID NO: 474 - DNA, 30 bp, Artificial Sequence
  Primer
catgatcagctgggccaagatccaaaacat

SEQ ID NO: 475 - DNA, 30 bp, Artificial Sequence
  Primer
catgatcagctgggccaagagaacaggtag

SEQ ID NO: 476 - DNA, 14 bp, Artificial Sequence
  Primer
cctagtaggtgctc

SEQ ID NO: 477 - DNA, 15 bp, Artificial Sequence
  Primer
actagactgaggctc

SEQ ID NO: 478 - DNA, 14 bp, Artificial Sequence
  Primer
gcaaagcacagggc

SEQ ID NO: 479 - DNA, 31 bp, Artificial Sequence
  Primer
catgatcagctgggccaagaacggcattacc

SEQ ID NO: 480 - DNA, 18 bp, Artificial Sequence
  Primer
taatactgtctggtaatg

SEQ ID NO: 481 - DNA, 29 bp, Artificial Sequence
  Primer
catgatcagctgggccaagaatccagtca

SEQ ID NO: 482 - DNA, 14 bp, Artificial Sequence
  Primer
taggcagtgtaatt

SEQ ID NO: 483 - DNA, 18 bp, Artificial Sequence
  Primer
tatgtagtatggtccaca

SEQ ID NO: 484 - DNA, 30 bp, Artificial Sequence
  Primer
catgatcagctgggccaagagcactggact

SEQ ID NO: 485 - DNA, 14 bp, Artificial Sequence
  Primer
tgctgacccctagt

SEQ ID NO: 486 - DNA, 29 bp, Artificial Sequence
  Primer
catgatcagctgggccaagaaacaaaatc

SEQ ID NO: 487 - DNA, 18 bp, Artificial Sequence
  Primer
tggaagacttgtgatttt

SEQ ID NO: 488 - DNA, 14 bp, Artificial Sequence
  Primer
tgtctgcccgagtg

SEQ ID NO: 489 - DNA, 20 bp, Artificial Sequence
  Primer
ttaatgctaattgtgatagg

SEQ ID NO: 490 - DNA, 15 bp, Artificial Sequence
  Primer
ttcagctcctatatg

SEQ ID NO: 491 - DNA, 29 bp, Artificial Sequence
  Primer
catgatcagctgggccaagaaggcaaagg

SEQ ID NO: 492 - DNA, 31 bp, Artificial Sequence
  Primer
catgatcagctgggccaagaacacaaattcg

SEQ ID NO: 493 - DNA, 16 bp, Artificial Sequence
  Primer
ccctgtagaaccgaat

SEQ ID NO: 494 - DNA, 15 bp, Artificial Sequence
  Primer
tcacagtgaaccggt

SEQ ID NO: 495 - DNA, 15 bp, Artificial Sequence
  Primer
agctggtgttgtgaa

SEQ ID NO: 496 - DNA, 16 bp, Artificial Sequence
  Primer
tgagatgaagcactgt

SEQ ID NO: 497 - DNA, 16 bp, Artificial Sequence
  Primer
tctcccaacccttgta

SEQ ID NO: 498 - DNA, 15 bp, Artificial Sequence
  Primer
aacattcaacgctgt

SEQ ID NO: 499 - DNA, 16 bp, Artificial Sequence
  Primer
tgtaacagcaactcca